SEQUENCE LISTING (168 sequences)

SEQ ID NO: 1 - DNA, 36 bp, Artificial Sequence
  Synthetic
gcacgtagtataatcaactttgaaaaactgtaataa

SEQ ID NO: 2 - DNA, 36 bp, Artificial Sequence
  Synthetic
gcacgttctattatcaacttcgaaaaactataataa

SEQ ID NO: 3 - DNA, 36 bp, Artificial Sequence
  Synthetic
gcccgcagtattatcaatttcgaaaaattataataa

SEQ ID NO: 4 - DNA, 36 bp, Artificial Sequence
  Synthetic
gcgcgctctataattaacttcgaaaaactttaataa

SEQ ID NO: 5 - DNA, 36 bp, Artificial Sequence
  Synthetic
gcacgctccattattaactttgaaaaactttaataa

SEQ ID NO: 6 - DNA, 36 bp, Artificial Sequence
  Synthetic
gctcgctctatcatcaatttcgaaaaactttaataa

SEQ ID NO: 7 - DNA, 36 bp, Artificial Sequence
  Synthetic
gcacgtagtattattaacttcgaaaagttataataa

SEQ ID NO: 8 - DNA, 36 bp, Artificial Sequence
  Synthetic
gcacgttccatcattaactttgaaaaactataataa

SEQ ID NO: 9 - DNA, 36 bp, Artificial Sequence
  Synthetic
gctcgctcaatcatcaactttgaaaagctataataa

SEQ ID NO: 10 - DNA, 36 bp, Artificial Sequence
  Synthetic
gctcgctctatcatcaacttcgaaaaattgtaataa

SEQ ID NO: 11 - DNA, 36 bp, Artificial Sequence
  Synthetic
gctcgctctattatcaattttgaaaaattataataa

SEQ ID NO: 12 - DNA, 36 bp, Artificial Sequence
  Synthetic
gctcgtagtattattaatttcgaaaaattataataa

SEQ ID NO: 13 - DNA, 36 bp, Artificial Sequence
  Synthetic
gctcgttcgattatcaacttcgaaaaactgtaataa

SEQ ID NO: 14 - DNA, 36 bp, Artificial Sequence
  Synthetic
gcaagaagcatcatcaacttcgaaaaactgtaataa

SEQ ID NO: 15 - DNA, 36 bp, Artificial Sequence
  Synthetic
gcgcgttctattattaattttgaaaaattataataa

SEQ ID NO: 16 - protein, 10 aa, Artificial Sequence
  Synthetic
ARSIINFEKL

SEQ ID NO: 17 - DNA, 66 bp, Artificial Sequence
  Synthetic
gattataaagatcatgacggagactataaagaccatgacattgattacaaagacgacgatgacaaa

SEQ ID NO: 18 - DNA, 66 bp, Artificial Sequence
  Synthetic
gactataaagaccacgatggcgattataaagaccatgatattgactacaaagatgatgatgataag

SEQ ID NO: 19 - DNA, 66 bp, Artificial Sequence
  Synthetic
gattataaagatcatgatggcgactataaagatcatgatatcgattacaaagatgacgatgacaaa

SEQ ID NO: 20 - DNA, 66 bp, Artificial Sequence
  Synthetic
gactacaaagatcacgatggtgactacaaagatcacgacattgattataaagacgatgatgacaaa

SEQ ID NO: 21 - DNA, 66 bp, Artificial Sequence
  Synthetic
gattacaaagatcacgatggtgattataaggatcacgatattgattacaaagacgacgacgataaa

SEQ ID NO: 22 - DNA, 66 bp, Artificial Sequence
  Synthetic
gattacaaagatcacgatggcgattacaaagatcatgacattgactacaaagacgatgatgataaa

SEQ ID NO: 23 - DNA, 66 bp, Artificial Sequence
  Synthetic
gattacaaggatcatgatggtgattacaaagatcacgatatcgactacaaagatgatgacgataaa

SEQ ID NO: 24 - DNA, 66 bp, Artificial Sequence
  Synthetic
gactacaaagatcatgatggtgattacaaagatcatgacattgattataaagatgatgatgacaaa

SEQ ID NO: 25 - DNA, 66 bp, Artificial Sequence
  Synthetic
gattataaagaccatgatggtgattataaggatcatgatatcgattataaggatgacgacgataaa

SEQ ID NO: 26 - DNA, 66 bp, Artificial Sequence
  Synthetic
gattataaagatcacgatggcgattataaagaccacgatattgattataaagacgacgatgacaaa

SEQ ID NO: 27 - DNA, 66 bp, Artificial Sequence
  Synthetic
gactataaagaccacgatggtgattataaagatcacgacatcgactacaaagacgatgatgataaa

SEQ ID NO: 28 - DNA, 66 bp, Artificial Sequence
  Synthetic
gactacaaagatcacgacggcgattataaagatcacgatattgactataaagatgacgatgataaa

SEQ ID NO: 29 - DNA, 66 bp, Artificial Sequence
  Synthetic
gattataaagaccatgatggagattacaaagatcatgatattgactataaagacgacgacgataaa

SEQ ID NO: 30 - DNA, 66 bp, Artificial Sequence
  Synthetic
gattataaagatcacgatggtgactacaaagatcacgatatcgattataaagacgatgacgataaa

SEQ ID NO: 31 - DNA, 66 bp, Artificial Sequence
  Synthetic
gactacaaagatcacgatggtgattataaagaccatgatattgattacaaagatgatgatgacaaa

SEQ ID NO: 32 - protein, 22 aa, Artificial Sequence
  Synthetic
DYKDHDGDYKDHDIDYKDDDDK

SEQ ID NO: 33 - protein, 6 aa, Artificial Sequence
  Synthetic
GASGAS

SEQ ID NO: 34 - protein, 6 aa, Artificial Sequence
  Synthetic
GSAGSA

SEQ ID NO: 35 - protein, 4 aa, Artificial Sequence
  Synthetic
GGGG

SEQ ID NO: 36 - protein, 5 aa, Artificial Sequence
  Synthetic
GGGGS

SEQ ID NO: 37 - protein, 8 aa, Artificial Sequence
  Synthetic
VGKGGSGG

SEQ ID NO: 38 - protein, 5 aa, Artificial Sequence
  Synthetic
PAPAP

SEQ ID NO: 39 - protein, 5 aa, Artificial Sequence
  Synthetic
EAAAK

SEQ ID NO: 40 - protein, 6 aa, Artificial Sequence
  Synthetic
AYLAYL

SEQ ID NO: 41 - protein, 6 aa, Artificial Sequence
  Synthetic
LRALRA

SEQ ID NO: 42 - protein, 4 aa, Artificial Sequence
  Synthetic
RLRA

SEQ ID NO: 43 - protein, 32 aa, Artificial Sequence
  Synthetic
KENSISSMAPPASPPASPKTPIEKKHADEIDK

SEQ ID NO: 44 - protein, 19 aa, Artificial Sequence
  Synthetic
KENSISSMAPPASPPASPK

SEQ ID NO: 45 - protein, 14 aa, Artificial Sequence
  Synthetic
KTEEQPSEVNTGPR

SEQ ID NO: 46 - protein, 28 aa, Artificial Sequence
  Synthetic
KESVVDASESDLDSSMQSADESTPQPLK

SEQ ID NO: 47 - protein, 20 aa, Artificial Sequence
  Synthetic
KSEEVNASDFPPPPTDEELR

SEQ ID NO: 48 - protein, 33 aa, Artificial Sequence
  Synthetic
RGGRPTSEEFSSLNSGDFTDDENSETTEEEIDR

SEQ ID NO: 49 - protein, 17 aa, Artificial Sequence
  Synthetic
KQNTASTETTTTNEQPK

SEQ ID NO: 50 - protein, 17 aa, Artificial Sequence
  Synthetic
KQNTANTETTTTNEQPK

SEQ ID NO: 51 - protein, 19 aa, Artificial Sequence
  Synthetic
RSEVTISPAETPESPPATP

SEQ ID NO: 52 - protein, 28 aa, Artificial Sequence
  Synthetic
KASVTDTSEGDLDSSMQSADESTPQPLK

SEQ ID NO: 53 - protein, 20 aa, Artificial Sequence
  Synthetic
KNEEVNASDFPPPPTDEELR

SEQ ID NO: 54 - protein, 33 aa, Artificial Sequence
  Synthetic
RGGIPTSEEFSSLNSGDFTDDENSETTEEEIDR

SEQ ID NO: 55 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 56 - protein, 529 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 57 - protein, 441 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 58 - protein, 416 aa, Artificial Sequence
  Synthetic
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 59 - protein, 441 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 60 - DNA, 1323 bp, Artificial Sequence
  Synthetic
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgat

SEQ ID NO: 61 - protein, 633 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 62 - protein, 639 aa, Artificial Sequence
  Synthetic
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSEEFSSLNSGDFTDDENSETTEEEIDRLADLRDRGTGKHSRNAGFLPLNPFISSPVPSLTPKVPKISAPALISDITKKAPFKNPSQPLNVFNKKTTTKTVTKKPTPVKTAPKLAELPATKPQETVLRENKTPFIEKQAETNKQSINMPSLPVIQKEATESDKEEMKPQTEEKMVEESESANNANGKNRSAGIEEGKLIAKSAEDEKAKEEPGNHTTLILAMLAIGVFSLGAFIKIIQLRKNN

SEQ ID NO: 63 - protein, 93 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASG

SEQ ID NO: 64 - protein, 200 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 65 - protein, 303 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSS

SEQ ID NO: 66 - protein, 370 aa, Artificial Sequence
  Synthetic
ATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRPTSE

SEQ ID NO: 67 - protein, 390 aa, Artificial Sequence
  Synthetic
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 68 - DNA, 1170 bp, Artificial Sequence
  Synthetic
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 69 - protein, 100 aa, Artificial Sequence
  Synthetic
MGLNRFMRAMMVVFITANCITINPDIIFAATDSEDSSLNTDEWEEEKTEEQPSEVNTGPRYETAREVSSRDIKELEKSNKVRNTNKADLIAMLKEKAEKG

SEQ ID NO: 70 - protein, 390 aa, Artificial Sequence
  Synthetic
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 71 - DNA, 1170 bp, Artificial Sequence
  Synthetic
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 72 - protein, 226 aa, Artificial Sequence
  Synthetic
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 73 - protein, 5 aa, Artificial Sequence
  Synthetic
QDNKR

SEQ ID NO: 74 - protein, 11 aa, Artificial Sequence
  Synthetic
ECTGLAWEWWR

SEQ ID NO: 75 - protein, 11 aa, Artificial Sequence
  Synthetic
ESLLMWITQCR

SEQ ID NO: 76 - protein, 368 aa, Listeria monocytogenes
MVTGWHRPTWIEIDRAAIRENIKNEQNKLPESVDLWAVVKANAYGHGIIEVARTAKEAGAKGFCVAILDEALALREAGFQDDFILVLGATRKEDANLAAKNHISLTVFREDWLENLTLEATLRIHLKVDSGMGRLGIRTTEEARRIEATSTNDHQLQLEGIYTHFATADQLETSYFEQQLAKFQTILTSLKKRPTYVHTANSAASLLQPQIGFDAIRFGISMYGLTPSTEIKTSLPFELKPALALYTEMVHVKELAPGDSVSYGATYTATEREWVATLPIGYADGLIRHYSGFHVLVDGEPAPIIGRVCMDQTIIKLPREFQTGSKVTIIGKDHGNTVTADDAAQYLDTINYEVTCLLNERIPRKYIH

SEQ ID NO: 77 - protein, 289 aa, Listeria monocytogenes
MKVLVNNHLVEREDATVDIEDRGYQFGDGVYEVVRLYNGKFFTYNEHIDRLYASAAKIDLVIPYSKEELRELLEKLVAENNINTGNVYLQVTRGVQNPRNHVIPDDFPLEGVLTAAAREVPRNERQFVEGGTAITEEDVRWLRCDIKSLNLLGNILAKNKAHQQNALEAILHRGEQVTECSASNVSIIKDGVLWTHAADNLILNGITRQVIIDVAKKNGIPVKEADFTLTDLREADEVFISSTTIEITPITHIDGVQVADGKRGPITAQLHQYFVEEITRACGELEFAK

SEQ ID NO: 78 - DNA, 1107 bp, Listeria monocytogenes
atggtgacaggctggcatcgtccaacatggattgaaatagaccgcgcagcaattcgcgaaaatataaaaaatgaacaaaataaactcccggaaagtgtcgacttatgggcagtagtcaaagctaatgcatatggtcacggaattatcgaagttgctaggacggcgaaagaagctggagcaaaaggtttctgcgtagccattttagatgaggcactggctcttagagaagctggatttcaagatgactttattcttgtgcttggtgcaaccagaaaagaagatgctaatctggcagccaaaaaccacatttcacttactgtttttagagaagattggctagagaatctaacgctagaagcaacacttcgaattcatttaaaagtagatagcggtatggggcgtctcggtattcgtacgactgaagaagcacggcgaattgaagcaaccagtactaatgatcaccaattacaactggaaggtatttacacgcattttgcaacagccgaccagctagaaactagttattttgaacaacaattagctaagttccaaacgattttaacgagtttaaaaaaacgaccaacttatgttcatacagccaattcagctgcttcattgttacagccacaaatcgggtttgatgcgattcgctttggtatttcgatgtatggattaactccctccacagaaatcaaaactagcttgccgtttgagcttaaacctgcacttgcactctataccgagatggttcatgtgaaagaacttgcaccaggcgatagcgttagctacggagcaacttatacagcaacagagcgagaatgggttgcgacattaccaattggctatgcggatggattgattcgtcattacagtggtttccatgttttagtagacggtgaaccagctccaatcattggtcgagtttgtatggatcaaaccatcataaaactaccacgtgaatttcaaactggttcaaaagtaacgataattggcaaagatcatggtaacacggtaacagcagatgatgccgctcaatatttagatacaattaattatgaggtaacttgtttgttaaatgagcgcatacctagaaaatacatccattag

SEQ ID NO: 79 - DNA, 870 bp, Listeria monocytogenes
atgaaagtattagtaaataaccatttagttgaaagagaagatgccacagttgacattgaagaccgcggatatcagtttggtgatggtgtatatgaagtagttcgtctatataatggaaaattctttacttataatgaacacattgatcgcttatatgctagtgcagcaaaaattgacttagttattccttattccaaagaagagctacgtgaattacttgaaaaattagttgccgaaaataatatcaatacagggaatgtctatttacaagtgactcgtggtgttcaaaacccacgtaatcatgtaatccctgatgatttccctctagaaggcgttttaacagcagcagctcgtgaagtacctagaaacgagcgtcaattcgttgaaggtggaacggcgattacagaagaagatgtgcgctggttacgctgtgatattaagagcttaaaccttttaggaaatattctagcaaaaaataaagcacatcaacaaaatgctttggaagctattttacatcgcggggaacaagtaacagaatgttctgcttcaaacgtttctattattaaagatggtgtattatggacgcatgcggcagataacttaatcttaaatggtatcactcgtcaagttatcattgatgttgcgaaaaagaatggcattcctgttaaagaagcggatttcactttaacagaccttcgtgaagcggatgaagtgttcatttcaagtacaactattgaaattacacctattacgcatattgacggagttcaagtagctgacggaaaacgtggaccaattacagcgcaacttcatcaatattttgtagaagaaatcactcgtgcatgtggcgaattagagtttgcaaaataa

SEQ ID NO: 80 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 81 - DNA, 714 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgatttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatcttgattatctcaaaagatatgcccctaaattagatgaatggttttatttagcatgtcctgctacttggggaaaattaaattaa

SEQ ID NO: 82 - protein, 237 aa, Artificial Sequence
  Synthetic
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNVFSINGKLGSICGQLLILTYVYGKETPDGIKITLDNLTMQELGYSSGIAHSSAVSRIISKLKQEKVIVYKNSCFYVQNRDYLKRYAPKLDEWFYLACPATWGKLN

SEQ ID NO: 83 - DNA, 713 bp, Artificial Sequence
  Synthetic
atgaacgctcaagcagaagaattcaaaaaatatttagaaactaacgggataaaaccaaaacaatttcataaaaaagaacttatttttaaccaatgggatccacaagaatattgtatttttctatatgatggtatcacaaagctcacgagtattagcgagaacgggaccatcatgaatttacaatactacaaaggggctttcgttataatgtctggctttattgatacagaaacatcggttggctattataatttagaagtcattagcgagcaggctaccgcatacgttatcaaaataaacgaactaaaagaactactgagcaaaaatcttacgcactttttctatgttttccaaaccctacaaaaacaagtttcatacagcctagctaaatttaatgttttttcgattaacgggaagcttggctctatttgcggtcaacttttaatcctgacctatgtgtatggtaaagaaactcctgatggcatcaagattacactggataatttaacaatgcaggagttaggatattcaagtggcatcgcacatagctcagctgttagcagaattatttccaaattaaagcaagagaaagttatcgtgtataaaaattcatgcttttatgtacaaaatctgattatctcaaaagatatgcccctaaattagatgaatggttttatttagcatgtcctgctacttggggaaaattaaattaa

SEQ ID NO: 84 - DNA, 12 bp, Artificial Sequence
  Synthetic
ggtggtggagga

SEQ ID NO: 85 - DNA, 12 bp, Artificial Sequence
  Synthetic
ggtggaggtgga

SEQ ID NO: 86 - DNA, 12 bp, Artificial Sequence
  Synthetic
ggtggaggaggt

SEQ ID NO: 87 - DNA, 12 bp, Artificial Sequence
  Synthetic
ggaggtggtgga

SEQ ID NO: 88 - DNA, 12 bp, Artificial Sequence
  Synthetic
ggaggaggtggt

SEQ ID NO: 89 - DNA, 12 bp, Artificial Sequence
  Synthetic
ggaggtggaggt

SEQ ID NO: 90 - DNA, 12 bp, Artificial Sequence
  Synthetic
ggaggaggaggt

SEQ ID NO: 91 - DNA, 12 bp, Artificial Sequence
  Synthetic
ggaggaggtgga

SEQ ID NO: 92 - DNA, 12 bp, Artificial Sequence
  Synthetic
ggaggtggagga

SEQ ID NO: 93 - DNA, 12 bp, Artificial Sequence
  Synthetic
ggtggaggagga

SEQ ID NO: 94 - DNA, 12 bp, Artificial Sequence
  Synthetic
ggaggaggagga

SEQ ID NO: 95 - protein, 8 aa, Homo sapiens
RMFPNAPY

SEQ ID NO: 96 - protein, 17 aa, Artificial Sequence
  Synthetic
ARSIINFEKLSHHHHHH

SEQ ID NO: 97 - protein, 451 aa, Artificial Sequence
  Synthetic
MGSDVRDLNALLPAVPSLGGGGGCALPVSGAAQWAPVLDFAPPGASAYGSLGGHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLALARSIINFEKLSHHHHHH

SEQ ID NO: 98 - protein, 8 aa, Artificial Sequence
  Synthetic
SIINFEKL

SEQ ID NO: 99 - protein, 21 aa, Artificial Sequence
  Synthetic
DYKDHDGDYKDHDIDYKDDDK

SEQ ID NO: 100 - protein, 75 aa, Artificial Sequence [sequence omitted]

SEQ ID NO: 101 - protein, 9 aa, Homo sapiens
RMFPNAPYL

SEQ ID NO: 102 - protein, 9 aa, Homo sapiens
SLGEQQYSV

SEQ ID NO: 103 - protein, 9 aa, Homo sapiens
ALLPAVPSL

SEQ ID NO: 104 - protein, 9 aa, Homo sapiens
NLGATLKGV

SEQ ID NO: 105 - protein, 9 aa, Homo sapiens
DLNALLPAV

SEQ ID NO: 106 - protein, 9 aa, Homo sapiens
GVFRGIQDV

SEQ ID NO: 107 - protein, 9 aa, Homo sapiens
KRYFKLSHL

SEQ ID NO: 108 - protein, 9 aa, Homo sapiens
ALLLRTPYS

SEQ ID NO: 109 - protein, 9 aa, Homo sapiens
CMTWNQMNL

SEQ ID NO: 110 - protein, 9 aa, Homo sapiens
NMHQRNMTK

SEQ ID NO: 111 - protein, 9 aa, Homo sapiens
QMNLGATLK

SEQ ID NO: 112 - protein, 10 aa, Homo sapiens
FMCAYPGCNK

SEQ ID NO: 113 - protein, 10 aa, Homo sapiens
KLSHLQMHSR

SEQ ID NO: 114 - protein, 9 aa, Artificial Sequence
  Synthetic
FMFPNAPYL

SEQ ID NO: 115 - protein, 9 aa, Artificial Sequence
  Synthetic
YMFPNAPYL

SEQ ID NO: 116 - protein, 9 aa, Artificial Sequence
  Synthetic
YLGEQQYSV

SEQ ID NO: 117 - protein, 9 aa, Artificial Sequence
  Synthetic
YLLPAVPSL

SEQ ID NO: 118 - protein, 9 aa, Artificial Sequence
  Synthetic
YLGATLKGV

SEQ ID NO: 119 - protein, 9 aa, Artificial Sequence
  Synthetic
YLNALLPAV

SEQ ID NO: 120 - protein, 9 aa, Artificial Sequence
  Synthetic
GLRRGIQDV

SEQ ID NO: 121 - protein, 9 aa, Artificial Sequence
  Synthetic
KLYFKLSHL

SEQ ID NO: 122 - protein, 9 aa, Artificial Sequence
  Synthetic
ALLLRTPYV

SEQ ID NO: 123 - protein, 9 aa, Artificial Sequence
  Synthetic
YMTWNQMNL

SEQ ID NO: 124 - protein, 9 aa, Artificial Sequence
  Synthetic
NMYQRNMTK

SEQ ID NO: 125 - protein, 9 aa, Artificial Sequence
  Synthetic
NMHQRVMTK

SEQ ID NO: 126 - protein, 9 aa, Artificial Sequence
  Synthetic
NMYQRVMTK

SEQ ID NO: 127 - protein, 9 aa, Artificial Sequence
  Synthetic
QMYLGATLK

SEQ ID NO: 128 - protein, 9 aa, Artificial Sequence
  Synthetic
QMNLGVTLK

SEQ ID NO: 129 - protein, 9 aa, Artificial Sequence
  Synthetic
QMYLGVTLK

SEQ ID NO: 130 - protein, 10 aa, Artificial Sequence
  Synthetic
FMYAYPGCNK

SEQ ID NO: 131 - protein, 10 aa, Artificial Sequence
  Synthetic
FMCAYPFCNK

SEQ ID NO: 132 - protein, 10 aa, Artificial Sequence
  Synthetic
FMYAYPFCNK

SEQ ID NO: 133 - protein, 10 aa, Artificial Sequence
  Synthetic
KLYHLQMHSR

SEQ ID NO: 134 - protein, 10 aa, Artificial Sequence
  Synthetic
KLSHLQMHSK

SEQ ID NO: 135 - protein, 10 aa, Artificial Sequence
  Synthetic
KLYHLQMHSK

SEQ ID NO: 136 - protein, 19 aa, Artificial Sequence
  Synthetic
SGQAFMFPNAPYLPSCLES

SEQ ID NO: 137 - protein, 19 aa, Artificial Sequence
  Synthetic
SGQAYMFPNAPYLPSCLES

SEQ ID NO: 138 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 139 - protein, 434 aa, Artificial Sequence
  Synthetic
MGSDVRDLNALLPAVPSLGGGGGCALPVSGAAQWAPVLDFAPPGASAYGSLGGHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 140 - protein, 288 aa, Homo sapiens
MEKGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 141 - protein, 302 aa, Homo sapiens
MEKGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 142 - protein, 517 aa, Homo sapiens
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 143 - protein, 514 aa, Homo sapiens
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 144 - protein, 497 aa, Homo sapiens
MQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 145 - protein, 449 aa, Homo sapiens
MGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSEDLVRHHNMHQRNMTKLQLAL

SEQ ID NO: 146 - protein, 453 aa, Homo sapiens
AAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 147 - protein, 514 aa, Homo sapiens; identical to SEQ ID NO: 143

SEQ ID NO: 148 - protein, 168 aa, Homo sapiens
MGHHHHHHHHHHSSGHIEGRHMRRVPGVAPTLVRSASETSEKRPFMCAYPGCNKRYFKLSHLQMHSRKHTGEKPYQCDFKDCERRFFRSDQLKRHQRRHTGVKPFQCKTCQRKFSRSDHLKTHTRTHTGEKPFSCRWPSCQKKFARSDELVRHHNMHQRNMTKLQLAL

SEQ ID NO: 149 - protein, 529 aa, Artificial Sequence
  Synthetic
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKEATGLAWEAARTVIDDRNLPLVKNRNISIWGTTLYPKYSNKVDNPIE

SEQ ID NO: 150 - protein, 25 aa, Artificial Sequence
  Synthetic
MKKIMLVFITLILVSLPIAQQTEAK

SEQ ID NO: 151 - protein, 29 aa, Artificial Sequence
  Synthetic
MGLNRFMRAMMVVFITANCITINPDIIFA

SEQ ID NO: 152 - protein, 19 aa, Homo sapiens
SGQARMFPNAPYLPSCLES

SEQ ID NO: 153 - protein, 616 aa, Artificial Sequence
  Synthetic
  MISC_FEATURE (1)..(441): tLLO
  MISC_FEATURE (442)..(460): WT1-427 long
  MISC_FEATURE (466)..(487): WT1-331 long
  MISC_FEATURE (493)..(511): WT1-122A1 long
  MISC_FEATURE (512)..(532): FLAG Tag
  MISC_FEATURE (533)..(607): Ubiquitin
  MISC_FEATURE (608)..(616): WT1-A1
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDRSDELVRHHNMHQRNMTKLGGGGGPGCNKRYFKLSHLQMHSRKHTGGGGGGSGQAYMFPNAPYLPSCLESDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGYMFPNAPYL

SEQ ID NO: 154 - protein, 19 aa, Homo sapiens
RSDELVRHHNMHQRNMTKL

SEQ ID NO: 155 - protein, 22 aa, Homo sapiens
PGCNKRYFKLSHLQMHSRKHTG

SEQ ID NO: 156 - protein, 546 aa, Artificial Sequence
  Synthetic
  MISC_FEATURE (1)..(441): tLLO
  MISC_FEATURE (442)..(462): FLAG Tag
  MISC_FEATURE (463)..(537): Ubiquitin
  MISC_FEATURE (538)..(546): WT1-F
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGFMFPNAPYL

SEQ ID NO: 157 - protein, 546 aa, Artificial Sequence
  Synthetic
  MISC_FEATURE (1)..(441): tLLO
  MISC_FEATURE (442)..(462): FLAG Tag
  MISC_FEATURE (463)..(537): Ubiquitin
  MISC_FEATURE (538)..(546): WT1-A1
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGYMFPNAPYL

SEQ ID NO: 158 - protein, 9 aa, Homo sapiens
NQMNLGATL

SEQ ID NO: 159 - protein, 9 aa, Artificial Sequence
  Synthetic
NLMNLGATL

SEQ ID NO: 160 - protein, 9 aa, Artificial Sequence
  Synthetic
NYMNLGATL

SEQ ID NO: 161 - protein, 15 aa, Homo sapiens
WNQMNLGATLKGVAA

SEQ ID NO: 162 - protein, 15 aa, Artificial Sequence
  Synthetic
WNLMNLGATLKGVAA

SEQ ID NO: 163 - protein, 15 aa, Artificial Sequence
  Synthetic
WNYMNLGATLKGVAA

SEQ ID NO: 164 - protein, 546 aa, Artificial Sequence
  Synthetic
  MISC_FEATURE (1)..(441): tLLO
  MISC_FEATURE (442)..(462): FLAG Tag
  MISC_FEATURE (463)..(537): Ubiquitin
  MISC_FEATURE (538)..(546): Wild-Type A24
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGNQMNLGATL

SEQ ID NO: 165 - protein, 546 aa, Artificial Sequence
  Synthetic
  MISC_FEATURE (1)..(441): tLLO
  MISC_FEATURE (442)..(462): FLAG Tag
  MISC_FEATURE (463)..(537): Ubiquitin
  MISC_FEATURE (538)..(546): Heteroclitic A24 v1
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGNLMNLGATL

SEQ ID NO: 166 - protein, 546 aa, Artificial Sequence
  Synthetic
  MISC_FEATURE (1)..(441): tLLO
  MISC_FEATURE (442)..(462): FLAG Tag
  MISC_FEATURE (463)..(537): Ubiquitin
  MISC_FEATURE (538)..(546): Heteroclitic A24 v2
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDDYKDHDGDYKDHDIDYKDDDKQIFVKTLTGKTITLEVEPSDTIENVKAKIQDKEGIPPDQQRLIFAGKQLEDGRTLSDYNIQKESTLHLVLRLRGGNYMNLGATL

SEQ ID NO: 167 - DNA, 16 bp, Artificial Sequence
  Synthetic
catcgatcactctgga

SEQ ID NO: 168 - DNA, 19 bp, Artificial Sequence
  Synthetic
ctaactccaatgttacttg

(19) United States
(12) Patent Application Publication
(10) Pub. No.: US 2019/0248856 A1
(43) Pub. Date: Aug. 15, 2019

(54) Listeria-Based Immunogenic Compositions Comprising Wilms Tumor Protein Antigens And Methods Of Use Thereof

(71) Applicant: Advaxis, Inc., Princeton, NJ (US)

(72) Inventors: Michael Princiotta, Hightstown, NJ (US); Robert Petit, Newtown, PA (US)

(73) Assignee: Advaxis, Inc., Princeton, NJ (US)

(21) Appl. No.: 16/315,082

(22) PCT Filed: Jun. 30, 2017
(86) PCT No.: PCT/US2017/040459

§ 371 Date: Jan. 3, 2019

Related U.S. Application Data
(60) Provisional application No. 62/358,539, filed on Jul. 5, 2016.

(51) Int. Cl. C07K 14/47; C07K 14/195; A61K 39/39; A61K 39/00; A61P 35/00

(52) U.S. Cl. CPC C07K 14/4748; C07K 14/4702; C07K 14/195; C07K 2319/02; A61K 39/001153; A61P 35/00; A61K 39/39

(57) ABSTRACT

Provided are Listeria-based immunogenic compositions comprising Wilms tumor protein (WT1) antigens and methods for treating and vaccinating against cancer and inducing an immune response against the same in a subject. Also provided herein are recombinant fusion polypeptides or chimeric polypeptides comprising Wilms tumor protein antigens, nucleic acids encoding such chimeric polypeptides or fusion polypeptides, recombinant bacteria or Listeria strains comprising such chimeric polypeptides or fusion polypeptides or such nucleic acids, and cell banks comprising such recombinant bacteria or Listeria strains. Also provided herein are methods of generating such chimeric polypeptides or fusion polypeptides, such nucleic acids, and such recombinant bacteria or Listeria strains. Also provided are immunogenic compositions, pharmaceutical compositions, and vaccines comprising such chimeric polypeptides or fusion polypeptides, such nucleic acids, or such recombinant bacteria or Listeria strains. Also provided are methods of inducing an anti-WT1 immune response in a subject, methods of inducing an anti-WT1-expressing-tumor or anti-WT1-expressing-cancer immune response in a subject, methods of treating a WT1-expressing or WT1-associated tumor or cancer in a subject, methods of preventing a WT1-expressing or WT1-associated tumor or cancer in a subject, and methods of protecting a subject against a WT1-expressing or WT1-associated tumor or cancer using such recombinant chimeric polypeptides or fusion polypeptides, nucleic acids, recombinant bacteria or Listeria strains, immunogenic compositions, pharmaceutical compositions, or vaccines.

### CROSS-REFERENCE TO RELATED APPLICATIONS

This application claims the benefit of U.S. Application No. 62/358,539, filed Jul. 5, 2016, herein incorporated by reference in its entirety for all purposes.

### REFERENCE TO A SEQUENCE LISTING SUBMITTED AS A TEXT FILE VIA EFS WEB

The Sequence Listing written in file 499335SEQLIST.txt is 171 kilobytes, was created on Jun. 30, 2017, and is hereby incorporated by reference.

### BACKGROUND

The Wilms tumor protein (WT1) is overexpressed in a number of cancers and may play an oncologic role in hematologic malignancies and a variety of solid tumors, including leukemia, breast cancer, ovarian cancer, glioblastoma, soft tissue sarcoma, and other cancers. WT1 may be a promising target for immunotherapy and for harnessing the immune system to treat patients with cancers associated with expression of WT1.

Listeria monocytogenes (Lm) is a gram positive, facultative intracellular bacterium that has direct access to the cytoplasm of antigen presenting cells, such as macrophages and dendritic cells, largely due to the pore-forming activity of listeriolysin-O (LLO). LLO is secreted by Lm following engulfment by the cells and perforates the phagolysosomal membrane, allowing the bacterium to escape the vacuole and enter the cytoplasm. LLO is very efficiently presented to the immune system via MHC class I molecules. Furthermore, Lm-derived peptides also have access to MHC class II presentation via the phagolysosome.

### SUMMARY

Methods and compositions are provided for cancer immunotherapy for treatment or prophylaxis of Wilms tumor protein (WT1)-expressing or WT1-associate tumors. In one aspect, provided herein are minigene constructs comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide. The antigenic WT1 peptides can be native WT1 peptides and/or heteroclitic mutant WT1 peptides. Also provided are chimeric polypeptides encoded by such minigene constructs and recombinant Listeria strains comprising such minigene constructs.

In another aspect, provided are recombinant Listeria strains comprising a nucleic acid comprising a first open reading frame encoding a fusion polypeptide, wherein the fusion polypeptide comprises a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136. Also provided are such fusion polypeptides and nucleic acids encoding such fusion polypeptides.

In another aspect, provided herein are immunogenic compositions, pharmaceutical compositions, or vaccines comprising a recombinant Listeria strain comprising a minigene construct comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide or comprising a nucleic acid comprising a first open reading frame encoding a fusion polypeptide, wherein the fusion polypeptide comprises a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136. Also provided are immunogenic compositions, pharmaceutical compositions, or vaccines comprising the chimeric polypeptide or fusion polypeptide or a nucleic acid encoding the chimeric polypeptide or fusion polypeptide.

In another aspect, provided herein are methods of inducing an immune response against a WT1-expressing or WT1-associated tumor or cancer in a subject, comprising administering to the subject a recombinant Listeria strain comprising a minigene construct comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide or comprising a nucleic acid comprising a first open reading frame encoding a fusion polypeptide, wherein the fusion polypeptide comprises a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136. Also provided are methods of inducing an immune response against a WT1-expressing or WT1-associated tumor or cancer in a subject, comprising administering to the subject an immunogenic composition, a pharmaceutical composition, or a vaccine comprising such a recombinant Listeria strain. Also provided are methods of inducing an immune response against a WT1-expressing or WT1-associated tumor or cancer in a subject, comprising administering to the subject the chimeric polypeptide or fusion polypeptide or a nucleic acid encoding the chimeric polypeptide (e.g., the minigene construct) or fusion polypeptide, an immunogenic composition comprising the chimeric polypeptide or fusion polypeptide or the nucleic acid encoding the chimeric polypeptide (e.g., the minigene construct) or fusion polypeptide, a pharmaceutical composition comprising the chimeric polypeptide or fusion polypeptide or the nucleic acid encoding the chimeric polypeptide (e.g., the minigene construct) or fusion polypeptide, or a vaccine comprising the chimeric polypeptide or fusion polypeptide or the nucleic acid encoding the chimeric polypeptide (e.g., the minigene construct) or fusion polypeptide.

In another aspect, provided herein are methods of preventing or treating a WT1-expressing or WT1-associated tumor or cancer in a subject, comprising administering to the subject a recombinant Listeria strain comprising a minigene construct comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide or comprising a nucleic acid comprising a first open reading frame encoding a fusion polypeptide, wherein the fusion polypeptide comprises a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136. Also provided are methods of preventing or treating a WT1-expressing or WT1-associated tumor or cancer in a subject, comprising administering to the subject an immunogenic composition, a pharmaceutical composition, or a vaccine comprising such a recombinant Listeria strain. Also provided are methods of preventing or treating a WT1-expressing or WT1-associated tumor or cancer in a subject, comprising administering to the subject the chimeric polypeptide or fusion polypeptide, a nucleic acid encoding the chimeric polypeptide (e.g., the minigene construct) or fusion polypeptide, an immunogenic composition comprising the chimeric polypeptide or fusion polypeptide or the nucleic acid encoding the chimeric polypeptide (e.g., the minigene construct) or fusion polypeptide, a pharmaceutical composition comprising the chimeric polypeptide or fusion polypeptide or the nucleic acid encoding the chimeric polypeptide (e.g., the minigene construct) or fusion polypeptide, or a vaccine comprising the chimeric polypeptide or fusion polypeptide or the nucleic acid encoding the chimeric polypeptide (e.g., the minigene construct) or fusion polypeptide.

In another aspect, provided herein are cell banks comprising one or more recombinant Listeria strains comprising a minigene construct comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide or comprising a nucleic acid comprising a first open reading frame encoding a fusion polypeptide, wherein the fusion polypeptide comprises a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136.

In another aspect, provided is an immunogenic composition comprising a recombinant Listeria strain comprising a recombinant attenuated Listeria strain comprising a nucleic acid encoding a recombinant polypeptide, wherein the recombinant polypeptide comprises a truncated listeriolysin O (LLO) protein, a truncated ActA protein, or a PEST amino acid sequence fused to a Wilms tumor protein or an immunogenic fragment thereof.

In another aspect, provided is a method of treating a cancer in a subject, comprising administering such an immunogenic composition to the subject.

In another aspect, provided is a method of eliciting an enhanced anti-cancer T cell response in a subject, comprising administering such an immunogenic composition to the subject.

In another aspect, provided is a method of eliciting an enhanced immune response against a cancer in a subject, comprising administering such an immunogenic composition to the subject.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIGS. 1A-1C show IFN-γ response to WT1 and Ova peptides in C57BL/6 mice immunized with LmddA323 (WT1) and LmddA324 (Ova). FIG. 1A shows primary response to WT1 and Ova peptide stimulation in mice immunized with WT1 (mouse #'s 1&2) or Ova (mouse #'s 3&4) minigene constructs. FIGS. 1B-1C show secondary response to WT1 and Ova peptide stimulation in mice immunized with WT1 (FIG. 1B) or Ova minigene expressing Lm (FIG. 1C).

FIGS. 2A-2C show expression and secretion levels for a positive control (FIG. 2A), a negative control (FIG. 2B), and WT1 (FIG. 2C) in murine dendritic DC2.4 cells.

FIGS. 3A and 3B show schematics of WT1 minigene constructs. FIG. 3A shows a WT1 minigene construct designed to express a single WT1 chimeric polypeptide antigen. FIG. 3B shows a WT1 minigene construct designed to express three separate WT1 chimeric polypeptide antigens.

FIG. 4 shows an ELISPOT assay in splenocytes stimulated ex vivo with WT1 peptides RMFPNAPYL (SEQ ID NO: 101) and FMFPNAPYL (SEQ ID NO: 114). The splenocytes are from HLA2 transgenic mice immunized with the WT1-F minigene construct. PBS and LmddA274 were used as negative controls.

FIG. 5 shows an ELISPOT assay in splenocytes stimulated ex vivo with WT1 peptides RMFPNAPYL (SEQ ID NO: 101) and YMFPNAPYL (SEQ ID NO: 115). The splenocytes are from HLA2 transgenic mice immunized with the WT1-AH1-Tyr minigene construct. PBS and LmddA274 were used as negative controls.

FIGS. 6A and 6B show IFN-γ spot-forming cells (SFC) per million splenocytes stimulated ex vivo with WT1 peptides RMFPNAPYL (SEQ ID NO: 101; FIG. 6A) and FMFPNAPYL (SEQ ID NO: 114; FIG. 6B). The splenocytes are from HLA2 transgenic mice immunized with the WT1-F minigene construct. PBS and LmddA274 were used as negative controls.

FIGS. 7A and 7B show IFN-γ spot-forming cells (SFC) per million splenocytes stimulated ex vivo with WT1 peptides RMFPNAPYL (SEQ ID NO: 101; FIG. 7A) and YMFPNAPYL (SEQ ID NO: 115; FIG. 7B). The splenocytes are from HLA2 transgenic mice immunized with the WT1-AH1-Tyr minigene construct. PBS and LmddA274 were used as negative controls.

FIGS. 8A and 8B show Western blots of the Lmdda-WT1-tLLO-FLAG-Ub-heteroclitic phenylalanine minigene construct (FIG. 8A) and the Lmdda-WT1-tLLO-P1-P2-P3-FLAG-Ub-heteroclitic tyrosine minigene construct (FIG. 8B). In FIG. 8A, lane 1 is the ladder, lane 2 is the Lmdda-WT1-tLLO-P1-P2-P3-FLAG-Ub-heteroclitic tyrosine minigene construct (68 kDa), and lane 3 is a negative control. In FIG. 8B, lane 1 is the ladder, lane 2 is the negative control, and lane 3 is the WT1-tLLO-FLAG-Ub-heteroclitic phenylalanine minigene construct (construct #1).

FIG. 9 shows colony PCR results for several Lm-minigene constructs expressing heteroclitic mutant WT1 peptides. Mutated residues are bolded and underlined.

### DEFINITIONS

The terms “protein,” “polypeptide,” and “peptide,” used interchangeably herein, refer to polymeric forms of amino acids of any length, including coded and non-coded amino acids and chemically or biochemically modified or derivatized amino acids. The terms include polymers that have been modified, such as polypeptides having modified peptide backbones.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The term “fusion protein” refers to a protein comprising two or more peptides linked together by peptide bonds or other chemical bonds. The peptides can be linked together directly by a peptide or other chemical bond. For example, a chimeric molecule can be recombinantly expressed as a single-chain fusion protein. Alternatively, the peptides can be linked together by a “linker” such as one or more amino acids or another suitable linker between the two or more peptides.

The terms “nucleic acid” and “polynucleotide,” used interchangeably herein, refer to polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

“Codon optimization” refers to a process of modifying a nucleic acid sequence for enhanced expression in particular host cells by replacing at least one codon of the native sequence with a codon that is more frequently or most frequently used in the genes of the host cell while maintaining the native amino acid sequence. For example, a polynucleotide encoding a fusion polypeptide can be modified to substitute codons having a higher frequency of usage in a given Listeria cell or any other host cell as compared to the naturally occurring nucleic acid sequence. Codon usage tables are readily available, for example, at the “Codon Usage Database.” The optimal codons utilized by L. monocytogenes for each amino acid are shown US 2007/0207170, herein incorporated by reference in its entirety for all purposes. These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “plasmid” or “vector” includes any known delivery vector including a bacterial delivery vector, a viral vector delivery vector, a peptide immunotherapy delivery vector, a DNA immunotherapy delivery vector, an episomal plasmid, an integrative plasmid, or a phage vector. The term “vector” refers to a construct which is capable of delivering, and, optionally, expressing, one or more fusion polypeptides in a host cell.

The term “episomal plasmid” or “extrachromosomal plasmid” refers to a nucleic acid vector that is physically separate from chromosomal DNA (i.e., episomal or extrachromosomal and does not integrated into a host cell's genome) and replicates independently of chromosomal DNA. A plasmid may be linear or circular, and it may be single-stranded or double-stranded. Episomal plasmids may optionally persist in multiple copies in a host cell's cytoplasm (e.g., Listeria), resulting in amplification of any genes of interest within the episomal plasmid.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “stably maintained” refers to maintenance of a nucleic acid molecule or plasmid in the absence of selection (e.g., antibiotic selection) for at least 10 generations without detectable loss. For example, the period can be at least 15 generations, 20 generations, at least 25 generations, at least 30 generations, at least 40 generations, at least 50 generations, at least 60 generations, at least 80 generations, at least 100 generations, at least 150 generations, at least 200 generations, at least 300 generations, or at least 500 generations. Stably maintained can refer to a nucleic acid molecule or plasmid being maintained stably in cells in vitro (e.g., in culture), being maintained stably in vivo, or both.

An “open reading frame” or “ORF” is a portion of a DNA which contains a sequence of bases that could potentially encode a protein. As an example, an ORF can be located between the start-code sequence (initiation codon) and the stop-codon sequence (termination codon) of a gene.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety.

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” refers to the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) refers to a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence.

The term “wild type” refers to entities having a structure and/or activity as found in a normal (as contrasted with mutant, diseased, altered, or so forth) state or context. Wild type gene and polypeptides often exist in multiple different forms (e.g., alleles).

The term “isolated” with respect to proteins and nucleic acid refers to proteins and nucleic acids that are relatively purified with respect to other bacterial, viral or cellular components that may normally be present in situ, up to and including a substantially pure preparation of the protein and the polynucleotide. The term “isolated” also includes proteins and nucleic acids that have no naturally occurring counterpart, have been chemically synthesized and are thus substantially uncontaminated by other proteins or nucleic acids, or has been separated or purified from most other cellular components with which they are naturally accompanied (e.g., other cellular proteins, polynucleotides, or cellular components).

“Exogenous” or “heterologous” molecules or sequences are molecules or sequences that are not normally expressed in a cell or are not normally present in a cell in that form. Normal presence includes presence with respect to the particular developmental stage and environmental conditions of the cell. An exogenous or heterologous molecule or sequence, for example, can include a mutated version of a corresponding endogenous sequence within the cell or can include a sequence corresponding to an endogenous sequence within the cell but in a different form (i.e., not within a chromosome). An exogenous or heterologous molecule or sequence in a particular cell can also be a molecule or sequence derived from a different species than a reference species of the cell or from a different organism within the same species. For example, in the case of a Listeria strain expressing a heterologous polypeptide, the heterologous polypeptide could be a polypeptide that is not native or endogenous to the Listeria strain, that is not normally expressed by the Listeria strain, from a source other than the Listeria strain, derived from a different organism within the same species.

In contrast, “endogenous” molecules or sequences or “native” molecules or sequences are molecules or sequences that are normally present in that form in a particular cell at a particular developmental stage under particular environmental conditions.

The term “variant” refers to an amino acid or nucleic acid sequence (or an organism or tissue) that is different from the majority of the population but is still sufficiently similar to the common mode to be considered to be one of them (e.g., splice variants).

The term “isoform” refers to a version of a molecule (e.g., a protein) with only slight differences compared to another isoform, or version (e.g., of the same protein). For example, protein isoforms may be produced from different but related genes, they may arise from the same gene by alternative splicing, or they may arise from single nucleotide polymorphisms.

The term “fragment” when referring to a protein means a protein that is shorter or has fewer amino acids than the full length protein. The term “fragment” when referring to a nucleic acid means a nucleic acid that is shorter or has fewer nucleotides than the full length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment. A fragment can also be, for example, a functional fragment or an immunogenic fragment.

The term “analog” when referring to a protein means a protein that differs from a naturally occurring protein by conservative amino acid differences, by modifications which do not affect amino acid sequence, or by both.

The term “functional” refers to the innate ability of a protein or nucleic acid (or a fragment, isoform, or variant thereof) to exhibit a biological activity or function. Such biological activities or functions can include, for example, the ability to elicit an immune response when administered to a subject. Such biological activities or functions can also include, for example, binding to an interaction partner. In the case of functional fragments, isoforms, or variants, these biological functions may in fact be changed (e.g., with respect to their specificity or selectivity), but with retention of the basic biological function.

The terms “immunogenicity” or “immunogenic” refer to the innate ability of a molecule (e.g., a protein, a nucleic acid, an antigen, or an organism) to elicit an immune response in a subject when administered to the subject. Immunogenicity can be measured, for example, by a greater number of antibodies to the molecule, a greater diversity of antibodies to the molecule, a greater number of T-cells specific for the molecule, a greater cytotoxic or helper T-cell response to the molecule, and the like.

The term “antigen” is used herein to refer to a substance that, when placed in contact with a subject or organism (e.g., when present in or when detected by the subject or organism), results in a detectable immune response from the subject or organism. An antigen may be, for example, a lipid, a protein, a carbohydrate, a nucleic acid, or combinations and variations thereof. For example, an “antigenic peptide” refers to a peptide that leads to the mounting of an immune response in a subject or organism when present in or detected by the subject or organism. For example, such an “antigenic peptide” may encompass proteins that are loaded onto and presented on MHC class I and/or class II molecules on a host cell's surface and can be recognized or detected by an immune cell of the host, thereby leading to the mounting of an immune response against the protein. Such an immune response may also extend to other cells within the host, such as diseased cells (e.g., tumor or cancer cells) that express the same protein.

The term “epitope” refers to a site on an antigen that is recognized by the immune system (e.g., to which an antibody binds). An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

The term “mutation” refers to the any change of the structure of a gene or a protein. For example, a mutation can result from a deletion, an insertion, a substitution, or a rearrangement of chromosome or a protein. An “insertion” changes the number of nucleotides in a gene or the number of amino acids in a protein by adding one or more additional nucleotides or amino acids. A “deletion” changes the number of nucleotides in a gene or the number of amino acids in a protein by reducing one or more additional nucleotides or amino acids.

A “frameshift” mutation in DNA occurs when the addition or loss of nucleotides changes a gene's reading frame. A reading frame consists of groups of 3 bases that each code for one amino acid. A frameshift mutation shifts the grouping of these bases and changes the code for amino acids. The resulting protein is usually nonfunctional. Insertions and deletions can each be frameshift mutations.

A “missense” mutation or substitution refers to a change in one amino acid of a protein or a point mutation in a single nucleotide resulting in a change in an encoded amino acid. A point mutation in a single nucleotide that results in a change in one amino acid is a “nonsynonymous” substitution in the DNA sequence. Nonsynonymous substitutions can also result in a “nonsense” mutation in which a codon is changed to a premature stop codon that results in truncation of the resulting protein. In contrast, a “synonymous” mutation in a DNA is one that does not alter the amino acid sequence of a protein (due to codon degeneracy).

The term “somatic mutation” includes genetic alterations acquired by a cell other than a germ cell (e.g., sperm or egg). Such mutations can be passed on to progeny of the mutated cell in the course of cell division but are not inheritable. In contrast, a germinal mutation occurs in the germ line and can be passed on to the next generation of offspring.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Compositions or methods “comprising” or “including” one or more recited elements may include other elements not specifically recited. For example, a composition that “comprises” or “includes” a protein may contain the protein alone or in combination with other ingredients.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations±0.5%, 1%, 5%, or 10% from a specified value.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### DETAILED DESCRIPTION

### I. Overview

Provided are immunogenic compositions comprising Wilms tumor protein (WT1) antigens and methods for treating and vaccinating against WT1-expressing or WT1-associated tumors or cancers and inducing an immune response against the same in a subject. As one example, provided herein are minigene constructs comprising a nucleic acid comprising an open reading frame encoding a chimeric polypeptide, wherein the chimeric polypeptide comprises: (a) a bacterial secretion signal sequence; (b) a ubiquitin protein; and (c) one or more antigenic WT1 peptides, wherein the bacterial secretion signal sequence, the ubiquitin, and the one or more antigenic WT1 peptides are arranged in tandem from the amino-terminal end to the carboxy-terminal end of the chimeric polypeptide. Also provided herein are the encoded chimeric polypeptides and recombinant bacteria or Listeria strains comprising such minigene constructs or chimeric polypeptides. As another example, provided herein are recombinant fusion polypeptides comprising a PEST-containing peptide fused to an antigenic WT1 peptide selected from a native Wilms tumor protein (WT1), an immunogenic fragment of the native WT1, and a WT1 peptide comprising the sequence set forth in SEQ ID NO: 114 or SEQ ID NO: 136. Also provided herein are nucleic acids encoding such fusion polypeptides and recombinant bacteria or Listeria strains comprising such fusion polypeptides or such nucleic acids.

Also provided are cell banks comprising such recombinant bacteria or Listeria strains; immunogenic compositions, pharmaceutical compositions, and vaccines comprising such chimeric polypeptides or fusion polypeptides, such nucleic acids, or such recombinant bacteria or Listeria strains; and methods of generating such chimeric polypeptides or fusion polypeptides, such nucleic acids, and such recombinant bacteria or Listeria strains. Also provided are methods of inducing an anti-WT1 immune response in a subject, methods of inducing an anti-WT1-expressing-tumor or anti-WT1-expressing-cancer immune response in a subject, methods of treating a WT1-expressing or WT1-associated tumor or cancer in a subject, methods of preventing a WT1-expressing or WT1-associated tumor or cancer in a subject, and methods of protecting a subject against a WT1-expressing or WT1-associated tumor or cancer using such recombinant chimeric polypeptides or fusion polypeptides, nucleic acids, recombinant bacteria or Listeria strains, immunogenic compositions, pharmaceutical compositions, or vaccines.

In a specific example, disclosed herein are compositions comprising a live attenuated recombinant Listeria strain comprising a fusion protein of a truncated listeriolysin O (LLO) protein, a truncated ActA protein, or a PEST amino acid sequence fused to a Wilms tumor protein or an immunogenic fragment thereof. Also disclosed are methods of eliciting an enhanced anti-WT1-expressing-tumor T cell response in a subject with cancer, comprising administering to the subject an affective amount of such a composition. Also disclosed herein are methods of using such compositions for eliciting an enhanced immune response against a cancer in a subject, comprising administering such a composition to the subject having the cancer. Also disclosed herein are methods of using such compositions for treating a cancer in a subject, comprising administering such compositions to the subject. Also disclosed are methods of using such compositions for eliciting an enhanced anti-cancer T cell response in a subject, comprising administering such a composition to the subject having the cancer.

Examples of types of cancer in the methods disclosed herein include any type of cancer known to be associated with expression of Wilms tumor protein. Exemplary cancers include breast cancers such as triple negative breast cancer (TNBC) and gastrointestinal cancers such as esophageal cancer, stomach cancer, gastric cancer, pancreatic cancer, liver cancer, gall bladder cancer, colorectal cancer, anal cancer, and gastrointestinal carcinoid tumors. Triple negative breast cancer (TNBC) refers to any breast cancer that does not express the genes for estrogen receptor (ER), progesterone receptor (PR), or Her2/neu. This makes it more difficult to treat because most chemotherapies target one of the three receptors.

Wilms tumor protein (WT1, AEWS-GUD, NPHS4, WAGR, WIT-2, WT33, Wilms tumor 1) is a protein that in humans is encoded by the WT1 gene on chromosome 11p. This gene encodes a transcription factor that contains four zinc finger motifs at the C-terminus and a proline/glutamine-rich DNA-binding domain at the N-terminus. The WT1 antigen is a transcription factor that is not generally expressed in normal adult cells, but appears in a large number of cancers, as well as in certain cancer stem cells. As an example, full-length Wilms tumor proteins can be used in the methods and compositions disclosed herein, and immunogenic fragments of Wilms tumor proteins such as the fragment set forth in SEQ ID NO: 95 (RMFPNAPY) can be used in the methods and compositions disclosed herein.

Preferably, the recombinant Listeria strain is a Listeria monocytogenes strain. An example of such an attenuated strain is Lm dal(−)dat(−) (Lmdd). Another example of such an attenuated strain is Lm dal(−)dat(−)ΔactA (LmddA). See, e.g., US 2011/0142791, herein incorporated by references in its entirety for all purposes. LmddA is based on a Listeria vaccine vector which is attenuated due to the deletion of the endogenous virulence gene actA. Such strains can retain a plasmid for antigen expression in vivo and in vitro by complementation of the dal gene. Alternatively, the LmddA can be a dal/dat/actA Listeria having mutations in the endogenous dal, dat, and actA genes. Such mutations can be, for example, a deletion, a truncation, or an inactivation of the mutated genes.

Preferably, the Wilms tumor protein or immunogenic fragment thereof is fused to listeriolysin O (LLO) or a fragment thereof. Preferably, the LLO is a truncated LLO (tLLO). Preferably, the tLLO comprises a PEST-like sequence. PEST sequences in eukaryotic proteins have long been identified. For example, proteins containing amino acid sequences that are rich in prolines (P), glutamic acids (E), serines (S) and threonines (T) (PEST), generally, but not always, flanked by clusters containing several positively charged amino acids, have rapid intracellular half-lives (Rogers et al. (1986) Science 234:364-369, herein incorporated by reference in its entirety for all purposes). Further, it has been reported that these sequences target the protein to the ubiquitin-proteosome pathway for degradation (Rechsteiner and Rogers (1996) Trends Biochem. Sci. 21:267-271, herein incorporated by reference in its entirety for all purposes). This pathway is also used by eukaryotic cells to generate immunogenic peptides that bind to MHC class I and it has been hypothesized that PEST sequences are abundant among eukaryotic proteins that give rise to immunogenic peptides (Realini et al. (1994) FEBS Lett. 348:109-113, herein incorporated by reference in its entirety for all purposes). Prokaryotic proteins do not normally contain PEST sequences because they do not have this enzymatic pathway. However, a PEST-like sequence rich in the amino acids proline (P), glutamic acid (E), serine (S) and threonine (T) has been reported at the amino terminus of LLO and has been reported to be essential for L. monocytogenes pathogenicity (Decatur and Portnoy (2000) Science 290:992-995, herein incorporated by reference in its entirety for all purposes). The presence of this PEST-like sequence in LLO targets the protein for destruction by proteolytic machinery of the host cell so that once the LLO has served its function and facilitated the escape of L. monocytogenes from the phagolysosomal vacuole, it is destroyed before it can damage the cells.

Preferably the tLLO refers to a fragment of LLO that is non-hemolytic. For example, the LLO fragment can be rendered non-hemolytic by deletion or mutation of the activation domain. Alternatively, the LLO fragment can be rendered non-hemolytic by deletion or mutation of region comprising cysteine 484. Alternatively, the LLO fragment can be rendered non-hemolytic by a deletion or mutation of the cholesterol binding domain (CBD) as detailed in U.S. Pat. No. 8,771,702, herein incorporated by reference in its entirety for all purposes.

An exemplary N-terminal fragment of an LLO protein utilized in compositions and methods of the present invention has the sequence set forth in (SEQ ID NO: 57). Another exemplary N-terminal fragment of an LLO protein utilized in compositions and methods of the present invention has the sequence set forth in SEQ ID NO: 59.

The Lm technology has a mechanism of action that incorporates potent innate immune stimulation, delivery of a target peptide directly into the cytosol of dendritic cells and antigen presenting cells, generation of a targeted T cell response, and reduced immune suppression by regulatory T cells and myeloid-derived suppressor cells in the tumor microenvironment. Multiple treatments can be given and/or combined without neutralizing antibodies. The Lm technology can use, for example, live, attenuated, bioengineered Lm bacteria to stimulate the immune system to view tumor cells as potentially bacterial-infected cells and target them for elimination. The technology process can start with a live, attenuated strain of Listeria and can add, for example, multiple copies of a plasmid that encodes a fusion protein sequence including a fragment of, for example, the LLO (listeriolysin O) molecule joined to the antigen of interest. This fusion protein is secreted by the Listeria inside antigen-presenting cells. This results in a stimulation of both the innate and adaptive arms of the immune system that reduces tumor defense mechanisms and makes it easier for the immune system to attack and destroy the cancer cells.

WT1 peptides alone are not sufficiently immunogenic upon vaccination and are rapidly degraded. For example, they must be formulated in a squalene oil adjuvant known as Montanide to enhance immunogenicity. Furthermore, the area that will be injected must be pre-treated with injections of the drug filgrastim to induce local inflammation and attract antigen-presenting cells to the area. This combination treatment causes areas of extended induration and multiple injections are required to reach the optimal levels of T cell response, sometimes more than 12 injections. Alternative areas of the body must be injected every time because of the persistence of injection site reactions and delayed-type hypersensitivity. Injected patients develop delayed-type hypersensitivity, which can complicate treatments and may cause early termination. Furthermore, the adjuvant (Montanide) tends to generate more CD4+ T cell responses that it does CD8+ T cell responses because the oil containing the peptides is picked up and presented more through the MHC class 2 system than HMC class 1 system. The Lm-based vectors disclosed herein offer significant immunologic, clinical, practical, and manufacturing improvements over a peptide emulsion in Montanide combined with filgrastim injections.

Immunologically, Lm-based vectors are a far superior platform for the generation of CD8+ dominant T cell responses. First, there is no need to add adjuvants of filgrastim injections. This is because the live attenuated bacteria vectors inherently trigger numerous innate immune activation triggers which include several TLRs, PAMP, and DAMP receptors and have a potent ability to agonize the STING receptor within the cytosol of the antigen-presenting cells. This is a much broader alteration of the immunologic microenvironment that primes the patients' immune system for an adaptive immune response. Second, the Lm vector is infused intravenously. This allows it to reach significantly more antigen-presenting cells than may reside in a finite area of subcutaneous tissue. It also eliminates the requirement for subcutaneous injections, the use of filgrastim, and the risk of delayed type hypersensitivity. It is also likely to generate high T cell titers faster as optimum CD8+ T cell numbers typically peak after 3 treatments, not greater than 10. Third, Lm promotes a predominant CD8+ T cell response with CD4+ cross-reactivity for T cell help. CD8+ T cells are the most effective at killing cancer cells and because Lm vectors present their antigen in the cytoplasm of the APC, those peptides are rapidly shunted to the proteasome for processing, complexed with MHC Class 1 and transported to the APC surface for presentation to predominantly CD8+ T cells. This should bring the advantage of generating more CD8+ T cells that a subcutaneous Montanide presentation of antigen peptides. Fourth, Lm vectors increase the expression of chemokine and chemokine receptors on tumors and surrounding lymph nodes. This facilitates the attraction of activated T cells to the vicinity of solid tumors. Fifth, Lm vectors decrease the relative number and suppressive function of immunosuppressive cells that may protect a tumor from T cell attack, better enabling T cell killing of cancer cells. This reduction of the immunosuppressive ability of regulatory T cells and myeloid derived suppressor cells will better enable T cells generated against these peptides to have better activity in solid tumors. Sixth, Lm vectors do not generate neutralizing antibodies. Because of this, these vectors can be administered repeatedly for extended periods of time without the loss of efficacy from neutralizing antibodies and the development of delayed-type hypersensitivity or acute hypersensitivity which may include anaphylaxis.

Lm vectors have some clinical advantages. Any side effects associated with treatment appear in the hours immediately post-infusion while the patient is still in the clinic, are almost exclusively mild-moderate and respond readily to treatment, and resolve the day of dosing without evidence of delayed onset, cumulative toxicity, or lasting sequalae. Practical advantages include the fact that there is no need to administer multiple agents and switch to alternate dosing sites for subsequent administrations.

From a manufacturing standpoint, there are several advantages. First, there is no need to manufacture the individual peptides to high concentrations and high degrees of purity. The Lm bacteria transcribe the DNA simultaneously on multiple copies of DNA plasmids inside the bacteria and secrete these peptides directly into the cytoplasm of the APC, where they are almost immediately transported to the proteasome for processing. Essentially, the peptides are manufactured by the bacteria right at the point of use for antigen processing. Second, Lm vectors are highly scalable. Once the genetic engineering is complete, the bacteria replicate themselves in broth cultures. The cultures can be scaled up to vastly reduce cost of goods. Third, there is no need to formulate in a complex carrier like Montanide or create an emulsion. Fourth, the bacteria are very stable, some more than 5 years, without worry of peptide degradation or breakdown product contamination that can lead to loss of potency of a peptide formulation.

Some of the Lm minigene constructs disclosed herein express the WT1-122A1 long, WT1-427 long, and/or WT1-331 long peptides as fusion proteins. These peptides are immunogenic in patients with hematologic malignancies or solid tumors in that the majority of patients are found to develop T cell responses to one or more of these peptides. There is a significant correlation between clinical benefit and the development of T cell responses against these peptides. The combination of these peptides provides significant cross reactivity in the major MHC Class 1 HLA haplotypes present in North America. HLA*A02, HLA*A03, HLA*B07.

Some of the Lm minigene constructs disclosed herein express the A-24-native, A-24-het-1, and/or A-24-het-2 peptides as fusion proteins. These add additional coverage for the HLA*A24 class 1 haplotype. This haplotype represents approximately 10% of the North American population, but it is much more highly represented in Asian populations. For example, some reports cite the frequency of HLA*A24 as high as 90% of people in Japan. It is also quite common in China, South Korea, and other Asian countries. The addition of the HLA*A24 peptides provide immunogenic WT1 peptides that will generate T cells against WT1 in these Asian populations and 10% of North American patients who are not covered by the WT1-122A1 long, WT1-427 long, and WT1-331 long peptides.

Use of the minigene construct approach disclosed herein for the expression of specific MHC class I binding antigenic determinants allows for the highly efficient delivery of short peptide sequences to the antigen presentation pathway of professional antigen presenting cells (pAPC). A specific advantage of the minigene technology is that it bypasses the requirement for proteasome mediated degradation of larger proteins in order to liberate short peptide sequences that can be bound and presented on MHC class I molecules. This results in a much higher efficiency of peptide-MHC class I antigen presentation on the surface of the pAPC and, therefore, a much higher level of antigen expression for the priming of antigen specific T cell responses.

### II. Recombinant PEST-Containing Fusion Polypeptides

Disclosed herein are recombinant fusion polypeptides comprising a PEST-containing peptide fused to a native (i.e., naturally occurring or unmodified) Wilms tumor protein (WT1) (i.e., a full-length, native WT1 protein) or an immunogenic (i.e., antigenic) fragment thereof. Also disclosed herein are recombinant fusion polypeptides comprising a PEST-containing peptide fused to an immunogenic (i.e., antigenic) WT1 peptide, wherein the peptide comprises, consists essentially of, or consists of the sequence set forth in SEQ ID NO: 114 or 136. Also disclosed herein are recombinant fusion polypeptides comprising a PEST-containing peptide fused to one or more of a native WT1 protein, an immunogenic fragment of a native WT1 protein, and a WT1 peptide comprising, consisting essentially of, or consisting of the sequence set forth in SEQ ID NO: 114 or 136. The PEST-containing peptide can be fused directly to the native WT1, the fragment thereof, or the WT1 peptide comprising, consisting essentially of, or consisting of SEQ ID NO: 114 or 136, or they can be linked via a peptide linker or via one or more other antigenic WT1 peptides.

Selection of, variations of, and arrangement of immunogenic (i.e., antigenic) peptides within a fusion polypeptide are discussed in detail elsewhere herein, and native WT1 peptides and immunogenic fragments thereof are discussed in more detail elsewhere herein.

The recombinant fusion polypeptides can comprise one or more tags. For example, the recombinant fusion polypeptides can comprise one or more peptide tags N-terminal and/or C-terminal to the one or more of the native WT1 protein, the immunogenic fragment of the native WT1 protein, or the WT1 peptide comprising, consisting essentially of, or consisting of the sequence set forth in SEQ ID NO: 114 or 136. A tag can be fused directly to an antigenic peptide or linked to an antigenic peptide via a linker (examples of which are disclosed elsewhere herein). Examples of tags include the following: FLAG tag; 2×FLAG tag; 3×FLAG tag; His tag, 6×His tag; and SIINFEKL tag. An exemplary SIINFEKL tag is set forth in SEQ ID NO: 16 (encoded by any one of the nucleic acids set forth in SEQ ID NOS: 1-15). An exemplary 3×FLAG tag is set forth in SEQ ID NO: 32 (encoded by any one of the nucleic acids set forth in SEQ ID NOS: 17-31). An exemplary variant 3×FLAG tag is set forth in SEQ ID NO: 99. Two or more tags can be used together, such as a 2×FLAG tag and a SIINFEKL tag, a 3×FLAG tag and a SIINFEKL tag, or a 6×His tag and a SIINFEKL tag. If two or more tags are used, they can be located anywhere within the recombinant fusion polypeptide and in any order. For example, the two tags can be at the C-terminus of the recombinant fusion polypeptide, the two tags can be at the N-terminus of the recombinant fusion polypeptide, the two tags can be located internally within the recombinant fusion polypeptide, one tag can be at the C-terminus and one tag at the N-terminus of the recombinant fusion polypeptide, one tag can be at the C-terminus and one internally within the recombinant fusion polypeptide, or one tag can be at the N-terminus and one internally within the recombinant fusion polypeptide. Other tags include chitin binding protein (CBP), maltose binding protein (MBP), glutathione-S-transferase (GST), thioredoxin (TRX), and poly(NANP). Particular recombinant fusion polypeptides comprise a C-terminal SIINFEKL tag. Such tags can allow for easy detection of the recombinant fusion protein, confirmation of secretion of the recombinant fusion protein, or for following the immunogenicity of the secreted fusion polypeptide by following immune responses to these “tag” sequence peptides. Such immune response can be monitored using a number of reagents including, for example, monoclonal antibodies and DNA or RNA probes specific for these tags.

The recombinant fusion polypeptides disclosed herein can be expressed by recombinant Listeria strains or can be expressed and isolated from other vectors and cell systems used for protein expression and isolation. Recombinant Listeria strains comprising expressing such antigenic peptides can be used, for example in immunogenic compositions comprising such recombinant Listeria and in vaccines comprising the recombinant Listeria strain and an adjuvant. Expression of one or more antigenic peptides as a fusion polypeptides with a nonhemolytic truncated form of LLO, ActA, or a PEST-like sequence in host cell systems in Listeria strains and host cell systems other than Listeria can result in enhanced immunogenicity of the antigenic peptides.

Nucleic acids encoding such recombinant fusion polypeptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames (e.g., an open reading frame encoding the recombinant fusion polypeptide and a second open reading frame encoding a metabolic enzyme). In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different polypeptide. In some nucleic acids, the codon encoding the carboxy terminus of the fusion polypeptide is followed by two stop codons to ensure termination of protein synthesis.

A. Native WT1 Proteins and Immunogenic Fragments Thereof

Wilms tumor protein (WT1, AEWS-GUD, NPHS4, WAGR, WIT-2, WT33, Wilms tumor 1) is a protein that in humans is encoded by the WT1 gene on chromosome 11p. This gene encodes a transcription factor that contains four zinc finger motifs at the C-terminus and a proline/glutamine-rich DNA-binding domain at the N-terminus. The WT1 antigen is a transcription factor that is not generally expressed in normal adult cells, but appears in a large number of cancers, as well as in certain cancer stem cells.

An exemplary human WT gene is assigned GenBank Accession No. AY245105.1. Exemplary human WT1 proteins are assigned UniProt Accession No. P19544 (SEQ ID NO: 138), NCBI Accession No. NP_001185481.1 (SEQ ID NO: 140), NCBI Accession No. NP_001185480.1 (SEQ ID NO: 141), NCBI Accession No. NP_077744.3 (SEQ ID NO: 142), NCBI Accession No. NP_077742.2 (SEQ ID NO: 143), and NCBI Accession No. NP_000369.3 (SEQ ID NO: 144). An exemplary WT1 fragment including the WT1 protein set forth in UniProt Accession No. P19544 with residues 54-68 removed is set forth in SEQ ID NO: 139. Other exemplary human WT1 proteins or fragments thereof are set forth in SEQ ID NOS: 145-148. Exemplary WT1 protein homologs from other species include UniProt Accession Nos. P22561 (mouse), P49952 (rat), O62651 (pig), and B7ZSG3 (Xenopus).

A native WT1 protein is a WT1 protein that is naturally occurring (e.g., without heteroclitic mutations generated). Nucleic acids encoding full-length native WT1 proteins or immunogenic/antigenic fragments of native WT1 proteins can be used. Fragments of native WT1 proteins include native WT1 protein sequences in which one or more of a C-terminal segment has been removed, an N-terminal segment has been removed, or one or more internal fragments have been removed. For example, a fragment can be an N-terminal fragment of WT1, a C-terminal fragment of WT1, an internal fragment of WT1, a WT1 protein with one or more internal fragments removed, and so forth. An example of a WT1 protein fragment with an internal fragment removed is set forth in SEQ ID NO: 139. An example of an internal fragment of a native WT1 protein (i.e., a fragment of a native WT1 protein with C-terminal and N-terminal sequence removed) is set forth in SEQ ID NO: 95. In one example, a fragment of a native WT1 protein included in the fusion polypeptide comprises, consists essentially of, or consists of the sequence set forth in SEQ ID NO: 95 or SEQ ID NO: 139. Yet other examples of suitable native WT1 peptides are disclosed elsewhere herein.

The fusion polypeptide can include the PEST-containing peptide fused to one antigenic or immunogenic WT1 peptides selected from a native Wilms tumor protein (WT1), an immunogenic fragment of the native WT1, and a WT1 peptide consisting, consisting essentially of, or comprising the sequence set forth in SEQ ID NO: 114 or SEQ ID NO: 136. Alternatively, the fusion polypeptide can include the PEST-containing peptide fused to two or more antigenic or immunogenic WT1 peptides selected from a native Wilms tumor protein (WT1), an immunogenic fragment of the native WT1, and a WT1 peptide consisting, consisting essentially of, or comprising the sequence set forth in SEQ ID NO: 114 or SEQ ID NO: 136. For example, the fusion polypeptide can comprise 2-20, 2-15, 2-10, 2-5, 2-4, 2, 3, 4, 5, 6, 7, 8, 9, 10, 11, 12, 13, 14, 15, 16, 17, 18, 18, or 20 antigenic WT1 peptides. Two or more or all of the antigenic WT1 peptides can be the same, or two or more or all of the antigenic WT1 peptides can be different (e.g., each antigenic WT1 peptide can be different). A first antigenic peptide is different from a second antigenic peptide if it includes a single amino acid that is not in the second antigenic peptide. Each antigenic WT1 peptide can be of any length sufficient to induce an immune response, and each antigenic WT1 peptide can be the same length or the antigenic peptides can have different lengths. For example, an antigenic WT1 peptide disclosed herein can be between about 8-600, 8-500, 8-450, 8-400, 8-350, 8-300, 8-250, 8-200, 8-150, 8-100, 8-90, 8-80, 8-70, 8-60, 8-50, 8-40, 8-30, 8-20, 8-15, 8-12, or 8-10 amino acids in length. Alternatively, an antigenic WT1 peptide can be, for example, no more than about 8, 9, 10, 12, 15, 20, 30, 40, 50, 60, 70, 80, 90, 100, 150, 200, 250, 300, 350, 400, 450, 500, 550, or 600 amino acids in length, or an antigenic WT1 peptide can be, for example, at least about 8, 9, 10, 12, 15, 20, 30, 40, 50, 60, 70, 80, 90, 100, 150, 200, 250, 300, 350, 400, 450, 500, 550, or 600 amino acids in length.

Each antigenic peptide can also be hydrophilic or can score up to or below a certain hydropathy threshold, which can be predictive of secretability in Listeria monocytogenes or another bacteria of interest. For example, antigenic peptides can be scored by a Kyte and Doolittle hydropathy index 21 amino acid window, and all scoring above a cutoff (around 1.6) can be excluded as they are unlikely to be secretable by Listeria monocytogenes. Likewise, the combination of antigenic peptides or the fusion polypeptide can be hydrophilic or can score up to or below a certain hydropathy threshold, which can be predictive of secretability in Listeria monocytogenes or another bacteria of interest.

If the fusion polypeptide comprises two or more antigenic peptides, the antigenic peptides can be linked together in any manner. For example, the antigenic peptides can be fused directly to each other with no intervening sequence. Alternatively, the antigenic peptides can be linked to each other indirectly via one or more linkers, such as peptide linkers. In some cases, some pairs of adjacent antigenic peptides can be fused directly to each other, and other pairs of antigenic peptides can be linked to each other indirectly via one or more linkers. The same linker can be used between each pair of adjacent antigenic peptides, or any number of different linkers can be used between different pairs of adjacent antigenic peptides. In addition, one linker can be used between a pair of adjacent antigenic peptides, or multiple linkers can be used between a pair of adjacent antigenic peptides.

Any suitable sequence can be used for a peptide linker. As an example, a linker sequence may be, for example, from 1 to about 50 amino acids in length. Some linkers may be hydrophilic. The linkers can serve varying purposes. For example, the linkers can serve to increase bacterial secretion, to facilitate antigen processing, to increase flexibility of the fusion polypeptide, to increase rigidity of the fusion polypeptide, or any other purpose. In some cases, different amino acid linker sequences are distributed between the antigenic peptides or different nucleic acids encoding the same amino acid linker sequence are distributed between the antigenic peptides (e.g., SEQ ID NOS: 84-94) in order to minimize repeats. This can also serve to reduce secondary structures, thereby allowing efficient transcription, translation, secretion, maintenance, or stabilization of the nucleic acid (e.g., plasmid) encoding the fusion polypeptide within a Lm recombinant vector strain population. Other suitable peptide linker sequences may be chosen, for example, based on one or more of the following factors: (1) their ability to adopt a flexible extended conformation; (2) their inability to adopt a secondary structure that could interact with functional epitopes on the antigenic peptides; and (3) the lack of hydrophobic or charged residues that might react with the functional epitopes. For example, peptide linker sequences may contain Gly, Asn and Ser residues. Other near neutral amino acids, such as Thr and Ala may also be used in the linker sequence. Amino acid sequences which may be usefully employed as linkers include those disclosed in Maratea et al. (1985) Gene 40:39-46; Murphy et al. (1986) Proc Natl Acad Sci USA 83:8258-8262; U.S. Pat. Nos. 4,935,233; and 4,751,180, each of which is herein incorporated by reference in its entirety for all purposes. Specific examples of linkers include those in the following table (each of which can be used by itself as a linker, in a linker comprising repeats of the sequence, or in a linker further comprising one or more of the other sequences in the table), although others can also be envisioned (see, e.g., Reddy Chichili et al. (2013) Protein Science 22:153-167, herein incorporated by reference in its entirety for all purposes). Unless specified, “n” represents an undetermined number of repeats in the listed linker.

| Peptide | | SEQ | Hypothetical |
|---|---|---|---|
| Linker | Example | ID NO: | Purpose |
| (GAS)ₙ | GASGAS | 33 | Flexibility |
| (GSA)ₙ | GSAGSA | 34 | Flexibility |
| (G)ₙ; | GGGG | 35 | Flexibility |
| n = 4-8 | | | |
| (GGGGS)ₙ; | GGGGS | 36 | Flexibility |
| n = 1-3 | | | |
| VGKGGSGG | VGKGGSGG | 37 | Flexibility |
| (PAPAP)ₙ | PAPAP | 38 | Rigidity |
| (EAAAK)ₙ; | EAAAK | 39 | Rigidity |
| n = 1-3 | | | |
| (AYL)ₙ | AYLAYL | 40 | Antigen |
| | | | Processing |
| (LRA)ₙ | LRALRA | 41 | Antigen |
| | | | Processing |
| (RLRA)ₙ | RLRA | 42 | Antigen |
| | | | Processing |

B. PEST-Containing Peptides

The recombinant fusion proteins disclosed herein comprise a PEST-containing peptide. The PEST-containing peptide may at the amino terminal (N-terminal) end of the fusion polypeptide (i.e., N-terminal to the antigenic peptides), may be at the carboxy terminal (C-terminal) end of the fusion polypeptide (i.e., C-terminal to the antigenic peptides), or may be embedded within the antigenic peptides. In some recombinant Listeria strains and methods, a PEST containing peptide is not part of and is separate from the fusion polypeptide. Fusion of an antigenic peptides to a PEST-like sequence, such as an LLO peptide, can enhance the immunogenicity of the antigenic peptides and can increase cell-mediated and antitumor immune responses (i.e., increase cell-mediated and anti-tumor immunity). See, e.g., Singh et al. (2005) J Immunol 175(6):3663-3673, herein incorporated by reference in its entirety for all purposes.

A PEST-containing peptide is one that comprises a PEST sequence or a PEST-like sequence. PEST sequences in eukaryotic proteins have long been identified. For example, proteins containing amino acid sequences that are rich in prolines (P), glutamic acids (E), serines (S) and threonines (T) (PEST), generally, but not always, flanked by clusters containing several positively charged amino acids, have rapid intracellular half-lives (Rogers et al. (1986) Science 234:364-369, herein incorporated by reference in its entirety for all purposes). Further, it has been reported that these sequences target the protein to the ubiquitin-proteosome pathway for degradation (Rechsteiner and Rogers (1996) Trends Biochem. Sci. 21:267-271, herein incorporated by reference in its entirety for all purposes). This pathway is also used by eukaryotic cells to generate immunogenic peptides that bind to MHC class I and it has been hypothesized that PEST sequences are abundant among eukaryotic proteins that give rise to immunogenic peptides (Realini et al. (1994) FEBS Lett. 348:109-113, herein incorporated by reference in its entirety for all purposes). Prokaryotic proteins do not normally contain PEST sequences because they do not have this enzymatic pathway. However, a PEST-like sequence rich in the amino acids proline (P), glutamic acid (E), serine (S) and threonine (T) has been reported at the amino terminus of LLO and has been reported to be essential for L. monocytogenes pathogenicity (Decatur and Portnoy (2000) Science 290:992-995, herein incorporated by reference in its entirety for all purposes). The presence of this PEST-like sequence in LLO targets the protein for destruction by proteolytic machinery of the host cell so that once the LLO has served its function and facilitated the escape of L. monocytogenes from the phagosomal or phagolysosomal vacuole, it is destroyed before it can damage the cells.

Identification of PEST and PEST-like sequences is well known in the art and is described, for example, in Rogers et al. (1986) Science 234(4774):364-378 and in Rechsteiner and Rogers (1996) Trends Biochem. Sci. 21:267-271, each of which is herein incorporated by reference in its entirety for all purposes. A PEST or PEST-like sequence can be identified using the PEST-find program. For example, a PEST-like sequence can be a region rich in proline (P), glutamic acid (E), serine (S), and threonine (T) residues. Optionally, the PEST-like sequence can be flanked by one or more clusters containing several positively charged amino acids. For example, a PEST-like sequence can be defined as a hydrophilic stretch of at least 12 amino acids in length with a high local concentration of proline (P), aspartate (D), glutamate (E), serine (S), and/or threonine (T) residues. In some cases, a PEST-like sequence contains no positively charged amino acids, namely arginine (R), histidine (H), and lysine (K). Some PEST-like sequences can contain one or more internal phosphorylation sites, and phosphorylation at these sites precedes protein degradation.

In one example, the PEST-like sequence fits an algorithm disclosed in Rogers et al. In another example, the PEST-like sequence fits an algorithm disclosed in Rechsteiner and Rogers. PEST-like sequences can also be identified by an initial scan for positively charged amino acids R, H, and K within the specified protein sequence. All amino acids between the positively charged flanks are counted, and only those motifs containing a number of amino acids equal to or higher than the window-size parameter are considered further. Optionally, a PEST-like sequence must contain at least one P, at least one D or E, and at least one S or T.

The quality of a PEST motif can be refined by means of a scoring parameter based on the local enrichment of critical amino acids as well as the motifs hydrophobicity. Enrichment of D, E, P, S, and T is expressed in mass percent (w/w) and corrected for one equivalent of D or E, one 1 of P, and one of S or T. Calculation of hydrophobicity can also follow in principle the method of Kyte and Doolittle (1982) J. Mol. Biol. 157:105, herein incorporated by reference in its entirety for all purposes. For simplified calculations, Kyte-Doolittle hydropathy indices, which originally ranged from −4.5 for arginine to +4.5 for isoleucine, are converted to positive integers, using the following linear transformation, which yielded values from 0 for arginine to 90 for isoleucine: Hydropathy index=10*Kyte-Doolittle hydropathy index+45.

A potential PEST motif's hydrophobicity can also be calculated as the sum over the products of mole percent and hydrophobicity index for each amino acid species. The desired PEST score is obtained as combination of local enrichment term and hydrophobicity term as expressed by the following equation: PEST score=0.55*DEPST−0.5*hydrophobicity index.

Thus, a PEST-containing peptide can refer to a peptide having a score of at least +5 using the above algorithm. Alternatively, it can refer to a peptide having a score of at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 32, at least 35, at least 38, at least 40, or at least 45.

Any other available methods or algorithms known in the art can also be used to identify PEST-like sequences. See, e.g., the CaSPredictor (Garay-Malpartida et al. (2005) Bioinformatics 21 Suppl 1:i169-76, herein incorporated by reference in its entirety for all purposes). Another method that can be used is the following: a PEST index is calculated for each stretch of appropriate length (e.g. a 30-35 amino acid stretch) by assigning a value of one to the amino acids Ser, Thr, Pro, Glu, Asp, Asn, or Gln. The coefficient value (CV) for each of the PEST residues is one and the CV for each of the other AA (non-PEST) is zero.

Examples of PEST-like amino acid sequences are those set forth in SEQ ID NOS: 43-51. One example of a PEST-like sequence is KENSISSMAPPASPPASPKTPIEKKHADEIDK (SEQ ID NO: 43). Another example of a PEST-like sequence is KENSISSMAPPASPPASPK (SEQ ID NO: 44). However, any PEST or PEST-like amino acid sequence can be used. PEST sequence peptides are known and are described, for example, in U.S. Pat. Nos. 7,635,479; 7,665,238; and US 2014/0186387, each of which is herein incorporated by reference in its entirety for all purposes.

The PEST-like sequence can be from a Listeria species, such as from Listeria monocytogenes. For example, the Listeria monocytogenes ActA protein contains at least four such sequences (SEQ ID NOS: 45-48), any of which are suitable for use in the compositions and methods disclosed herein. Other similar PEST-like sequences include SEQ ID NOS: 52-54. Streptolysin O proteins from Streptococcus sp. also contain a PEST sequence. For example, Streptococcus pyogenes streptolysin O comprises the PEST sequence KQNTASTETTTTNEQPK (SEQ ID NO: 49) at amino acids 35-51 and Streptococcus equisimilis streptolysin O comprises the PEST-like sequence KQNTANTETTTTNEQPK (SEQ ID NO: 50) at amino acids 38-54. Another example of a PEST-like sequence is from Listeria seeligeri cytolysin, encoded by the lso gene: RSEVTISPAETPESPPATP (e.g., SEQ ID NO: 51).

Alternatively, the PEST-like sequence can be derived from other prokaryotic organisms. Other prokaryotic organisms wherein PEST-like amino acid sequences would be expected include, for example, other Listeria species.

(I) Listeriolysin O (LLO)

One example of a PEST-containing peptide that can be utilized in the compositions and methods disclosed herein is a listeriolysin O (LLO) peptide. An example of an LLO protein is the protein assigned GenBank Accession No. P13128 (SEQ ID NO: 55; nucleic acid sequence is set forth in GenBank Accession No. X15127). SEQ ID NO: 55 is a proprotein including a signal sequence. The first 25 amino acids of the proprotein is the signal sequence and is cleaved from LLO when it is secreted by the bacterium, thereby resulting in the full-length active LLO protein of 504 amino acids without the signal sequence. An LLO peptide disclosed herein can comprise the signal sequence or can comprise a peptide that does not include the signal sequence. Exemplary LLO proteins that can be used comprise, consist essentially of, or consist of the sequence set forth in SEQ ID NO: 55 or homologues, variants, isoforms, analogs, fragments, fragments of homologues, fragments of variants, fragments of analogs, and fragments of isoforms of SEQ ID NO: 55. Any sequence that encodes a fragment of an LLO protein or a homologue, variant, isoform, analog, fragment of a homologue, fragment of a variant, or fragment of an analog of an LLO protein can be used. A homologous LLO protein can have a sequence identity with a reference LLO protein, for example, of greater than 70%, 72%, 75%, 78%, 80%, 82%, 83%, 85%, 87%, 88%, 90%, 92%, 93%, 95%, 96%, 97%, 98%, or 99%.

Another example of an LLO protein is set forth in SEQ ID NO: 56. LLO proteins that can be used can comprise, consist essentially of, or consist of the sequence set forth in SEQ ID NO: 56 or homologues, variants, isoforms, analogs, fragments, fragments of homologues, fragments of variants, fragments of analogs, and fragments of isoforms of SEQ ID NO: 56.

Another example of an LLO protein is an LLO protein from the Listeria monocytogenes 10403S strain, as set forth in GenBank Accession No.: ZP_01942330 or EBA21833, or as encoded by the nucleic acid sequence as set forth in GenBank Accession No.: NZ_AARZ01000015 or AARZ01000015.1. Another example of an LLO protein is an LLO protein from the Listeria monocytogenes 4b F2365 strain (see, e.g., GenBank Accession No.: YP_012823), EGD-e strain (see, e.g., GenBank Accession No.: NP_463733), or any other strain of Listeria monocytogenes. Yet another example of an LLO protein is an LLO protein from Flavobacteriales bacterium HTCC2170 (see, e.g., GenBank Accession No.: ZP_01106747 or EAR01433, or encoded by GenBank Accession No.: NZ_AAOC01000003). LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

Proteins that are homologous to LLO, or homologues, variants, isoforms, analogs, fragments, fragments of homologues, fragments of variants, fragments of analogs, and fragments of isoforms thereof, can also be used. One such example is alveolysin, which can be found, for example, in Paenibacillus alvei (see, e.g., GenBank Accession No.: P23564 or AAA22224, or encoded by GenBank Accession No.: M62709). Other such homologous proteins are known.

The LLO peptide can be a full-length LLO protein or a truncated LLO protein or LLO fragment. Likewise, the LLO peptide can be one that retains one or more functionalities of a native LLO protein or lacks one or more functionalities of a native LLO protein. For example, the retained LLO functionality can be allowing a bacteria (e.g., Listeria) to escape from a phagosome or phagolysosome, or enhancing the immunogenicity of a peptide to which it is fused. The retained functionality can also be hemolytic function or antigenic function. Alternatively, the LLO peptide can be a non-hemolytic LLO. Other functions of LLO are known, as are methods and assays for evaluating LLO functionality.

An LLO fragment can be a PEST-like sequence or can comprise a PEST-like sequence. LLO fragments can comprise one or more of an internal deletion, a truncation from the C-terminal end, and a truncation from the N-terminal end. In some cases, an LLO fragment can comprise more than one internal deletion. Other LLO peptides can be full-length LLO proteins with one or more mutations.

Some LLO proteins or fragments have reduced hemolytic activity relative to wild type LLO or are non-hemolytic fragments. For example, an LLO protein can be rendered non-hemolytic by deletion or mutation of the activation domain at the carboxy terminus, by deletion or mutation of cysteine 484, or by deletion or mutation at another location.

Other LLO proteins are rendered non-hemolytic by a deletion or mutation of the cholesterol binding domain (CBD) as detailed in U.S. Pat. No. 8,771,702, herein incorporated by reference in its entirety for all purposes. The mutations can comprise, for example, a substitution or a deletion. The entire CBD can be mutated, portions of the CBD can be mutated, or specific residues within the CBD can be mutated. For example, the LLO protein can comprise a mutation of one or more of residues C484, W491, and W492 (e.g., C484, W491, W492, C484 and W491, C484 and W492, W491 and W492, or all three residues) of SEQ ID NO: 55 or corresponding residues when optimally aligned with SEQ ID NO: 55 (e.g., a corresponding cysteine or tryptophan residue). As an example, a mutant LLO protein can be created wherein residues C484, W491, and W492 of LLO are substituted with alanine residues, which will substantially reduce hemolytic activity relative to wild type LLO. The mutant LLO protein with C484A, W491A, and W492A mutations is termed “mutLLO.”

As another example, a mutant LLO protein can be created with an internal deletion comprising the cholesterol-binding domain. The sequence of the cholesterol-binding domain of SEQ ID NO: 55 set forth in SEQ ID NO: 74. For example, the internal deletion can be a 1-11 amino acid deletion, an 11-50 amino acid deletion, or longer. Likewise, the mutated region can be 1-11 amino acids, 11-50 amino acids, or longer (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). For example, a mutated region consisting of residues 470-500, 470-510, or 480-500 of SEQ ID NO: 55 will result in a deleted sequence comprising the CBD (residues 483-493 of SEQ ID NO: 55). However, the mutated region can also be a fragment of the CBD or can overlap with a portion of the CBD. For example, the mutated region can consist of residues 470-490, 480-488, 485-490, 486-488, 490-500, or 486-510 of SEQ ID NO: 55. For example, a fragment of the CBD (residues 484-492) can be replaced with a heterologous sequence, which will substantially reduce hemolytic activity relative to wild type LLO. For example, the CBD (ECTGLAWEWWR; SEQ ID NO: 74) can be replaced with a CTL epitope from the antigen NY-ESO-1 (ESLLMWITQCR; SEQ ID NO: 75), which contains the HLA-A2 restricted epitope 157-165 from NY-ESO-1. The resulting LLO is termed “ctLLO.”

In some mutated LLO proteins, the mutated region can be replaced by a heterologous sequence. For example, the mutated region can be replaced by an equal number of heterologous amino acids, a smaller number of heterologous amino acids, or a larger number of amino acids (e.g., 1-50, 1-11, 2-11, 3-11, 4-11, 5-11, 6-11, 7-11, 8-11, 9-11, 10-11, 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 12-50, 11-15, 11-20, 11-25, 11-30, 11-35, 11-40, 11-50, 11-60, 11-70, 11-80, 11-90, 11-100, 11-150, 15-20, 15-25, 15-30, 15-35, 15-40, 15-50, 15-60, 15-70, 15-80, 15-90, 15-100, 15-150, 20-25, 20-30, 20-35, 20-40, 20-50, 20-60, 20-70, 20-80, 20-90, 20-100, 20-150, 30-35, 30-40, 30-60, 30-70, 30-80, 30-90, 30-100, or 30-150 amino acids). Other mutated LLO proteins have one or more point mutations (e.g., a point mutation of 1 residue, 2 residues, 3 residues, or more). The mutated residues can be contiguous or not contiguous.

In one example embodiment, an LLO peptide may have a deletion in the signal sequence and a mutation or substitution in the CBD.

Some LLO peptides are N-terminal LLO fragments (i.e., LLO proteins with a C-terminal deletion). Some LLO peptides are at least 494, 489, 492, 493, 500, 505, 510, 515, 520, or 525 amino acids in length or 492-528 amino acids in length. For example, the LLO fragment can consist of about the first 440 or 441 amino acids of an LLO protein (e.g., the first 441 amino acids of SEQ ID NO: 55 or 56, or a corresponding fragment of another LLO protein when optimally aligned with SEQ ID NO: 55 or 56). Other N-terminal LLO fragments can consist of the first 420 amino acids of an LLO protein (e.g., the first 420 amino acids of SEQ ID NO: 55 or 56, or a corresponding fragment of another LLO protein when optimally aligned with SEQ ID NO: 55 or 56). Other N-terminal fragments can consist of about amino acids 20-442 of an LLO protein (e.g., amino acids 20-442 of SEQ ID NO: 55 or 56, or a corresponding fragment of another LLO protein when optimally aligned with SEQ ID NO: 55 or 56). Other N-terminal LLO fragments comprise any ALLO without the activation domain comprising cysteine 484, and in particular without cysteine 484. For example, the N-terminal LLO fragment can correspond to the first 425, 400, 375, 350, 325, 300, 275, 250, 225, 200, 175, 150, 125, 100, 75, 50, or 25 amino acids of an LLO protein (e.g., the first 425, 400, 375, 350, 325, 300, 275, 250, 225, 200, 175, 150, 125, 100, 75, 50, or 25 amino acids of SEQ ID NO: 55 or 56, or a corresponding fragment of another LLO protein when optimally aligned with SEQ ID NO: 55 or 56). Preferably, the fragment comprises one or more PEST-like sequences. LLO fragments and truncated LLO proteins can contain residues of a homologous LLO protein that correspond to any one of the above specific amino acid ranges. The residue numbers need not correspond exactly with the residue numbers enumerated above (e.g., if the homologous LLO protein has an insertion or deletion relative to a specific LLO protein disclosed herein). Examples of N-terminal LLO fragments include SEQ ID NOS: 57, 58, and 59. LLO proteins that can be used comprise, consist essentially of, or consist of the sequence set forth in SEQ ID NO: 57, 58, or 59 or homologues, variants, isoforms, analogs, fragments, fragments of homologues, fragments of variants, fragments of analogs, and fragments of isoforms of SEQ ID NO: 57, 58, or 59. In some compositions and methods, the N-terminal LLO fragment set forth in SEQ ID NO: 59 is used. An example of a nucleic acid encoding the N-terminal LLO fragment set forth in SEQ ID NO: 59 is SEQ ID NO: 60.

(2) ActA

Another example of a PEST-containing peptide that can be utilized in the compositions and methods disclosed herein is an ActA peptide. ActA is a surface-associated protein and acts as a scaffold in infected host cells to facilitate the polymerization, assembly, and activation of host actin polymers in order to propel a Listeria monocytogenes through the cytoplasm. Shortly after entry into the mammalian cell cytosol, L. monocytogenes induces the polymerization of host actin filaments and uses the force generated by actin polymerization to move, first intracellularly and then from cell to cell. ActA is responsible for mediating actin nucleation and actin-based motility. The ActA protein provides multiple binding sites for host cytoskeletal components, thereby acting as a scaffold to assemble the cellular actin polymerization machinery. The N-terminus of ActA binds to monomeric actin and acts as a constitutively active nucleation promoting factor by stimulating the intrinsic actin nucleation activity. The actA and hly genes are both members of the 10-kb gene cluster regulated by the transcriptional activator PrfA, and actA is upregulated approximately 226-fold in the mammalian cytosol. Any sequence that encodes an ActA protein or a homologue, variant, isoform, analog, fragment of a homologue, fragment of a variant, or fragment of an analog of an ActA protein can be used. A homologous ActA protein can have a sequence identity with a reference ActA protein, for example, of greater than 70%, 72%, 75%, 78%, 80%, 82%, 83%, 85%, 87%, 88%, 90%, 92%, 93%, 95%, 96%, 97%, 98%, or 99%.

One example of an ActA protein comprises, consists essentially of, or consists of the sequence set forth in SEQ ID NO: 61. Another example of an ActA protein comprises, consists essentially of, or consists of the sequence set forth in SEQ ID NO: 62. The first 29 amino acid of the proprotein corresponding to either of these sequences are the signal sequence and are cleaved from ActA protein when it is secreted by the bacterium. An ActA peptide can comprise the signal sequence (e.g., amino acids 1-29 of SEQ ID NO: 61 or 62), or can comprise a peptide that does not include the signal sequence. Other examples of ActA proteins comprise, consist essentially of, or consist of homologues, variants, isoforms, analogs, fragments, fragments of homologues, fragments of isoforms, or fragments of analogs of SEQ ID NO: 61 or 62.

Another example of an ActA protein is an ActA protein from the Listeria monocytogenes 10403S strain (GenBank Accession No.: DQ054585) the NICPBP 54002 strain (GenBank Accession No.: EU394959), the S3 strain (GenBank Accession No.: EU394960), NCTC 5348 strain (GenBank Accession No.: EU394961), NICPBP 54006 strain (GenBank Accession No.: EU394962), M7 strain (GenBank Accession No.: EU394963), S19 strain (GenBank Accession No.: EU394964), or any other strain of Listeria monocytogenes. LLO proteins that can be used can comprise, consist essentially of, or consist of any of the above LLO proteins or homologues, variants, isoforms, analogs, fragments, fragments of homologues, fragments of variants, fragments of analogs, and fragments of isoforms of the above LLO proteins.

ActA peptides can be full-length ActA proteins or truncated ActA proteins or ActA fragments (e.g., N-terminal ActA fragments in which a C-terminal portion is removed). Preferably, truncated ActA proteins comprise at least one PEST sequence (e.g., more than one PEST sequence). In addition, truncated ActA proteins can optionally comprise an ActA signal peptide. Examples of PEST-like sequences contained in truncated ActA proteins include SEQ ID NOS: 45-48. Some such truncated ActA proteins comprise at least two of the PEST-like sequences set forth in SEQ ID NOS: 45-48 or homologs thereof, at least three of the PEST-like sequences set forth in SEQ ID NOS: 45-48 or homologs thereof, or all four of the PEST-like sequences set forth in SEQ ID NOS: 45-48 or homologs thereof. Examples of truncated ActA proteins include those comprising, consisting essentially of, or consisting of about residues 30-122, about residues 30-229, about residues 30-332, about residues 30-200, or about residues 30-399 of a full length ActA protein sequence (e.g., SEQ ID NO: 62). Other examples of truncated ActA proteins include those comprising, consisting essentially of, or consisting of about the first 50, 100, 150, 200, 233, 250, 300, 390, 400, or 418 residues of a full length ActA protein sequence (e.g., SEQ ID NO: 62). Other examples of truncated ActA proteins include those comprising, consisting essentially of, or consisting of about residues 200-300 or residues 300-400 of a full length ActA protein sequence (e.g., SEQ ID NO: 62). For example, the truncated ActA consists of the first 390 amino acids of the wild type ActA protein as described in U.S. Pat. No. 7,655,238, herein incorporated by reference in its entirety for all purposes. As another example, the truncated ActA can be an ActA-N100 or a modified version thereof (referred to as ActA-N100*) in which a PEST motif has been deleted and containing the nonconservative QDNKR (SEQ ID NO: 73) substitution as described in US 2014/0186387, herein incorporated by references in its entirety for all purposes. Alternatively, truncated ActA proteins can contain residues of a homologous ActA protein that corresponds to one of the above amino acid ranges or the amino acid ranges of any of the ActA peptides disclosed herein. The residue numbers need not correspond exactly with the residue numbers enumerated herein (e.g., if the homologous ActA protein has an insertion or deletion, relative to an ActA protein utilized herein, then the residue numbers can be adjusted accordingly).

Examples of truncated ActA proteins include, for example, proteins comprising, consisting essentially of, or consisting of the sequence set forth in SEQ ID NO: 63, 64, 65, or 66 or homologues, variants, isoforms, analogs, fragments of variants, fragments of isoforms, or fragments of analogs of SEQ ID NO: 63, 64, 65, or 66. SEQ ID NO: 63 referred to as ActA/PEST1 and consists of amino acids 30-122 of the full length ActA sequence set forth in SEQ ID NO: 62. SEQ ID NO: 64 is referred to as ActA/PEST2 or LA229 and consists of amino acids 30-229 of the full length ActA sequence set forth in the full-length ActA sequence set forth in SEQ ID NO: 62. SEQ ID NO: 65 is referred to as ActA/PEST3 and consists of amino acids 30-332 of the full-length ActA sequence set forth in SEQ ID NO: 62. SEQ ID NO: 66 is referred to as ActA/PEST4 and consists of amino acids 30-399 of the full-length ActA sequence set forth in SEQ ID NO: 62. As a specific example, the truncated ActA protein consisting of the sequence set forth in SEQ ID NO: 64 can be used.

Examples of truncated ActA proteins include, for example, proteins comprising, consisting essentially of, or consisting of the sequence set forth in SEQ ID NO: 67, 69, 70, or 72 or homologues, variants, isoforms, analogs, fragments of variants, fragments of isoforms, or fragments of analogs of SEQ ID NO: 67, 69, 70, or 72. As a specific example, the truncated ActA protein consisting of the sequence set forth in SEQ ID NO: 67 (encoded by the nucleic acid set forth in SEQ ID NO: 68) can be used. As another specific example, the truncated ActA protein consisting of the sequence set forth in SEQ ID NO: 70 (encoded by the nucleic acid set forth in SEQ ID NO: 71) can be used. SEQ ID NO: 71 is the first 1170 nucleotides encoding ActA in the Listeria monocytogenes 10403S strain. In some cases, the ActA fragment can be fused to a heterologous signal peptide. For example, SEQ ID NO: 72 sets forth an ActA fragment fused to an Hly signal peptide.

C. Generating Immunotherapy Constructs Encoding Recombinant Fusion Polypeptides

Also provided herein are methods for generating immunotherapy constructs encoding or compositions comprising the recombinant fusion polypeptides disclosed herein. For example, such methods can comprise selecting and designing antigenic or immunogenic peptides to include in the immunotherapy construct (and, for example, testing the hydropathy of the each antigenic peptide, and modifying or deselecting an antigenic peptide if it scores above a selected hydropathy index threshold value), designing one or more fusion polypeptides comprising each of the selected antigenic peptides, and generating a nucleic acid construct encoding the fusion polypeptide.

The antigenic peptides can be screened for hydrophobicity or hydrophilicity. Antigenic peptides can be selected, for example, if they are hydrophilic or if they score up to or below a certain hydropathy threshold, which can be predictive of secretability in a particular bacteria of interest (e.g., Listeria monocytogenes). For example, antigenic peptides can be scored by Kyte and Doolittle hydropathy index with a 21 amino acid window, all scoring above cutoff (around 1.6) are excluded as they are unlikely to be secretable by Listeria monocytogenes. See, e.g., Kyte-Doolittle (1982) J Mol Biol 157(1):105-132; herein incorporated by reference in its entirety for all purposes. Alternatively, an antigenic peptide scoring about a selected cutoff can be altered (e.g., changing the length of the antigenic peptide). Other sliding window sizes that can be used include, for example, 9, 11, 13, 15, 17, 19, 21, 23, 25, 27 or more amino acids. For example, the sliding window size can be 9-11 amino acids, 11-13 amino acids, 13-15 amino acids, 15-17 amino acids, 17-19 amino acids, 19-21 amino acids, 21-23 amino acids, 23-25 amino acids, or 25-27 amino acids. Other cutoffs that can be used include, for example, the following ranges 1.2-1.4, 1.4-1.6, 1.6-1.8, 1.8-2.0, 2.0-2.2 2.2-2.5, 2.5-3.0, 3.0-3.5, 3.5-4.0, or 4.0-4.5, or the cutoff can be 1.4, 1.5, 1.7, 1.8, 1.9, 2.0, 2.1, 2.2, 2.3, 2.3, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, or 4.5. The cutoff can vary, for example, depending on the genus or species of the bacteria being used to deliver the fusion polypeptide.

Other suitable hydropathy plots or other appropriate scales include, for example, those reported in Rose et al. (1993) Annu Rev Biomol Struct 22:381-415; Biswas et al. (2003) Journal of Chromatography A 1000:637-655; Eisenberg (1984) Ann Rev Biochem 53:595-623; Abraham and Leo (1987) Proteins: Structure, Function and Genetics 2:130-152; Sweet and Eisenberg (1983) Mol Biol 171:479-488; Bull and Breese (1974) Arch Biochem Biophys 161:665-670; Guy (1985) Biophys J 47:61-70; Miyazawa et al. (1985) Macromolecules 18:534-552; Roseman (1988) J Mol Biol 200:513-522; Wolfenden et al. (1981) Biochemistry 20:849-855; Wilson (1981) Biochem J 199:31-41; Cowan and Whittaker (1990) Peptide Research 3:75-80; Aboderin (1971) Int J Biochem 2:537-544; Eisenberg et al. (1984) J Mol Biol 179:125-142; Hopp and Woods (1981) Proc Natl Acad Sci USA 78:3824-3828; Manavalan and Ponnuswamy (1978) Nature 275:673-674; Black and Mould (1991) Anal Biochem 193:72-82; Fauchere and Pliska (1983) Eur J Med Chem 18:369-375; Janin (1979) Nature 277:491-492; Rao and Argos (1986) Biochim Biophys Acta 869:197-214; Tanford (1962) Am Chem Soc 84:4240-4274; Welling et al. (1985) FEBS Lett 188:215-218; Parker et al. (1986) Biochemistry 25:5425-5431; and Cowan and Whittaker (1990) Peptide Research 3:75-80, each of which is herein incorporated by reference in its entirety for all purposes.

Optionally, the antigenic peptides can be scored for their ability to bind to the subject human leukocyte antigen (HLA) type (for example by using the Immune Epitope Database (IED) available at www.iedb.org, which includes netMHCpan, ANN, SMMPMBEC. SMM, CombLib_Sidney2008, PickPocket, and netMHCcons) and ranked by best MHC binding score from each antigenic peptide. Other sources include TEpredict (tepredict.sourceforge.net/help.html) or other available MHC binding measurement scales. Cutoffs may be different for different expression vectors such as Salmonella.

Optionally, the antigenic peptides can be screened for immunosuppressive epitopes (e.g., T-reg epitopes, IL-10-inducing T helper epitopes, and so forth) to deselect antigenic peptides or to avoid immunosuppressive influences.

Optionally, a predicative algorithm for immunogenicity of the epitopes can be used to screen the antigenic peptides. However, these algorithms are at best 20% accurate in predicting which peptide will generate a T cell response. Alternatively, no screening/predictive algorithms are used. Alternatively, the antigenic peptides can be screened for immunogenicity. For example, this can comprise contacting one or more T cells with an antigenic peptide, and analyzing for an immunogenic T cell response, wherein an immunogenic T cell response identifies the peptide as an immunogenic peptide. This can also comprise using an immunogenic assay to measure secretion of at least one of CD25, CD44, or CD69 or to measure secretion of a cytokine selected from the group comprising IFN-γ, TNF-α, IL-1, and IL-2 upon contacting the one or more T cells with the peptide, wherein increased secretion identifies the peptide as comprising one or more T cell epitopes.

The selected antigenic peptides can be arranged into one or more candidate orders for a potential fusion polypeptide. If there are more usable antigenic peptides than can fit into a single plasmid, different antigenic peptides can be assigned priority ranks as needed/desired and/or split up into different fusion polypeptides (e.g., for inclusion in different recombinant Listeria strains). Priority rank can be determined by factors such as relative size, priority of transcription, and/or overall hydrophobicity of the translated polypeptide. The antigenic peptides can be arranged so that they are joined directly together without linkers, or any combination of linkers between any number of pairs of antigenic peptides, as disclosed in more detail elsewhere herein. The number of linear antigenic peptides to be included can be determined based on consideration of the number of constructs needed versus the mutational burden, the efficiency of translation and secretion of multiple epitopes from a single plasmid, and the MOI needed for each bacteria or Lm comprising a plasmid.

The combination of antigenic peptides or the entire fusion polypeptide (i.e., comprising the antigenic peptides and the PEST-containing peptide and any tags) also be scored for hydrophobicity. For example, the entirety of the fused antigenic peptides or the entire fusion polypeptide can be scored for hydropathy by a Kyte and Doolittle hydropathy index with a sliding 21 amino acid window. If any region scores above a cutoff (e.g., around 1.6), the antigenic peptides can be reordered or shuffled within the fusion polypeptide until an acceptable order of antigenic peptides is found (i.e., one in which no region scores above the cutoff). Alternatively, any problematic antigenic peptides can be removed or redesigned to be of a different size. Alternatively or additionally, one or more linkers between antigenic peptides as disclosed elsewhere herein can be added or modified to change the hydrophobicity. As with hydropathy testing for the individual antigenic peptides, other window sizes can be used, or other cutoffs can be used (e.g., depending on the genus or species of the bacteria being used to deliver the fusion polypeptide). In addition, other suitable hydropathy plots or other appropriate scales could be used.

Optionally, the combination of antigenic peptides or the entire fusion polypeptide can be further screened for immunosuppressive epitopes (e.g., T-reg epitopes, IL-10-inducing T helper epitopes, and so forth) to deselect antigenic peptides or to avoid immunosuppressive influences.

A nucleic acid encoding a candidate combination of antigenic peptides or fusion polypeptide can then be designed and optimized. For example, the sequence can be optimized for increased levels of translation, duration of expression, levels of secretion, levels of transcription, and any combination thereof. For example, the increase can be 2-fold to 1000-fold, 2-fold to 500-fold, 2-fold to 100-fold, 2-fold to 50-fold, 2-fold to 20-fold, 2-fold to 10-fold, or 3-fold to 5-fold relative to a control, non-optimized sequence.

For example, the fusion polypeptide or nucleic acid encoding the fusion polypeptide can be optimized for decreased levels of secondary structures possibly formed in the oligonucleotide sequence, or alternatively optimized to prevent attachment of any enzyme that may modify the sequence. Expression in bacterial cells can be hampered, for example, by transcriptional silencing, low mRNA half-life, secondary structure formation, attachment sites of oligonucleotide binding molecules such as repressors and inhibitors, and availability of rare tRNAs pools. The source of many problems in bacterial expressions is found within the original sequence. The optimization of RNAs may include modification of cis acting elements, adaptation of its GC-content, modifying codon bias with respect to non-limiting tRNAs pools of the bacterial cell, and avoiding internal homologous regions. Thus, optimizing a sequence can entail, for example, adjusting regions of very high (>80%) or very low (<30%) GC content. Optimizing a sequence can also entail, for example, avoiding one or more of the following cis-acting sequence motifs: internal TATA-boxes, chi-sites, and ribosomal entry sites; AT-rich or GC-rich sequence stretches; repeat sequences and RNA secondary structures; (cryptic) splice donor and acceptor sites; branch points; or a combination thereof. Optimizing expression can also entail adding sequence elements to flanking regions of a gene and/or elsewhere in the plasmid.

Optimizing a sequence can also entail, for example, adapting the codon usage to the codon bias of host genes (e.g., Listeria monocytogenes genes). For example, the codons below can be used for Listeria monocytogenes.

```
      A = GCA
      C = TGT
      D = GAT
      E = GAA
      F = TTC
      G = GGT
      H = CAT
      I = ATT
      K = AAA
      L = TTA
      M = ATG
      N = AAC
      P = CCA
      Q = CAA
      R = CGT
      S = TCT
      T = ACA
      V = GTT
      W = TGG
      Y = TAT
STOP = TAA
```

A nucleic acid encoding a fusion polypeptide can be generated and introduced into a delivery vehicle such as a bacteria strain or Listeria strain. Other delivery vehicles may be suitable for DNA immunotherapy or peptide immunotherapy, such as a vaccinia virus or virus-like particle. Once a plasmid encoding a fusion polypeptide is generated and introduced into a bacteria strain or Listeria strain, the bacteria or Listeria strain can be cultured and characterized to confirm expression and secretion of the fusion polypeptide comprising the antigenic peptides.

### III. Recombinant Chimeric Polypeptides Encoded by Minigene Constructs

Disclosed herein are recombinant chimeric polypeptides comprising from N-terminal end to C-terminal end a bacterial secretion signal sequence, a ubiquitin (Ub) protein, and an antigenic WT1 peptide (or one or more antigenic WT1 peptides). If two or more antigenic peptides are included, the antigenic peptides can be in tandem (e.g., Ub-peptide1-peptide2). Alternatively, a combination of separate chimeric polypeptides can be used in which each antigenic WT1 peptide is fused to its own secretion sequence and Ub protein (e.g., Ub1-peptide1; Ub2-peptide2). Examples of suitable antigenic WT1 peptides are disclosed elsewhere herein.

Nucleic acids (termed minigene constructs) encoding such recombinant chimeric polypeptides are also disclosed. Such minigene nucleic acid constructs can further comprise two or more open reading frames linked by a Shine-Dalgarno ribosome binding site nucleic acid sequence between each open reading frame. For example, a minigene nucleic acid construct can further comprise two to four open reading frames linked by a Shine-Dalgarno ribosome binding site nucleic acid sequence between each open reading frame. Each open reading frame can encode a different chimeric polypeptide comprising from N-terminal end to C-terminal end a bacterial secretion sequence, a ubiquitin (Ub) protein, and one or more antigenic WT1 peptides. The codon encoding the carboxy terminus of the chimeric polypeptide can be followed by two stop codons to ensure termination of protein synthesis.

In some chimeric polypeptides encoded by minigene constructs, there are one or more additional antigenic WT1 peptides between the bacterial secretion sequence and the ubiquitin protein. For example, there can be 1-20, 1-15, 1-10, 1-5, 1-4, 1, 2, 3, 4, 5, 6, 7, 8, 9, 10, 11, 12, 13, 14, 15, 16, 17, 18, 18, or 20 additional antigenic WT1 peptides between the bacterial secretion sequence and the ubiquitin protein. If there are two or more additional antigenic WT1 peptides, they can be fused directly to each other are linked via a peptide linker. Exemplary linkers are disclosed elsewhere herein. The additional antigenic WT1 peptides can comprise one or more native WT1 peptides and/or one or more heteroclitic mutant WT1 peptides. Examples of native WT1 peptides include peptides comprising the sequence of any one of SEQ ID NOS: 95, 101-113, 152, 154, 155, 158, and 161. Other examples of suitable native WT1 peptides are disclosed elsewhere herein. Examples of heteroclitic WT1 mutant peptides include peptides comprising the sequence of any one of SEQ ID NOS: 114-137, 159, 160, 162, and 163. Other examples of suitable heteroclitic mutant WT1 peptides are disclosed elsewhere herein. In one example, the one or more additional antigenic peptides comprise, consist essentially of, or consist of a first additional antigenic WT1 peptide comprising, consisting essentially of, or consisting of the sequence set forth in SEQ ID NO: 154, a second additional antigenic WT1 peptide comprising, consisting essentially of, or consisting of the sequence set forth in SEQ ID NO: 155, and a third additional antigenic WT1 peptide comprising, consisting essentially of, or consisting of the sequence set forth in SEQ ID NO: 136. In another example, the one or more additional antigenic peptides comprise, consist essentially of, or consist of a first additional antigenic WT1 peptide comprising, consisting essentially of, or consisting of the sequence set forth in SEQ ID NO: 154, a second additional antigenic WT1 peptide comprising, consisting essentially of, or consisting of the sequence set forth in SEQ ID NO: 155, and a third additional antigenic WT1 peptide comprising, consisting essentially of, or consisting of the sequence set forth in SEQ ID NO: 137. The WT1 antigenic peptide following the ubiquitin in any of these examples can, for example, comprise, consist essentially of, or consist of the sequence set forth in SEQ ID NO; 114 or 115. The resulting chimeric polypeptide can comprise, consist essentially of, or consist of, for example, the sequence set forth in SEQ ID NO: 153.

Examples of bacterial secretion signal sequences are disclosed in more detail elsewhere herein. The ubiquitin can be, for example, a full-length protein. An exemplary ubiquitin peptide encoded by a minigene construct comprises, consists essentially of, or consists of the sequence set forth in SEQ ID NO: 100. The ubiquitin expressed from the nucleic acid construct provided herein can be cleaved at the carboxy terminus of the ubiquitin from the rest of the recombinant fusion polypeptide expressed from the nucleic acid construct through the action of hydrolases upon entry to the host cell cytosol. This liberates the rest of the fusion polypeptide, producing a peptide in the host cell cytosol.

Selection of, variations of, and arrangement of WT1 antigenic peptides within a fusion polypeptide are discussed in detail elsewhere herein, and methods of generating heteroclitic mutant WT1 antigenic peptides are discussed in more detail elsewhere herein.

The recombinant chimeric polypeptides can comprise one or more tags. For example, the recombinant chimeric polypeptides can comprise one or more peptide tags N-terminal and/or C-terminal to the one or more antigenic WT1 peptides. A tag can be fused directly to an antigenic peptide or linked to an antigenic peptide via a linker (examples of which are disclosed elsewhere herein). Examples of tags include the following: FLAG tag; 2×FLAG tag; 3×FLAG tag; His tag, 6×His tag; and SIINFEKL tag. An exemplary SIINFEKL tag is set forth in SEQ ID NO: 16 (encoded by any one of the nucleic acids set forth in SEQ ID NOS: 1-15). An exemplary 3×FLAG tag is set forth in SEQ ID NO: 32 (encoded by any one of the nucleic acids set forth in SEQ ID NOS: 17-31). An exemplary variant 3×FLAG tag is set forth in SEQ ID NO: 99. Two or more tags can be used together, such as a 2×FLAG tag and a SIINFEKL tag, a 3×FLAG tag and a SIINFEKL tag, or a 6×His tag and a SIINFEKL tag. If two or more tags are used, they can be located anywhere within the recombinant fusion polypeptide and in any order. For example, the two tags can be at the C-terminus of the recombinant fusion polypeptide, the two tags can be at the N-terminus of the recombinant fusion polypeptide, the two tags can be located internally within the recombinant fusion polypeptide, one tag can be at the C-terminus and one tag at the N-terminus of the recombinant fusion polypeptide, one tag can be at the C-terminus and one internally within the recombinant fusion polypeptide, or one tag can be at the N-terminus and one internally within the recombinant fusion polypeptide. Other tags include chitin binding protein (CBP), maltose binding protein (MBP), glutathione-S-transferase (GST), thioredoxin (TRX), and poly(NANP). Particular recombinant fusion polypeptides comprise a C-terminal SIINFEKL tag. Such tags can allow for easy detection of the recombinant fusion protein, confirmation of secretion of the recombinant fusion protein, or for following the immunogenicity of the secreted fusion polypeptide by following immune responses to these “tag” sequence peptides. Such immune response can be monitored using a number of reagents including, for example, monoclonal antibodies and DNA or RNA probes specific for these tags.

The recombinant chimeric polypeptides disclosed herein can be expressed by recombinant Listeria strains or can be expressed and isolated from other vectors and cell systems used for protein expression and isolation. Recombinant Listeria strains comprising expressing such antigenic peptides can be used, for example in immunogenic compositions comprising such recombinant Listeria and in vaccines comprising the recombinant Listeria strain and an adjuvant.

Nucleic acids (minigene constructs) encoding such recombinant chimeric polypeptides are also disclosed. The nucleic acid can be in any form. The nucleic acid can comprise or consist of DNA or RNA, and can be single-stranded or double-stranded. The nucleic acid can be in the form of a plasmid, such as an episomal plasmid, a multicopy episomal plasmid, or an integrative plasmid. Alternatively, the nucleic acid can be in the form of a viral vector, a phage vector, or in a bacterial artificial chromosome. Such nucleic acids can have one open reading frame or can have two or more open reading frames (e.g., an open reading frame encoding the recombinant chimeric polypeptide and a second open reading frame encoding a metabolic enzyme). In one example, such nucleic acids can comprise two or more open reading frames linked by a Shine-Dalgarno ribosome binding site nucleic acid sequence between each open reading frame. For example, a nucleic acid can comprise two to four open reading frames linked by a Shine-Dalgarno ribosome binding site nucleic acid sequence between each open reading frame. Each open reading frame can encode a different polypeptide. The codon encoding the carboxy terminus of the chimeric polypeptide can be followed by two stop codons to ensure termination of protein synthesis.

Some exemplary recombinant chimeric polypeptides includes those with sequences comprising, consisting essentially of, or consisting of the sequence set forth in any one of SEQ ID NOS: 153, 156, 157, and 164-166.

A. Antigenic WT1 Peptides Encoded by Minigene Constructs

WT1 peptides encoded by the minigene constructs disclosed herein can be native (i.e., unaltered, naturally occurring) WT1 peptides or heteroclitic WT1 peptides (e.g., HLA class I and class II heteroclitic peptides). Examples of native WT1 peptides include peptides comprising the sequence of any one of SEQ ID NOS: 95, 101-113, 152, 154, 155, 158, and 161. Examples of heteroclitic WT1 peptides include peptides comprising the sequence of any one of SEQ ID NOS: 114-137, 159, 160, 162, and 163. The term “heteroclitic” refers to a peptide that generates an immune response that recognizes the native peptide from which the heteroclitic peptide was derived (e.g., the peptide not containing the anchor residue mutations). For example, FMFPNAPYL (SEQ ID NO: 114) was generated from RMFPNAPYL (SEQ ID NO: 101) by mutation of residue 1 to phenylalanine. A heteroclitic peptide can generate an immune response that recognizes the native peptide from which the heteroclitic peptide was derived. For example, the immune response against the native peptide generated by vaccination with the heteroclitic peptide can be equal or greater in magnitude than the immune response generated by vaccination with the native peptide. The immune response can be increased, for example, by 2-fold, 3-fold, 5-fold, 7-fold, 10-fold, 15-fold, 20-fold, 30-fold, 50-fold, 100-fold, 150-fold, 200-fold, 300-fold, 500-fold, 1000-fold, or more.

A native or heteroclitic WT1 peptide disclosed herein can bind to one or more HLA molecules. HLA molecules, also known as major histocompatibility complex (MHC) molecules, bind peptides and present them to immune cells. The immunogenicity of a peptide can be partially determined by its affinity for HLA molecules. HLA class I molecules interact with CD8 molecules, which are generally present on cytotoxic T lymphocytes (CTL). HLA class II molecules interact with CD4 molecules, which are generally present on helper T lymphocytes. For example, a WT1 peptide disclosed herein can bind to an HLA molecule with sufficient affinity to activate a T cell precursor or with sufficient affinity to mediate recognition by a T cell.

A native or heteroclitic WT1 peptide disclosed herein can bind to one or more HLA class II molecules. For example, a WT1 peptide can bind to an HLA-DRB molecule, an HLA-DRA molecule, an HLA-DQA1 molecule, an HLA-DQB1 molecule, an HLA-DPA1 molecule, an HLA-DPB 1 molecule, an HLA-DMA molecule, an HLA-DMB molecule, an HLA-DOA molecule, or an HLA-DOB molecule.

A native or heteroclitic WT1 peptide disclosed herein can bind to one or more HLA class I molecules. For example, a WT1 peptide can bind to an HLA-A molecule, an HLA-B molecule, an HLA-C molecule, an HLA-A0201 molecule, HLA A1, HLA A2, HLA A2.1, HLA A3, HLA A3.2, HLA A11, HLA A24, HLA B7, HLA B27, or HLA B8. Similarly, a WT-1 peptide can bind to a superfamily of HLA class I molecules, such as the A2 superfamily, the A3 superfamily, the A24 superfamily, the B7 superfamily, the B27 superfamily, the B44 superfamily, the C1 superfamily, or the C4 superfamily.

Heteroclitic WT1 peptides can comprise a mutation that enhances binding of the peptide to an HLA class II molecule relative to the corresponding native WT1 peptide. Alternatively, or additionally, heteroclitic WT1 peptides can comprise a mutation that enhances binding of the peptide to an HLA class I molecule relative to the corresponding native WT1 peptide. For example, the mutated residue can be an HLA class II motif anchor residue. “Anchor motifs” or “anchor residues” refers, in another embodiment, to one or a set of preferred residues at particular positions in an HLA-binding sequence (e.g., an HLA class II binding sequence or an HLA class I binding sequence).

Various methods are well-known for generating predicted heteroclitic epitopes with the potential to elicit cross-reactive immunogenic responses to a wild-type epitope. For example, to design heteroclitic epitopes with the potential to elicit cross-reactive immunogenic responses to a wild-type epitope, baseline predicted peptide-MHC binding affinity of the wild-type epitopes can be determined using NetMHCpan 3.0 Server (www.cbs.dtu.dk/services/NetMHCpan/). A peptide-MHC binding affinity percent rank of less than or equal to 1.0 is considered a strong binder that is likely to elicit an immune response. Potential heteroclitic epitopes are generated by random substitution of 1 or more amino acids at, but not limited to, positions 1, 2, 3, or the C-terminal position of the wild-type epitope that is predicted to be a strong binder. The peptide-MHC binding affinity of the potential heteroclitic epitopes is then estimated using NetMHCpan 3.0 Server. Heteroclitic epitopes with percentage ranking binding affinities similar to wild-type epitopes and less than or equal to 1.0 percentage rank can be considered potential antigens for future validation.

Other methods for identifying HLA class I and class II residues, and for improving HLA binding by mutating the residues, are well-known. See, e.g., U.S. Pat. Nos. 8,765,687, 7,488,718, 9,233,149, and 7,598,221, each of which is herein incorporated by reference in its entirety for all purposes. For example, methods for predicting MHC class II epitopes are well-known. As one example, the MHC class II epitope can be predicted using TEPITOPE (Meister et al. (1995) Vaccine 13:581-591, herein incorporated by reference in its entirety for all purposes). As another example, the MHC class II epitope can be predicted using EpiMatrix (De Groot et al. (1997) AIDS Res. Hum. Retroviruses 13:529-531, herein incorporated by reference in its entirety for all purposes). As yet another example, the MHC class II epitope can be predicted using the Predict Method (Yu K et al. (2002) Mol. Med. 8:137-148, herein incorporated by reference in its entirety for all purposes). As yet another example, the MHC class II epitope can be predicted using the SYFPEITHI epitope prediction algorithm. SYFPEITHI is a database comprising more than 4500 peptide sequences known to bind class I and class II MHC molecules. SYFPEITHI provides a score based on the presence of certain amino acids in certain positions along the MHC-binding groove. Ideal amino acid anchors are valued at 10 points, unusual anchors are worth 6-8 points, auxiliary anchors are worth 4-6 points, preferred residues are worth 1-4 points; negative amino acid effect on the binding score between −1 and −3. The maximum score for HLA-A*0201 is 36. As yet another example, the MHC class II epitope can be predicted using Rankpep. Rankpep uses position specific scoring matrices (PSSMs) or profiles from sets of aligned peptides known to bind to a given MHC molecule as the predictor of MHC-peptide binding. Rankpep includes information on the score of the peptide and the % optimum or percentile score of the predicted peptide relative to that of a consensus sequence that yields the maximum score, with the selected profile. Rankpep includes a selection of 102 and 80 PSSMs for the prediction of peptide binding to MHC I and MHC II molecules, respectively. Several PSSMs for the prediction of peptide binders of different sizes are usually available for each MHC I molecule. As another example, the MHC class II epitope can be identified using SVMHC (Donnes and Elofsson (2002) BMC Bioinformatics 11; 3:25, herein incorporated by reference in its entirety for all purposes).

Methods for identifying MHC class I epitopes are also well-known. As one example, the MHC class I epitope can be predicted using BIMAS software. A BIMAS score is based on the calculation of the theoretical half-life of the MHC-I/β₂-microglobulin/peptide complex, which is a measure of peptide-binding affinity. The program uses information about HLA-I peptides of 8-10 amino acids in length. The higher the binding affinity of a peptide to the MHC, the higher the likelihood that this peptide represents an epitope. The BIMAS algorithm assumes that each amino acid in the peptide contributes independently to binding to the class I molecule. Dominant anchor residues, which are critical for binding, have coefficients in the tables that are significantly higher than 1. Unfavorable amino acids have positive coefficients that are less than 1. If an amino acid is not known to make either a favorable or unfavorable contribution to binding, then it is assigned the value 1. All the values assigned to the amino acids are multiplied and the resulting running score is multiplied by a constant to yield an estimate of half-time of dissociation. As another example, the MHC class I epitope can be identified using SYFPEITHI. As yet another example, the MHC class I epitope can be identified using SVMHC. As yet another example, the MHC class I epitope can be identified using NetMHC-2.0 (Buus et al. (2003) Tissue Antigens 62:378-384, herein incorporated by reference in its entirety for all purposes).

Different residues in HLA binding motifs can be mutated to enhance MHC binding. In one example, a mutation that enhances MHC binding is in the residue at position 1 of the HLA class I binding motif (e.g., a mutation to tyrosine, glycine, threonine, or phenylalanine). As another example, the mutation can be in position 2 of the HLA class I binding motif (e.g., a mutation to leucine, valine, isoleucine, or methionine). As another example, the mutation can be in position 6 of the HLA class I binding motif (e.g., to valine, cysteine, glutamine, or histidine). As another example, the mutation can be in position 9 of the HLA class I binding motif or in the C-terminal position (e.g., to valine, threonine, isoleucine, leucine, alanine, or cysteine). The mutation can be in a primary anchor residue or in a secondary anchor residue. For example, the HLA class I primary anchor residues can be positions 2 and 9, and the secondary anchor residues can be positions 1 and 8 or positions 1, 3, 6, 7, and 8. In another example, a point mutation can be in a position selected from positions 4, 5, and 8.

Similarly, different residues in HLA class II binding sites can be mutated. For example, an HLA class II motif anchor residue can be modified. For example, the P1 position, the P2 position, the P6 position, or the P9 position can be mutated. Alternatively, the P4 position, the P5 position, the P10 position, the P11 position, the P12 position, or the P13 position can be mutated.

The chimeric polypeptide encoded by the minigene construct can include a single antigenic WT1 peptide or can include two or more antigenic peptides. Each antigenic peptide can be of any length sufficient to induce an immune response, and each antigenic peptide can be the same length or the antigenic peptides can have different lengths. For example, an antigenic peptide encoded by a minigene construct can be 8-100, 8-50, 8-30, 8-25, 8-22, 8-20, 8-15, 8-14, 8-13, 8-12, 8-11, or 8-10 amino acids in length. In one example, an antigenic WT1 peptide can be 9 amino acids in length.

Each antigenic peptide can also be hydrophilic or can score up to or below a certain hydropathy threshold, which can be predictive of secretability in Listeria monocytogenes or another bacteria of interest. For example, antigenic peptides can be scored by a Kyte and Doolittle hydropathy index 21 amino acid window, and all scoring above a cutoff (around 1.6) can be excluded as they are unlikely to be secretable by Listeria monocytogenes. Likewise, the combination of antigenic peptides or the chimeric polypeptide can be hydrophilic or can score up to or below a certain hydropathy threshold, which can be predictive of secretability in Listeria monocytogenes or another bacteria of interest.

If the chimeric polypeptide includes more than one antigenic peptide, the antigenic peptides can be linked together in any manner. For example, the antigenic peptides can be fused directly to each other with no intervening sequence. Alternatively, the antigenic peptides can be linked to each other indirectly via one or more linkers, such as peptide linkers. In some cases, some pairs of adjacent antigenic peptides can be fused directly to each other, and other pairs of antigenic peptides can be linked to each other indirectly via one or more linkers. The same linker can be used between each pair of adjacent antigenic peptides, or any number of different linkers can be used between different pairs of adjacent antigenic peptides. In addition, one linker can be used between a pair of adjacent antigenic peptides, or multiple linkers can be used between a pair of adjacent antigenic peptides.

Any suitable sequence can be used for a peptide linker. Examples of suitable linkers are disclosed elsewhere herein.

Exemplary native and corresponding heteroclitic mutant WT1 peptides encoded by minigene constructs are provided in the following table. Mutated residues in the heteroclitic peptides are bolded and underlined. Other exemplary native WT1 peptides include SEQ ID NOS: 95 and 152.

| Type of WT1 | | SEQ | BIMAS |
|---|---|---|---|
| Peptide | Sequence | ID NO | Score |
| Native | RMFPNAPYL | 101 | 313 |
| Heteroclitic | YMFPNAPYL | 115 | 1444 |
| Native | SLGEQQYSV | 102 | 285 |
| Heteroclitic | YLGEQQYSV | 116 | 1311 |
| Native | ALLPAVPSL | 103 | 181 |
| Heteroclitic | YLLPAVPSL | 117 | 836 |
| Native | NLGATLKGV | 104 | 159 |
| Heteroclitic | YLGATLKGV | 118 | 735 |
| Native | DLNALLPAV | 105 | 11 |
| Heteroclitic | YLNALLPAV | 119 | 735 |
| Native | GVFRGIQDV | 106 | 51 |
| Heteroclitic | GLRRGIQDV | 120 | 591 |
| Native | KRYFKLSHL | 107 | 1 |
| Heteroclitic | KLYFKLSHL | 121 | 550 |
| Native | ALLLRTPYS | 108 | 1 |
| Heteroclitic | ALLLRTPYV | 122 | 1415 |
| Native | CMTWNQMNL | 109 | 15 |
| Heteroclitic | YMTWNQMNL | 123 | 70 |
| Native | NMHQRNMTK | 110 | 40 |
| Heteroclitic | NMYQRNMTK | 124 | 200 |
| Heteroclitic | NMHQRVMTK | 125 | 120 |
| Heteroclitic | NMYQRVMTK | 126 | 600 |
| Native | QMNLGATLK | 111 | 20 |
| Heteroclitic | QMYLGATLK | 127 | 100 |
| Heteroclitic | QMNLGVTLK | 128 | 60 |
| Heteroclitic | QMYLGVTLK | 129 | 300 |
| Native | FMCAYPGCNK | 112 | 30 |
| Heteroclitic | FMYAYPGCNK | 130 | 150 |
| Heteroclitic | FMCAYPFCNK | 131 | 90 |
| Heteroclitic | FMYAYPFCNK | 132 | 450 |
| Native | KLSHLQMHSR | 113 | 18 |
| Heteroclitic | KLYHLQMHSR | 133 | 90 |
| Heteroclitic | KLSHLQMHSK | 134 | 90 |
| Heteroclitic | KLYHLQMHSK | 135 | 450 |

Other exemplary native WT1 peptides and corresponding heteroclitic mutant WT1 peptides encoded by minigene constructs are set forth in the table below. Mutated residues in the heteroclitic peptides are bolded and underlined.

| Type of WT1 | | SEQ |
|---|---|---|
| Peptide | Sequence | ID NO |
| Native | SGQARMFPNAPYLPSCLES | 152 |
| (WT1-122A long) | | |
| Heteroclitic | SGQAYMFPNAPYLPSCLES | 137 |
| (WT1-122A1 long) | | |
| Native | NQMNLGATL | 158 |
| (A24-native) | | |
| Heteroclitic | NLMNLGATL | 159 |
| (A24-het-1) | | |
| Heteroclitic | NYMNLGATL | 160 |
| (A24-het-2) | | |
| Native | WNQMNLGATLKGVAA | 161 |
| (A24-native-long) | | |
| Heteroclitic | WNLMNLGATLKGVAA | 162 |
| (A24-het-1-long) | | |
| Heteroclitic | WNYMNLGATLKGVAA | 163 |
| (A24-het-2-long) | | |
| Native | RSDELVRHHNMHQRNMTKL | 154 |
| (WT1-427 long) | | |
| Native | PGCNKRYFKLSHLQMHSRKHTG | 155 |
| (WT1-331 long) | | |

B. Bacterial Secretion Signal Sequences

The bacterial secretion signal sequence can be a Listerial signal sequence, such as an Hly or an ActA signal sequence, or any other known signal sequence. In other cases, the signal sequence can be an LLO signal sequence. An exemplary LLO signal sequence is set forth in SEQ ID NO: 150. For example, a bacterial secretion signal sequence encoded by a minigene construct herein can be an N-terminal fragment of LLO such as that set forth in SEQ ID NO: 59. The signal sequence can be bacterial, can be native to a host bacterium (e.g., Listeria monocytogenes, such as a secA1 signal peptide), or can be foreign to a host bacterium. Specific examples of signal peptides include an Usp45 signal peptide from Lactococcus lactis, a Protective Antigen signal peptide from Bacillus anthracis, a secA2 signal peptide such the p60 signal peptide from Listeria monocytogenes, and a Tat signal peptide such as a B. subtilis Tat signal peptide (e.g., PhoD). In specific examples, the secretion signal sequence is from a Listeria protein, such as an ActA₃₀₀ secretion signal or an ActA₁₀₀ secretion signal (comprising the first 100 amino acids of the ActA secretion signal sequence). An exemplary ActA signal sequence is set forth in SEQ ID NO: 151.

C. Generating Immunotherapy Constructs Encoding Recombinant Chimeric Polypeptides Encoded by Minigene Constructs

Also provided herein are methods for generating immunotherapy constructs encoding or compositions comprising the recombinant chimeric polypeptides disclosed herein. For example, such methods can comprise selecting and designing antigenic or immunogenic peptides to include in the immunotherapy construct (and, for example, testing the hydropathy of the each antigenic peptide, and modifying or deselecting an antigenic peptide if it scores above a selected hydropathy index threshold value), designing one or more fusion polypeptides comprising each of the selected antigenic peptides, and generating a nucleic acid construct encoding the fusion polypeptide. Such methods are disclosed in more detail elsewhere herein. In addition, methods for generating predicted heteroclitic epitopes with the potential to elicit cross-reactive immunogenic responses to a wild-type epitope are described in more detail elsewhere herein.

### IV. Recombinant Bacteria or Listeria Strains

Also provided herein are recombinant bacterial strains, such as a Listeria strain, comprising a recombinant fusion polypeptide or chimeric polypeptide disclosed herein or a nucleic acid encoding the recombinant fusion polypeptide or chimeric polypeptide (e.g., minigene construct) as disclosed elsewhere herein. Preferably, the bacterial strain is a Listeria strain, such as a Listeria monocytogenes (Lm) strain. Lm has a number of inherent advantages as a vaccine vector. The bacterium grows very efficiently in vitro without special requirements, and it lacks LPS, which is a major toxicity factor in gram-negative bacteria, such as Salmonella. Genetically attenuated Lm vectors also offer additional safety as they can be readily eliminated with antibiotics, in case of serious adverse effects, and unlike some viral vectors, no integration of genetic material into the host genome occurs.

The recombinant Listeria strain can be any Listeria strain. Examples of suitable Listeria strains include Listeria seeligeri, Listeria grayi, Listeria ivanovii, Listeria murrayi, Listeria welshimeri, Listeria monocytogenes (Lm), or any other Listeria species known in the art. Preferably, the recombinant listeria strain is a strain of the species Listeria monocytogenes. Examples of Listeria monocytogenes strains include the following: L. monocytogenes 10403S wild type (see, e.g., Bishop and Hinrichs (1987) J Immunol 139:2005-2009; Lauer et al. (2002) J Bact 184:4177-4186); L. monocytogenes DP-L4056, which is phage cured (see, e.g., Lauer et al. (2002) J Bact 184:4177-4186); L. monocytogenes DP-L4027, which is phage cured and has an hly gene deletion (see, e.g., Lauer et al. (2002) J Bact 184:4177-4186; Jones and Portnoy (1994) Infect Immunity 65:5608-5613); L. monocytogenes DP-L4029, which is phage cured and has an actA gene deletion (see, e.g., Lauer et al. (2002) J Bact 184:4177-4186; Skoble et al. (2000) J Cell Biol 150:527-538); L. monocytogenes DP-L4042 (delta PEST) (see, e.g., Brockstedt et al. (2004) Proc Natl Acad Sci. USA 101:13832-13837 and supporting information); L. monocytogenes DP-L4097 (LLO-S44A) (see, e.g., Brockstedt et al. (2004) Proc Natl Acad Sci USA 101:13832-13837 and supporting information); L. monocytogenes DP-L4364 (delta lplA; lipoate protein ligase) (see, e.g., Brockstedt et al. (2004) Proc Natl Acad Sci USA 101:13832-13837 and supporting information); L. monocytogenes DP-L4405 (delta inlA) (see, e.g., Brockstedt et al. (2004) Proc Natl Acad Sci USA 101:13832-13837 and supporting information); L. monocytogenes DP-L4406 (delta inlB) (see, e.g., Brockstedt et al. (2004) Proc Natl Acad Sci USA 101:13832-13837 and supporting information); L. monocytogenes CS-LOOO1 (delta actA; delta inlB) (see, e.g., Brockstedt et al. (2004) Proc Natl Acad Sci USA 101:13832-13837 and supporting information); L. monocytogenes CS-L0002 (delta actA; delta lplA) (see, e.g., Brockstedt et al. (2004) Proc Natl Acad Sci USA 101:13832-13837 and supporting information); L. monocytogenes CS-L0003 (LLO L461T; delta lplA) (see, e.g., Brockstedt et al. (2004) Proc Natl Acad Sci USA 101:13832-13837 and supporting information); L. monocytogenes DP-L4038 (delta actA; LLO L461T) (see, e.g., Brockstedt et al. (2004) Proc Natl Acad Sci USA 101:13832-13837 and supporting information); L. monocytogenes DP-L4384 (LLO S44A; LLO L461T) (see, e.g., Brockstedt et al. (2004) Proc Natl Acad Sci USA 101:13832-13837 and supporting information); a L. monocytogenes strain with an lplA1 deletion (encoding lipoate protein ligase LplA1) (see, e.g., O'Riordan et al. (2003) Science 302:462-464); L. monocytogenes DP-L4017 (10403S with LLO L461T) (see, e.g., U.S. Pat. No. 7,691,393); L. monocytogenes EGD (see, e.g., GenBank Accession No. AL591824). In another embodiment, the Listeria strain is L. monocytogenes EGD-e (see GenBank Accession No. NC_003210; ATCC Accession No. BAA-679); L. monocytogenes DP-L4029 (actA deletion, optionally in combination with uvrAB deletion (DP-L4029uvrAB) (see, e.g., U.S. Pat. No. 7,691,393); L. monocytogenes actA-linlB—double mutant (see, e.g., ATCC Accession No. PTA-5562); L. monocytogenes lplA mutant or hly mutant (see, e.g., US 2004/0013690); L. monocytogenes dal/dat double mutant (see, e.g., US 2005/0048081). Other L. monocytogenes strains includes those that are modified (e.g., by a plasmid and/or by genomic integration) to contain a nucleic acid encoding one of, or any combination of, the following genes: hly (LLO; listeriolysin); iap (p60); inlA; inlB; inlC; dal (alanine racemase); dat (D-amino acid aminotransferase); plcA; plcB; actA; or any nucleic acid that mediates growth, spread, breakdown of a single walled vesicle, breakdown of a double walled vesicle, binding to a host cell, or uptake by a host cell. Each of the above references is herein incorporated by reference in its entirety for all purposes.

The recombinant bacteria or Listeria can have wild-type virulence, can have attenuated virulence, or can be avirulent. For example, a recombinant Listeria of can be sufficiently virulent to escape the phagosome or phagolysosome and enter the cytosol. Such Listeria strains can also be live-attenuated Listeria strains, which comprise at least one attenuating mutation, deletion, or inactivation as disclosed elsewhere herein. Preferably, the recombinant Listeria is an attenuated auxotrophic strain. An auxotrophic strain is one that is unable to synthesize a particular organic compound required for its growth. Examples of such strains are described in U.S. Pat. No. 8,114,414, herein incorporated by reference in its entirety for all purposes.

Preferably, the recombinant Listeria strain lacks antibiotic resistance genes. For example, such recombinant Listeria strains can comprise a plasmid that does not encode an antibiotic resistance gene. However, some recombinant Listeria strains provided herein comprise a plasmid comprising a nucleic acid encoding an antibiotic resistance gene. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin.

A. Bacteria or Listeria Strains Comprising Recombinant Fusion Polypeptides or Chimeric Polypeptides or Nucleic Acids Encoding Recombinant Fusion Polypeptides or Chimeric Polypeptides

The recombinant bacterial strains (e.g., Listeria strains) disclosed herein comprise a recombinant fusion polypeptide or chimeric polypeptide disclosed herein or a nucleic acid encoding the recombinant fusion polypeptide or chimeric polypeptide (e.g., minigene construct) as disclosed elsewhere herein.

In bacteria or Listeria strains comprising a nucleic acid encoding a recombinant fusion protein, the nucleic acid can be codon optimized. Examples of optimal codons utilized by L. monocytogenes for each amino acid are shown US 2007/0207170, herein incorporated by reference in its entirety for all purposes. A nucleic acid is codon-optimized if at least one codon in the nucleic acid is replaced with a codon that is more frequently used by L. monocytogenes for that amino acid than the codon in the original sequence.

The nucleic acid can be present in an episomal plasmid within the bacteria or Listeria strain and/or the nucleic acid can be genomically integrated in the bacteria or Listeria strain. Some recombinant bacteria or Listeria strains comprise two separate nucleic acids encoding two recombinant fusion polypeptides or chimeric polypeptides as disclosed herein: one nucleic acid in an episomal plasmid, and one genomically integrated in the bacteria or Listeria strain.

The episomal plasmid can be one that is stably maintained in vitro (in cell culture), in vivo (in a host), or both in vitro and in vivo. If in an episomal plasmid, the open reading frame encoding the recombinant fusion polypeptide or chimeric polypeptide can be operably linked to a promoter/regulatory sequence in the plasmid. If genomically integrated in the bacteria or Listeria strain, the open reading frame encoding the recombinant fusion polypeptide or chimeric polypeptide can be operably linked to an exogenous promoter/regulatory sequence or to an endogenous promoter/regulatory sequence. Examples of promoters/regulatory sequences useful for driving constitutive expression of a gene are well known and include, for example, an hly, hlyA, actA, prfA, and p60 promoters of Listeria, the Streptococcus bac promoter, the Streptomyces griseus sgiA promoter, and the B. thuringiensis phaZ promoter. In some cases, an inserted gene of interest is not interrupted or subjected to regulatory constraints which often occur from integration into genomic DNA, and in some cases, the presence of the inserted heterologous gene does not lead to rearrangement or interruption of the cell's own important regions.

Such recombinant bacteria or Listeria strains can be made by transforming a bacteria or Listeria strain or an attenuated bacteria or Listeria strain described elsewhere herein with a plasmid or vector comprising a nucleic acid encoding the recombinant fusion polypeptide or chimeric polypeptide. The plasmid can be an episomal plasmid that does not integrate into a host chromosome. Alternatively, the plasmid can be an integrative plasmid that integrates into a chromosome of the bacteria or Listeria strain. The plasmids used herein can also be multicopy plasmids. Methods for transforming bacteria are well known, and include calcium-chloride competent cell-based methods, electroporation methods, bacteriophage-mediated transduction, chemical transformation techniques, and physical transformation techniques. See, e.g., de Boer et al. (1989) Cell 56:641-649; Miller et al. (1995) FASEB J. 9:190-199; Sambrook et al. (1989) Molecular Cloning: A Laboratory Manual, Cold Spring Harbor Laboratory, New York; Ausubel et al. (1997) Current Protocols in Molecular Biology, John Wiley & Sons, New York; Gerhardt et al., eds., 1994, Methods for General and Molecular Bacteriology, American Society for Microbiology, Washington, D.C.; and Miller, 1992, A Short Course in Bacterial Genetics, Cold Spring Harbor Laboratory Press, Cold Spring Harbor, N.Y., each of which is herein incorporated by reference in its entirety for all purposes.

Bacteria or Listeria strains with genomically integrated heterologous nucleic acids can be made, for example, by using a site-specific integration vector, whereby the bacteria or Listeria comprising the integrated gene is created using homologous recombination. The integration vector can be any site-specific integration vector that is capable of infecting a bacteria or Listeria strain. Such an integration vector can comprise, for example, a PSA attPP′ site, a gene encoding a PSA integrase, a U153 attPP′ site, a gene encoding a U153 integrase, an A118 attPP′ site, a gene encoding an A118 integrase, or any other known attPP′ site or any other phage integrase.

Such bacteria or Listeria strains comprising an integrated gene can also be created using any other known method for integrating a heterologous nucleic acid into a bacteria or Listeria chromosome. Techniques for homologous recombination are well known, and are described, for example, in Baloglu et al. (2005) Vet Microbial 109(1-2):11-17); Jiang et al. 2005) Acta Biochim Biophys Sin (Shanghai) 37(1):19-24), and U.S. Pat. No. 6,855,320, each of which is herein incorporated by reference in its entirety for all purposes.

Integration into a bacteria or Listerial chromosome can also be achieved using transposon insertion. Techniques for transposon insertion are well known, and are described, for example, for the construction of DP-L967 by Sun et al. (1990) Infection and Immunity 58: 3770-3778, herein incorporated by reference in its entirety for all purposes. Transposon mutagenesis can achieve stable genomic insertion, but the position in the genome where the heterologous nucleic acids has been inserted is unknown.

Integration into a bacterial or Listerial chromosome can also be achieved using phage integration sites (see, e.g., Lauer et al. (2002) J Bacteriol 184(15):4177-4186, herein incorporated by reference in its entirety for all purposes). For example, an integrase gene and attachment site of a bacteriophage (e.g., U153 or PSA listeriophage) can be used to insert a heterologous gene into the corresponding attachment site, which may be any appropriate site in the genome (e.g. comK or the 3′ end of the arg tRNA gene). Endogenous prophages can be cured from the utilized attachment site prior to integration of the heterologous nucleic acid. Such methods can result, for example, in single-copy integrants. In order to avoid a “phage curing step,” a phage integration system based on PSA phage can be used (see, e.g., Lauer et al. (2002) J Bacteriol 184:4177-4186, herein incorporated by reference in its entirety for all purposes). Maintaining the integrated gene can require, for example, continuous selection by antibiotics. Alternatively, a phage-based chromosomal integration system can be established that does not require selection with antibiotics. Instead, an auxotrophic host strain can be complemented. For example, a phage-based chromosomal integration system for clinical applications can be used, where a host strain that is auxotrophic for essential enzymes, including, for example, D-alanine racemase is used (e.g., Lm dal(−)dat(−)).

Conjugation can also be used to introduce genetic material and/or plasmids into bacteria. Methods for conjugation are well known, and are described, for example, in Nikodinovic et al. (2006) Plasmid 56(3):223-227 and Auchtung et al. (2005) Proc Natl Acad Sci USA 102(35):12554-12559, each of which is herein incorporated by reference in its entirety for all purposes.

In a specific example, a recombinant bacteria or Listeria strain can comprise a nucleic acid encoding a recombinant fusion polypeptide or chimeric polypeptide genomically integrated into the bacteria or Listeria genome as an open reading frame with an endogenous actA sequence (encoding an ActA protein) or an endogenous hly sequence (encoding an LLO protein). For example, the expression and secretion of the fusion polypeptide or chimeric polypeptide can be under the control of the endogenous actA promoter and ActA signal sequence or can be under the control of the endogenous hly promoter and LLO signal sequence. As another example, the nucleic acid encoding a recombinant fusion polypeptide or chimeric polypeptide can replace an actA sequence encoding an ActA protein or an hly sequence encoding an LLO protein.

Selection of recombinant bacteria or Listeria strains can be achieved by any means. For example, antibiotic selection can be used. Antibiotic resistance genes may be used in the conventional selection and cloning processes commonly employed in molecular biology and vaccine preparation. Exemplary antibiotic resistance genes include gene products that confer resistance to ampicillin, penicillin, methicillin, streptomycin, erythromycin, kanamycin, tetracycline, chloramphenicol (CAT), neomycin, hygromycin, and gentamicin. Alternatively, auxotrophic strains can be used, and an exogenous metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. Alternatively, a temperature-sensitive plasmid can be used to select recombinants or any other known means for selecting recombinants.

B. Attenuation of Bacteria or Listeria Strains

The recombinant bacteria strains (e.g., recombinant Listeria strains) disclosed herein can be attenuated. The term “attenuation” encompasses a diminution in the ability of the bacterium to cause disease in a host animal. For example, the pathogenic characteristics of an attenuated Listeria strain may be lessened compared with wild-type Listeria, although the attenuated Listeria is capable of growth and maintenance in culture. Using as an example the intravenous inoculation of BALB/c mice with an attenuated Listeria, the lethal dose at which 50% of inoculated animals survive (LD₅₀) is preferably increased above the LD₅₀ of wild-type Listeria by at least about 10-fold, more preferably by at least about 100-fold, more preferably at least about 1,000 fold, even more preferably at least about 10,000 fold, and most preferably at least about 100,000-fold. An attenuated strain of Listeria is thus one that does not kill an animal to which it is administered, or is one that kills the animal only when the number of bacteria administered is vastly greater than the number of wild-type non-attenuated bacteria which would be required to kill the same animal. An attenuated bacterium should also be construed to mean one which is incapable of replication in the general environment because the nutrient required for its growth is not present therein. Thus, the bacterium is limited to replication in a controlled environment wherein the required nutrient is provided. Attenuated strains are environmentally safe in that they are incapable of uncontrolled replication

(I) Methods of Attenuating Bacteria and Listeria Strains

Attenuation can be accomplished by any known means. For example, such attenuated strains can be deficient in one or more endogenous virulence genes or one or more endogenous metabolic genes. Examples of such genes are disclosed herein, and attenuation can be achieved by inactivation of any one of or any combination of the genes disclosed herein. Inactivation can be achieved, for example, through deletion or through mutation (e.g., an inactivating mutation). The term “mutation” includes any type of mutation or modification to the sequence (nucleic acid or amino acid sequence) and may encompass a deletion, a truncation, an insertion, a substitution, a disruption, or a translocation. For example, a mutation can include a frameshift mutation, a mutation which causes premature termination of a protein, or a mutation of regulatory sequences which affect gene expression. Mutagenesis can be accomplished using recombinant DNA techniques or using traditional mutagenesis technology using mutagenic chemicals or radiation and subsequent selection of mutants. Deletion mutants may be preferred because of the accompanying low probability of reversion. The term “metabolic gene” refers to a gene encoding an enzyme involved in or required for synthesis of a nutrient utilized or required by a host bacteria. For example, the enzyme can be involved in or required for the synthesis of a nutrient required for sustained growth of the host bacteria. The term “virulence” gene includes a gene whose presence or activity in an organism's genome that contributes to the pathogenicity of the organism (e.g., enabling the organism to achieve colonization of a niche in the host (including attachment to cells), immunoevasion (evasion of host's immune response), immunosuppression (inhibition of host's immune response), entry into and exit out of cells, or obtaining nutrition from the host).

A specific example of such an attenuated strain is Listeria monocytogenes (Lm) dal(−) dat(−) (Lmdd). Another example of such an attenuated strain is Lm dal(−)dat(−)ΔactA (LmddA). See, e.g., US 2011/0142791, herein incorporated by references in its entirety for all purposes. LmddA is based on a Listeria strain which is attenuated due to the deletion of the endogenous virulence gene actA. Such strains can retain a plasmid for antigen expression in vivo and in vitro by complementation of the dal gene. Alternatively, the LmddA can be a dal/dat/actA Listeria having mutations in the endogenous dal, dat, and actA genes. Such mutations can be, for example, a deletion or other inactivating mutation.

Another specific example of an attenuated strain is Lm prfA(−) or a strain having a partial deletion or inactivating mutation in the prfA gene. The PrfA protein controls the expression of a regulon comprising essential virulence genes required by Lm to colonize its vertebrate hosts; hence the prfA mutation strongly impairs PrfA ability to activate expression of PrfA-dependent virulence genes.

Yet another specific example of an attenuated strain is Lm inlB(−)actA(−) in which two genes critical to the bacterium's natural virulence—internalin B and act A—are deleted.

Other examples of attenuated bacteria or Listeria strains include bacteria or Listeria strains deficient in one or more endogenous virulence genes. Examples of such genes include actA, prfA, plcB, plcA, inlA, inlB, inlC, inlJ, and bsh in Listeria. Attenuated Listeria strains can also be the double mutant or triple mutant of any of the above-mentioned strains. Attenuated Listeria strains can comprise a mutation or deletion of each one of the genes, or comprise a mutation or deletion of, for example, up to ten of any of the genes provided herein (e.g., including the actA, prfA, and dal/dat genes). For example, an attenuated Listeria strain can comprise a mutation or deletion of an endogenous internalin C (inlC) gene and/or a mutation or deletion of an endogenous actA gene. Alternatively, an attenuated Listeria strain can comprise a mutation or deletion of an endogenous internalin B (inlB) gene and/or a mutation or deletion of an endogenous actA gene. Alternatively, an attenuated Listeria strain can comprise a mutation or deletion of endogenous inlB, inlC, and actA genes. Translocation of Listeria to adjacent cells is inhibited by the deletion of the endogenous actA gene and/or the endogenous inlC gene or endogenous inlB gene, which are involved in the process, thereby resulting in high levels of attenuation with increased immunogenicity and utility as a strain backbone. An attenuated Listeria strain can also be a double mutant comprising mutations or deletions of both plcA and plcB. In some cases, the strain can be constructed from the EGD Listeria backbone.

A bacteria or Listeria strain can also be an auxotrophic strain having a mutation in a metabolic gene. As one example, the strain can be deficient in one or more endogenous amino acid metabolism genes. For example, the generation of auxotrophic strains of Listeria deficient in D-alanine, for example, may be accomplished in a number of ways that are well known, including deletion mutations, insertion mutations, frameshift mutations, mutations which cause premature termination of a protein, or mutation of regulatory sequences which affect gene expression. Deletion mutants may be preferred because of the accompanying low probability of reversion of the auxotrophic phenotype. As an example, mutants of D-alanine which are generated according to the protocols presented herein may be tested for the ability to grow in the absence of D-alanine in a simple laboratory culture assay. Those mutants which are unable to grow in the absence of this compound can be selected.

Examples of endogenous amino acid metabolism genes include a vitamin synthesis gene, a gene encoding pantothenic acid synthase, a D-glutamic acid synthase gene, a D-alanine amino transferase (dat) gene, a D-alanine racemase (dal) gene, dga, a gene involved in the synthesis of diaminopimelic acid (DAP), a gene involved in the synthesis of Cysteine synthase A (cysK), a vitamin-B12 independent methionine synthase, trpA, trpB, trpE, asnB, gltD, gltB, leuA, argG, and thrC. The Listeria strain can be deficient in two or more such genes (e.g., dat and dal). D-glutamic acid synthesis is controlled in part by the dal gene, which is involved in the conversion of D-glu+pyr to alpha-ketoglutarate+D-ala, and the reverse reaction.

As another example, an attenuated Listeria strain can be deficient in an endogenous synthase gene, such as an amino acid synthesis gene. Examples of such genes include folP, a gene encoding a dihydrouridine synthase family protein, ispD, ispF, a gene encoding a phosphoenolpyruvate synthase, hisF, hisH, fliI, a gene encoding a ribosomal large subunit pseudouridine synthase, ispD, a gene encoding a bifunctional GMP synthase/glutamine amidotransferase protein, cobS, cobB, cbiD, a gene encoding a uroporphyrin-III C-methyltransferase/uroporphyrinogen-III synthase, cobQ, uppS, truB, dxs, mvaS, dapA, ispG, folC, a gene encoding a citrate synthase, argJ, a gene encoding a 3-deoxy-7-phosphoheptulonate synthase, a gene encoding an indole-3-glycerol-phosphate synthase, a gene encoding an anthranilate synthase/glutamine amidotransferase component, menB, a gene encoding a menaquinone-specific isochorismate synthase, a gene encoding a phosphoribosylformylglycinamidine synthase I or II, a gene encoding a phosphoribosylaminoimidazole-succinocarboxamide synthase, carB, carA, thyA, mgsA, aroB, hepB, rluB, ilvB, ilvN, alsS, fabF, fabH, a gene encoding a pseudouridine synthase, pyrG, truA, pabB, and an atp synthase gene (e.g., atpC, atpD-2, aptG, atpA-2, and so forth).

Attenuated Listeria strains can be deficient in endogenous phoP, aroA, aroC, aroD, or plcB. As yet another example, an attenuated Listeria strain can be deficient in an endogenous peptide transporter. Examples include genes encoding an ABC transporter/ATP-binding/permease protein, an oligopeptide ABC transporter/oligopeptide-binding protein, an oligopeptide ABC transporter/permease protein, a zinc ABC transporter/zinc-binding protein, a sugar ABC transporter, a phosphate transporter, a ZIP zinc transporter, a drug resistance transporter of the EmrB/QacA family, a sulfate transporter, a proton-dependent oligopeptide transporter, a magnesium transporter, a formate/nitrite transporter, a spermidine/putrescine ABC transporter, a Na/Pi-cotransporter, a sugar phosphate transporter, a glutamine ABC transporter, a major facilitator family transporter, a glycine betaine/L-proline ABC transporter, a molybdenum ABC transporter, a techoic acid ABC transporter, a cobalt ABC transporter, an ammonium transporter, an amino acid ABC transporter, a cell division ABC transporter, a manganese ABC transporter, an iron compound ABC transporter, a maltose/maltodextrin ABC transporter, a drug resistance transporter of the Bcr/CflA family, and a subunit of one of the above proteins.

Other attenuated bacteria and Listeria strains can be deficient in an endogenous metabolic enzyme that metabolizes an amino acid that is used for a bacterial growth process, a replication process, cell wall synthesis, protein synthesis, metabolism of a fatty acid, or for any other growth or replication process. Likewise, an attenuated strain can be deficient in an endogenous metabolic enzyme that can catalyze the formation of an amino acid used in cell wall synthesis, can catalyze the synthesis of an amino acid used in cell wall synthesis, or can be involved in synthesis of an amino acid used in cell wall synthesis. Alternatively, the amino acid can be used in cell wall biogenesis. Alternatively, the metabolic enzyme is a synthetic enzyme for D-glutamic acid, a cell wall component.

Other attenuated Listeria strains can be deficient in metabolic enzymes encoded by a D-glutamic acid synthesis gene, dga, an alr (alanine racemase) gene, or any other enzymes that are involved in alanine synthesis. Yet other examples of metabolic enzymes for which the Listeria strain can be deficient include enzymes encoded by serC (a phosphoserine aminotransferase), asd (aspartate betasemialdehyde dehydrogenase; involved in synthesis of the cell wall constituent diaminopimelic acid), the gene encoding gsaB—glutamate-1-semialdehyde aminotransferase (catalyzes the formation of 5-aminolevulinate from (S)-4-amino-5-oxopentanoate), hemL (catalyzes the formation of 5-aminolevulinate from (S)-4-amino-5-oxopentanoate), aspB (an aspartate aminotransferase that catalyzes the formation of oxaloacetate and L-glutamate from L-aspartate and 2-oxoglutarate), argF-1 (involved in arginine biosynthesis), aroE (involved in amino acid biosynthesis), aroB (involved in 3-dehydroquinate biosynthesis), aroD (involved in amino acid biosynthesis), aroC (involved in amino acid biosynthesis), hisB (involved in histidine biosynthesis), hisD (involved in histidine biosynthesis), hisG (involved in histidine biosynthesis), metX (involved in methionine biosynthesis), proB (involved in proline biosynthesis), argR (involved in arginine biosynthesis), argJ (involved in arginine biosynthesis), thil (involved in thiamine biosynthesis), LMOf2365_1652 (involved in tryptophan biosynthesis), aroA (involved in tryptophan biosynthesis), ilvD (involved in valine and isoleucine biosynthesis), ilvC (involved in valine and isoleucine biosynthesis), leuA (involved in leucine biosynthesis), dapF (involved in lysine biosynthesis), and thrB (involved in threonine biosynthesis) (all GenBank Accession No. NC_002973).

An attenuated Listeria strain can be generated by mutation of other metabolic enzymes, such as a tRNA synthetase. For example, the metabolic enzyme can be encoded by the trpS gene, encoding tryptophanyl tRNA synthetase. For example, the host strain bacteria can be Δ(trpS aroA), and both markers can be contained in an integration vector.

Other examples of metabolic enzymes that can be mutated to generate an attenuated Listeria strain include an enzyme encoded by murE (involved in synthesis of diaminopimelic acid; GenBank Accession No: NC_003485), LMOf2365_2494 (involved in teichoic acid biosynthesis), WecE (Lipopolysaccharide biosynthesis protein rffA; GenBank Accession No: AE014075.1), or amiA (an N-acetylmuramoyl-L-alanine amidase). Yet other examples of metabolic enzymes include aspartate aminotransferase, histidinol-phosphate aminotransferase (GenBank Accession No. NP_466347), or the cell wall teichoic acid glycosylation protein GtcA.

Other examples of metabolic enzymes that can be mutated to generate an attenuated Listeria strain include a synthetic enzyme for a peptidoglycan component or precursor. The component can be, for example, UDP-N-acetylmuramylpentapeptide, UDP-N-acetylglucosamine, MurNAc-(pentapeptide)-pyrophosphoryl-undecaprenol, GlcNAc-p-(1,4)-MurNAc-(pentapeptide)-pyrophosphorylundecaprenol, or any other peptidoglycan component or precursor.

Yet other examples of metabolic enzymes that can be mutated to generate an attenuated Listeria strain include metabolic enzymes encoded by murG, murD, murA-1, or murA-2 (all set forth in GenBank Accession No. NC_002973). Alternatively, the metabolic enzyme can be any other synthetic enzyme for a peptidoglycan component or precursor. The metabolic enzyme can also be a trans-glycosylase, a trans-peptidase, a carboxy-peptidase, any other class of metabolic enzyme, or any other metabolic enzyme. For example, the metabolic enzyme can be any other Listeria metabolic enzyme or any other Listeria monocytogenes metabolic enzyme.

Other bacterial strains can be attenuated as described above for Listeria by mutating the corresponding orthologous genes in the other bacterial strains.

(2) Methods of Complementing Attenuated Bacteria and Listeria Strains

The attenuated bacteria or Listeria strains disclosed herein can further comprise a nucleic acid comprising a complementing gene or encoding a metabolic enzyme that complements an attenuating mutation (e.g., complements the auxotrophy of the auxotrophic Listeria strain). For example, a nucleic acid having a first open reading frame encoding a fusion polypeptide or chimeric polypeptide as disclosed herein can further comprise a second open reading frame comprising the complementing gene or encoding the complementing metabolic enzyme. Alternatively, a first nucleic acid can encode the fusion polypeptide or chimeric polypeptide and a separate second nucleic acid can comprise the complementing gene or encode the complementing metabolic enzyme.

The complementing gene can be extrachromosomal or can be integrated into the bacteria or Listeria genome. For example, the auxotrophic Listeria strain can comprise an episomal plasmid comprising a nucleic acid encoding a metabolic enzyme. Such plasmids will be contained in the Listeria in an episomal or extrachromosomal fashion. Alternatively, the auxotrophic Listeria strain can comprise an integrative plasmid (i.e., integration vector) comprising a nucleic acid encoding a metabolic enzyme. Such integrative plasmids can be used for integration into a Listeria chromosome. Preferably, the episomal plasmid or the integrative plasmid lacks an antibiotic resistance marker.

The metabolic gene can be used for selection instead of or in addition to an antibiotic resistance gene. As an example, in order to select for auxotrophic bacteria comprising a plasmid encoding a metabolic enzyme or a complementing gene provided herein, transformed auxotrophic bacteria can be grown in a medium that will select for expression of the gene encoding the metabolic enzyme (e.g., amino acid metabolism gene) or the complementing gene. For example, a bacteria auxotrophic for D-glutamic acid synthesis can be transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. Similarly, a bacterium auxotrophic for D-alanine synthesis will grow in the absence of D-alanine when transformed and expressing a plasmid comprising a nucleic acid encoding an amino acid metabolism enzyme for D-alanine synthesis. Such methods for making appropriate media comprising or lacking necessary growth factors, supplements, amino acids, vitamins, antibiotics, and the like are well-known and are available commercially.

Once the auxotrophic bacteria comprising the plasmid encoding a metabolic enzyme or a complementing gene provided herein have been selected in appropriate medium, the bacteria can be propagated in the presence of a selective pressure. Such propagation can comprise growing the bacteria in media without the auxotrophic factor. The presence of the plasmid expressing the metabolic enzyme or the complementing gene in the auxotrophic bacteria ensures that the plasmid will replicate along with the bacteria, thus continually selecting for bacteria harboring the plasmid. Production of the bacteria or Listeria strain can be readily scaled up by adjusting the volume of the medium in which the auxotrophic bacteria comprising the plasmid are growing.

In one specific example, the attenuated strain is a strain having a deletion of or an inactivating mutation in dal and dat (e.g., Listeria monocytogenes (Lm) dal(−)dat(−) (Lmdd) or Lm dal(−)dat(−)ΔactA (LmddA)), and the complementing gene encodes an alanine racemase enzyme (e.g., encoded by dal gene) or a D-amino acid aminotransferase enzyme (e.g., encoded by dat gene). An exemplary alanine racemase protein can have the sequence set forth in SEQ ID NO: 76 (encoded by SEQ ID NO: 78; GenBank Accession No: AF038438) or can be a homologue, variant, isoform, analog, fragment, fragment of a homologue, fragment of a variant, fragment of an analog, or fragment of an isoform of SEQ ID NO: 76. The alanine racemase protein can also be any other Listeria alanine racemase protein. Alternatively, the alanine racemase protein can be any other gram-positive alanine racemase protein or any other alanine racemase protein. An exemplary D-amino acid aminotransferase protein can have the sequence set forth in SEQ ID NO: 77 (encoded by SEQ ID NO: 79; GenBank Accession No: AF038439) or can be a homologue, variant, isoform, analog, fragment, fragment of a homologue, fragment of a variant, fragment of an analog, or fragment of an isoform of SEQ ID NO: 77. The D-amino acid aminotransferase protein can also be any other Listeria D-amino acid aminotransferase protein. Alternatively, the D-amino acid aminotransferase protein can be any other gram-positive D-amino acid aminotransferase protein or any other D-amino acid aminotransferase protein.

In another specific example, the attenuated strain is a strain having a deletion of or an inactivating mutation in prfA (e.g., Lm prfA(−)), and the complementing gene encodes a PrfA protein. For example, the complementing gene can encode a mutant PrfA (D133V) protein that restores partial PrfA function. An example of a wild type PrfA protein is set forth in SEQ ID NO: 80 (encoded by nucleic acid set forth in SEQ ID NO: 81), and an example of a D133V mutant PrfA protein is set forth in SEQ ID NO: 82 (encoded by nucleic acid set forth in SEQ ID NO: 83). The complementing PrfA protein can be a homologue, variant, isoform, analog, fragment, fragment of a homologue, fragment of a variant, fragment of an analog, or fragment of an isoform of SEQ ID NO: 80 or 82. The PrfA protein can also be any other Listeria PrfA protein. Alternatively, the PrfA protein can be any other gram-positive PrfA protein or any other PrfA protein.

In another example, the bacteria strain or Listeria strain can comprise a deletion of or an inactivating mutation in an actA gene, and the complementing gene can comprise an actA gene to complement the mutation and restore function to the Listeria strain.

Other auxotroph strains and complementation systems can also be adopted for the use with the methods and compositions provided herein.

C. Preparation and Storage of Bacteria or Listeria Strains

The recombinant bacteria strain (e.g., Listeria strain) optionally has been passaged through an animal host. Such passaging can maximize efficacy of the Listeria strain as a vaccine vector, can stabilize the immunogenicity of the Listeria strain, can stabilize the virulence of the Listeria strain, can increase the immunogenicity of the Listeria strain, can increase the virulence of the Listeria strain, can remove unstable sub-strains of the Listeria strain, or can reduce the prevalence of unstable sub-strains of the Listeria strain. Methods for passaging a recombinant Listeria strain through an animal host are well known in the art and are described, for example, in US 2006/0233835, herein incorporated by reference in its entirety for all purposes.

The recombinant bacteria strain (e.g., Listeria strain) can be stored in a frozen cell bank or stored in a lyophilized cell bank. Such a cell bank can be, for example, a master cell bank, a working cell bank, or a Good Manufacturing Practice (GMP) cell bank. Examples of “Good Manufacturing Practices” include those defined by 21 CFR 210-211 of the United States Code of Federal Regulations. However, “Good Manufacturing Practices” can also be defined by other standards for production of clinical-grade material or for human consumption, such as standards of a country other than the United States. Such cell banks can be intended for production of clinical-grade material or can conform to regulatory practices for human use.

Recombinant bacteria strains (e.g., Listeria strains) can also be from a batch of vaccine doses, from a frozen stock, or from a lyophilized stock.

Such cell banks, frozen stocks, or batches of vaccine doses can, for example, exhibit viability upon thawing of greater than 90%. The thawing, for example, can follow storage for cryopreservation or frozen storage for 24 hours. Alternatively, the storage can last, for example, for 2 days, 3 days, 4 days, 1 week, 2 weeks, 3 weeks, 1 month, 2 months, 3 months, 5 months, 6 months, 9 months, or 1 year.

The cell bank, frozen stock, or batch of vaccine doses can be cryopreserved, for example, by a method that comprises growing a culture of the bacteria strain (e.g., Listeria strain) in a nutrient media, freezing the culture in a solution comprising glycerol, and storing the Listeria strain at below −20° C. The temperature can be, for example, about −70° C. or between about −70 to about −80° C. Alternatively, the cell bank, frozen stock, or batch of vaccine doses can be cryopreserved by a method that comprises growing a culture of the Listeria strain in a defined medium, freezing the culture in a solution comprising glycerol, and storing the Listeria strain at below −20° C. The temperature can be, for example, about −70° C. or between about −70 to about −80° C. Any defined microbiological medium may be used in this method.

The culture (e.g., the culture of a Listeria vaccine strain that is used to produce a batch of Listeria vaccine doses) can be inoculated, for example, from a cell bank, from a frozen stock, from a starter culture, or from a colony. The culture can be inoculated, for example, at mid-log growth phase, at approximately mid-log growth phase, or at another growth phase.

The solution used for freezing optionally contain another colligative additive or additive with anti-freeze properties in place of glycerol or in addition to glycerol. Examples of such additives include, for example, mannitol, DMSO, sucrose, or any other colligative additive or additive with anti-freeze properties.

The nutrient medium utilized for growing a culture of a bacteria strain (e.g., a Listeria strain) can be any suitable nutrient medium. Examples of suitable media include, for example, LB; TB; a modified, animal-product-free Terrific Broth; or a defined medium.

The step of growing can be performed by any known means of growing bacteria. For example, the step of growing can be performed with a shake flask (such as a baffled shake flask), a batch fermenter, a stirred tank or flask, an airlift fermenter, a fed batch, a continuous cell reactor, an immobilized cell reactor, or any other means of growing bacteria.

Optionally, a constant pH is maintained during growth of the culture (e.g. in a batch fermenter). For example, the pH can be maintained at about 6.0, at about 6.5, at about 7.0, at about 7.5, or about 8.0. Likewise, the pH can be, for example, from about 6.5 to about 7.5, from about 6.0 to about 8.0, from about 6.0 to about 7.0, from about 6.0 to about 7.0, or from about 6.5 to about 7.5.

Optionally, a constant temperature can be maintained during growth of the culture. For example, the temperature can be maintained at about 37° C. or at 37° C. Alternatively, the temperature can be maintained at 25° C., 27° C., 28° C., 30° C., 32° C., 34° C., 35° C., 36° C., 38° C., or 39° C.

Optionally, a constant dissolved oxygen concentration can be maintained during growth of the culture. For example, the dissolved oxygen concentration can be maintained at 20% of saturation, 15% of saturation, 16% of saturation, 18% of saturation, 22% of saturation, 25% of saturation, 30% of saturation, 35% of saturation, 40% of saturation, 45% of saturation, 50% of saturation, 55% of saturation, 60% of saturation, 65% of saturation, 70% of saturation, 75% of saturation, 80% of saturation, 85% of saturation, 90% of saturation, 95% of saturation, 100% of saturation, or near 100% of saturation.

Methods for lyophilization and cryopreservation of recombinant bacteria strains (e.g., Listeria strains are known. For example, a Listeria culture can be flash-frozen in liquid nitrogen, followed by storage at the final freezing temperature. Alternatively, the culture can be frozen in a more gradual manner (e.g., by placing in a vial of the culture in the final storage temperature). The culture can also be frozen by any other known method for freezing a bacterial culture.

The storage temperature of the culture can be, for example, between −20 and −80° C. For example, the temperature can be significantly below −20° C. or not warmer than −70° C. Alternatively, the temperature can be about −70° C., −20° C., −30° C., −40° C., −50° C., −60° C., −80° C., −30 to −70° C., −40 to −70° C., −50 to −70° C., −60 to −70° C., −30 to −80° C., −40 to −80° C., −50 to −80° C., −60 to −80° C., or −70 to −80° C. Alternatively, the temperature can be colder than 70° C. or colder than −80° C.

### V. Immunogenic Compositions, Pharmaceutical Compositions, and Vaccines

Also provided are immunogenic compositions, pharmaceutical compositions, or vaccines comprising a recombinant fusion polypeptide or chimeric polypeptide as disclosed herein, a nucleic acid encoding a recombinant fusion polypeptide or chimeric polypeptide (e.g., minigene construct) as disclosed herein, or a recombinant bacteria or Listeria strain as disclosed herein. An immunogenic composition comprising a Listeria strain can be inherently immunogenic by virtue of its comprising a Listeria strain and/or the composition can also further comprise an adjuvant. Other immunogenic compositions comprise DNA immunotherapy or peptide immunotherapy compositions.

The term “immunogenic composition” refers to any composition containing an antigen that elicits an immune response against the antigen in a subject upon exposure to the composition. The immune response elicited by an immunogenic composition can be to a particular antigen or to a particular epitope on the antigen.

An immunogenic composition can comprise a single recombinant fusion polypeptide or chimeric polypeptide as disclosed herein, nucleic acid encoding a recombinant fusion polypeptide or chimeric polypeptide (e.g., minigene construct) as disclosed herein, or recombinant bacteria or Listeria strain as disclosed herein, or it can comprise multiple different recombinant fusion polypeptides or chimeric polypeptides as disclosed herein, nucleic acids encoding recombinant fusion polypeptides or chimeric polypeptides (e.g., minigene constructs) as disclosed herein, or recombinant bacteria or Listeria strains as disclosed herein. A first recombinant fusion polypeptide or chimeric polypeptide is different from a second recombinant fusion polypeptide or chimeric polypeptide, for example, if it includes one antigenic peptide that the second recombinant fusion polypeptide or chimeric polypeptide does not. The two recombinant fusion polypeptides or chimeric polypeptides can include some of the same antigenic peptides and still be considered different. Such different recombinant fusion polypeptides or chimeric polypeptides, nucleic acids encoding recombinant fusion polypeptides or chimeric polypeptides, or recombinant bacteria or Listeria strains can be administered concomitantly to a subject or sequentially to a subject. Sequential administration can be particularly useful when a drug substance comprising a recombinant Listeria strain (or recombinant fusion polypeptide or chimeric polypeptide or nucleic acid) disclosed herein is in different dosage forms (e.g., one agent is a tablet or capsule and another agent is a sterile liquid) and/or is administered on different dosing schedules (e.g., one composition from the mixture is administered at least daily and another is administered less frequently, such as once weekly, once every two weeks, or once every three weeks). The multiple recombinant fusion polypeptides or chimeric polypeptides, nucleic acids encoding recombinant fusion polypeptides or chimeric polypeptides, or recombinant bacteria or Listeria strains can each comprise a different set of antigenic peptides. Alternatively, two or more of the recombinant fusion polypeptides or chimeric polypeptides, nucleic acids encoding recombinant fusion polypeptides or chimeric polypeptides, or recombinant bacteria or Listeria strains can comprise the same set of antigenic peptides (e.g., the same set of antigenic peptides in a different order).

An immunogenic composition can additionally comprise an adjuvant (e.g., two or more adjuvants), a cytokine, a chemokine, or combination thereof. Optionally, an immunogenic composition can additionally comprises antigen presenting cells (APCs), which can be autologous or can be allogeneic to the subject.

The term adjuvant includes compounds or mixtures that enhance the immune response to an antigen. For example, an adjuvant can be a non-specific stimulator of an immune response or substances that allow generation of a depot in a subject which when combined with an immunogenic composition disclosed herein provides for an even more enhanced and/or prolonged immune response. An adjuvant can favor, for example, a predominantly Th1-mediated immune response, a Th1-type immune response, or a Th1-mediated immune response. Likewise, an adjuvant can favor a cell-mediated immune response over an antibody-mediated response. Alternatively, an adjuvant can favor an antibody-mediated response. Some adjuvants can enhance the immune response by slowly releasing the antigen, while other adjuvants can mediate their effects by any of the following mechanisms: increasing cellular infiltration, inflammation, and trafficking to the injection site, particularly for antigen-presenting cells (APC); promoting the activation state of APCs by upregulating costimulatory signals or major histocompatibility complex (MHC) expression; enhancing antigen presentation; or inducing cytokine release for indirect effect.

Examples of adjuvants include saponin QS21, CpG oligonucleotides, unmethylated CpG-containing oligonucleotides, MPL, TLR agonists, TLR4 agonists, TLR9 agonists, Resiquimod®, imiquimod, cytokines or nucleic acids encoding the same, chemokines or nucleic acids encoding same, IL-12 or a nucleic acid encoding the same, IL-6 or a nucleic acid encoding the same, and lipopolysaccharides. Another example of a suitable adjuvant is Montanide ISA 51. Montanide ISA 51 contains a natural metabolizable oil and a refined emulsifier. Other examples of a suitable adjuvant include granulocyte/macrophage colony-stimulating factor (GM-CSF) or a nucleic acid encoding the same and keyhole limpet hemocyanin (KLH) proteins or nucleic acids encoding the same. The GM-CSF can be, for example, a human protein grown in a yeast (S. cerevisiae) vector. GM-CSF promotes clonal expansion and differentiation of hematopoietic progenitor cells, antigen presenting cells (APCs), dendritic cells, and T cells. Yet another example of a suitable adjuvant is detoxified listeriolysin O (dtLLO) protein. One example of a dtLLO suitable for use as an adjuvant is encoded by SEQ ID NO: 149. A dtLLO encoded by a sequence at least 90%, 95%, 96%, 97%, 98%, or 99% identical to SEQ ID NO: 149 is also suitable for use as an adjuvant. Yet other examples of adjuvants include growth factors or nucleic acids encoding the same, cell populations, Freund's incomplete adjuvant, aluminum phosphate, aluminum hydroxide, BCG (bacille Calmette-Guerin), alum, interleukins or nucleic acids encoding the same, quill glycosides, monophosphoryl lipid A, liposomes, bacterial mitogens, bacterial toxins, or any other type of known adjuvant (see, e.g., Fundamental Immunology, 5th ed. (August 2003): William E. Paul (Editor); Lippincott Williams & Wilkins Publishers; Chapter 43: Vaccines, GJV Nossal, which is herein incorporated by reference in its entirety for all purposes).

An immunogenic composition can further comprise one or more immunomodulatory molecules. Examples include interferon gamma, a cytokine, a chemokine, and a T cell stimulant.

An immunogenic composition can be in the form of a vaccine or pharmaceutical composition. The terms “vaccine” and “pharmaceutical composition” are interchangeable and refer to an immunogenic composition in a pharmaceutically acceptable carrier for in vivo administration to a subject. A vaccine may be, for example, a peptide vaccine (e.g., comprising a recombinant fusion polypeptide or chimeric polypeptide as disclosed herein), a DNA vaccine (e.g., comprising a nucleic acid encoding a recombinant fusion polypeptide or chimeric polypeptide as disclosed herein), or a vaccine contained within and delivered by a cell (e.g., a recombinant Listeria as disclosed herein). A vaccine may prevent a subject from contracting or developing a disease or condition and/or a vaccine may be therapeutic to a subject having a disease or condition. Methods for preparing peptide vaccines are well known and are described, for example, in EP 1408048, US 2007/0154953, and Ogasawara et al. (1992) Proc. Natl Acad Sci USA 89:8995-8999, each of which is herein incorporated by reference in its entirety for all purposes. Optionally, peptide evolution techniques can be used to create an antigen with higher immunogenicity. Techniques for peptide evolution are well known and are described, for example, in U.S. Pat. No. 6,773,900, herein incorporated by reference in its entirety for all purposes.

A “pharmaceutically acceptable carrier” refers to a vehicle for containing an immunogenic composition that can be introduced into a subject without significant adverse effects and without having deleterious effects on the immunogenic composition. That is, “pharmaceutically acceptable” refers to any formulation which is safe, and provides the appropriate delivery for the desired route of administration of an effective amount of at least one immunogenic composition for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the immunogenic compositions and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with the immunogenic composition.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

An immunogenic composition, pharmaceutical composition, or vaccine disclosed herein may also comprise one or more additional compounds effective in preventing or treating cancer. For example, the additional compound may comprise a compound useful in chemotherapy, such as amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil (5-FU), gemcitabine, gliadelimplants, hydroxycarbamide, idarubicin, ifosfamide, irinotecan, leucovorin, liposomaldoxorubicin, liposomaldaunorubicin, lomustine, melphalan, mercaptopurine, mesna, methotrexate, mitomycin, mitoxantrone, oxaliplatin, paclitaxel (Taxol), pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. The additional compound can also comprise other biologics, including Herceptin® (trastuzumab) against the HER2 antigen, Avastin® (bevacizumab) against VEGF, or antibodies to the EGF receptor, such as Erbitux® (cetuximab), and Vectibix® (panitumumab). The additional compound can also comprise, for example, an additional immunotherapy.

An additional compound can also comprise an immune checkpoint inhibitor antagonist, such as a PD-1 signaling pathway inhibitor, a CD-80/86 and CTLA-4 signaling pathway inhibitor, a T cell membrane protein 3 (TIM3) signaling pathway inhibitor, an adenosine A2a receptor (A2aR) signaling pathway inhibitor, a lymphocyte activation gene 3 (LAG3) signaling pathway inhibitor, a killer immunoglobulin receptor (KIR) signaling pathway inhibitor, a CD40 signaling pathway inhibitor, or any other antigen-presenting cell/T cell signaling pathway inhibitor. Examples of immune checkpoint inhibitor antagonists include an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof. An additional compound can also comprise a T cell stimulator, such as an antibody or functional fragment thereof binding to a T-cell receptor co-stimulatory molecule, an antigen presenting cell receptor binding co-stimulatory molecule, or a member of the TNF receptor superfamily. The T-cell receptor co-stimulatory molecule can comprise, for example, CD28 or ICOS. The antigen presenting cell receptor binding co-stimulatory molecule can comprise, for example, a CD80 receptor, a CD86 receptor, or a CD46 receptor. The TNF receptor superfamily member can comprise, for example, glucocorticoid-induced TNF receptor (GITR), OX40 (CD134 receptor), 4-1BB (CD137 receptor), or TNFR25. See, e.g., WO2016100929, WO2016011362, and WO2016011357, each of which is incorporated by reference in its entirety for all purposes.

### VI. Therapeutic Methods

The recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, and vaccines disclosed herein can be used in various methods. For example, they can be used in methods of inducing or enhancing an anti-WT1 immune response in a subject, in methods of inducing or enhancing an anti-WT1-expressing-tumor or anti-WT1-expressing-cancer immune response in a subject, in methods of treating a tumor or cancer in a subject, in methods of preventing a tumor or cancer in a subject, or in methods of protecting a subject against a tumor or cancer. They can also be used in methods of increasing the ratio of T effector cells to regulatory T cells (Tregs) in the spleen and tumor of a subject, wherein the T effector cells are targeted to a WT1 antigen. They can also be used in methods for increasing WT1 T cells in a subject, increasing survival time of a subject having a tumor or cancer, delaying the onset of cancer in a subject, or reducing tumor or metastasis size in a subject.

A method of inducing an anti-Wilms-tumor-protein immune response in a subject can comprise, for example, administering to the subject a recombinant fusion polypeptide or chimeric polypeptide, a nucleic acid encoding a recombinant fusion polypeptide or chimeric polypeptide (e.g., a minigene construct), a recombinant bacteria or Listeria strain, an immunogenic composition, a pharmaceutical composition, or a vaccine disclosed herein (e.g., that comprises a recombinant fusion polypeptide or chimeric polypeptide disclosed elsewhere herein or a nucleic acid encoding the recombinant fusion polypeptide or chimeric polypeptide). An anti-Wilms tumor protein immune response can thereby be induced in the subject. For example, in the case of a recombinant Listeria strain, the Listeria strain can express the fusion polypeptide or chimeric polypeptide, thereby eliciting an immune response in the subject. The immune response can comprise, for example, a T-cell response, such as a CD4+FoxP3− T cell response, a CD8+ T cell response, or a CD4+FoxP3− and CD8+ T cell response. Such methods can also increase the ratio of T effector cells to regulatory T cells (Tregs) in the spleen and tumor microenvironments of the subject, allowing for a more profound anti-tumor response in the subject.

A method of inducing an anti-WT1-expressing-tumor or anti-WT1-expressing-cancer immune response in a subject can comprise, for example, administering to the subject a recombinant fusion polypeptide or chimeric polypeptide, a nucleic acid encoding a recombinant fusion polypeptide or chimeric polypeptide (e.g., a minigene construct), a recombinant bacteria or Listeria strain, an immunogenic composition, a pharmaceutical composition, or a vaccine disclosed herein. An anti-tumor or anti-cancer immune response can thereby be induced in the subject. For example, in the case of a recombinant Listeria strain, the Listeria strain can express the fusion polypeptide or chimeric polypeptide, thereby eliciting an anti-tumor or anti-cancer response in the subject.

A method of treating a tumor or cancer in a subject (e.g., wherein the tumor or cancer expresses a Wilms tumor protein), can comprise, for example, administering to the subject a recombinant fusion polypeptide or chimeric polypeptide, a nucleic acid encoding a recombinant fusion polypeptide or chimeric polypeptide (e.g., a minigene construct), a recombinant bacteria or Listeria strain, an immunogenic composition, a pharmaceutical composition, or a vaccine disclosed herein. The subject can then mount an immune response against the tumor or cancer expressing the Wilms tumor protein, thereby treating the tumor or cancer in the subject.

A method of preventing a tumor or cancer in a subject or protecting a subject against developing a tumor or cancer (e.g., wherein the tumor or cancer is associated with expression of Wilms tumor protein), can comprise, for example, administering to the subject a recombinant fusion polypeptide or chimeric polypeptide, a nucleic acid encoding a recombinant fusion polypeptide or chimeric polypeptide (e.g., a minigene construct), a recombinant bacteria or Listeria strain, an immunogenic composition, a pharmaceutical composition, or a vaccine disclosed herein. The subject can then mount an immune response against the Wilms tumor protein, thereby preventing a tumor or cancer or protecting the subject against developing a tumor or cancer.

In some of the above methods, two or more recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines are administered. The multiple recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines can be administered sequentially in any order or combination, or can be administered simultaneously in any combination. As an example, if four different Listeria strains are being administered, they can be administered sequentially, they can be administered simultaneously, or they can be administered in any combination (e.g., administering the first and second strains simultaneously and subsequently administering the third and fourth strains simultaneously). Optionally, in the case of sequential administration, the compositions can be administered during the same immune response, preferably within 0-10 or 3-7 days of each other. The multiple recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines can each comprise a different set of antigenic peptides. Alternatively, two or more can comprise the same set of antigenic peptides (e.g., the same set of antigenic peptides in a different order).

Cancer is a physiological condition in mammals that is typically characterized by unregulated cell growth and proliferation. Any cancer or pre-malignant condition expressing or associated with WT1 expression can be the subject of the therapeutic or prophylactic methods disclosed herein (i.e., any hematologic or solid tumor malignancy as well as any pre-malignant dysplastic tissue or conditions). Cancers can be hematopoietic malignancies or solid tumors (i.e., masses of cells that result from excessive cell growth or proliferation, including pre-cancerous legions). Metastatic cancer refers to a cancer that has spread from the place where it first started to another place in the body. Tumors formed by metastatic cancer cells are called a metastatic tumor or a metastasis, which is a term also used to refer to the process by which cancer cells spread to other parts of the body. In general, metastatic cancer has the same name and same type of cancer cells as the original, or primary, cancer. Examples of solid tumors include melanoma, carcinoma, blastoma, and sarcoma. Hematologic malignancies include, for example, leukemia or lymphoid malignancies, such as lymphoma. Exemplary categories of cancers include brain, breast, gastrointestinal, genitourinary, gynecologic, head and neck, heme, skin and thoracic. Brain malignancies include, for example, glioblastoma, high-grade pontine glioma, low-grade glioma, medulloblastoma, neuroblastoma, and pilocytic astrocytoma. Gastrointestinal cancers include, for example, colorectal, gallbladder, hepatocellular, pancreas, PNET, gastric, and esophageal. Genitourinary cancers include, for example, adrenocortical, bladder, kidney chromophobe, renal (clear cell), renal (papillary), rhabdoid cancers, and prostate. Gynecologic cancers include, for example, uterine carcinosarcoma, uterine endometrial, serous ovarian, and cervical. Head and neck cancers include, for example, thyroid, nasopharyngeal, head and neck, and adenoid cystic. Heme cancers include, for example, multiple myeloma, myelodysplasia, mantle-cell lymphoma, acute lymphoblastic leukemia (ALL), non-lymphoma, chronic lymphocytic leukemia (CLL), and acute myeloid leukemia (AML). Skin cancers includes, for example, cutaneous melanoma and squamous cell carcinoma. Thoracic cancers include, for example, squamous lung, small-cell lung, and lung adenocarcinoma.

More particular examples of such cancers include squamous cell cancer or carcinoma (e.g., oral squamous cell carcinoma), myeloma, oral cancer, juvenile nasopharyngeal angiofibroma, neuroendocrine tumors, lung cancer, cancer of the peritoneum, hepatocellular cancer, gastric or stomach cancer including gastrointestinal cancer, pancreatic cancer, glioma, glioblastoma, glial tumors, cervical cancer, ovarian cancer, liver cancer, bladder cancer, hepatoma, hepatocellular carcinoma, breast cancer, triple-negative breast cancer, colon cancer, rectal cancer, colorectal cancer, endometrial cancer or uterine cancer or carcinoma, salivary gland carcinoma, kidney or renal cancer (e.g., renal cell carcinoma), prostate cancer, vulval cancer, thyroid cancer, hepatic carcinoma, anal carcinoma, penile carcinoma, fibrosarcoma, gallbladder cancer, osteosarcoma, mesothelioma, as well as head and neck cancer. A cancer can also be a brain cancer or another type of CNS or intracranial tumor. For example, a subject can have an astrocytic tumor (e.g., astrocytoma, anaplastic astrocytoma, glioblastoma, pilocytic astrocytoma, subependymal giant cell astrocytoma, pleomorphic xanthoastrocytoma), oligodendroglial tumor (e.g., oligodendroglioma, anaplastic oligodendroglioma), ependymal cell tumor (e.g., ependymoma, anaplastic ependymoma, myxopapillary ependymoma, subependymoma), mixed glioma (e.g., mixed oligoastrocytoma, anaplastic oligoastrocytoma), neuroepithelial tumor of uncertain origin (e.g., polar spongioblastoma, astroblastoma, gliomatosis cerebri), tumor of the choroid plexus (e.g., choroid plexus papilloma, choroid plexus carcinoma), neuronal or mixed neuronal-glial tumor (e.g., gangliocytoma, dyplastic gangliocytoma of cerebellum, ganglioglioma, anaplastic ganglioglioma, desmoplastic infantile ganglioma, central neurocytoma, dysembryoplastic neuroepthelial tumor, olfactory neuroblastoma), pineal parenchyma tumor (e.g., pineocytoma, pineoblastoma, mixed pineocytoma/pineoblastoma), or tumor with mixed neuroblastic or glioblastic elements (e.g., medulloepithelioma, medulloblastoma, neuroblastoma, retinoblastoma, ependymoblastoma).

Some specific examples of types of cancer known to be associated with expression of Wilms tumor protein include Wilms tumor (a pediatric nephroblastoma), breast cancers such as triple negative breast cancer (TNBC) and gastrointestinal cancers such as esophageal cancer, stomach cancer, gastric cancer, pancreatic cancer, liver cancer, gall bladder cancer, colorectal cancer, anal cancer, and gastrointestinal carcinoid tumors. Triple negative breast cancer (TNBC) refers to any breast cancer that does not express the genes for estrogen receptor (ER), progesterone receptor (PR), or Her2/neu. Other examples of cancers that could express WT1 include acute myelogenous leukemia (AML), multiple myeloma, myelodysplastic syndrome (MDS), a cancer associated with MDS, non-small cell lung cancer (NSCLC), a leukemia, a hematological cancer, a lymphoma, a desmoplastic small round cell tumor, a mesothelioma, a malignant mesothelioma, a gastric cancer, a colon cancer, a lung cancer, a breast cancer, a germ cell tumor, an ovarian cancer, a uterine cancer, a thyroid cancer, a hepatocellular carcinoma, a liver cancer, a renal cancer, a Kaposi's sarcoma, or any other carcinoma or sarcoma. A cancer expressing WT1 can be a solid tumor, such as a solid tumor associated with MDS, non-small cell lung cancer (NSCLC), lung cancer, breast cancer, colorectal cancer, prostate cancer, ovarian cancer, renal cancer, pancreatic cancer, brain cancer, gastrointestinal cancer, skin cancer, or melanoma.

The term “treat” or “treating” refers to both therapeutic treatment and prophylactic or preventative measures, wherein the object is to prevent or lessen the targeted tumor or cancer. Treating may include one or more of directly affecting or curing, suppressing, inhibiting, preventing, reducing the severity of, delaying the onset of, slowing the progression of, stabilizing the progression of, inducing remission of, preventing or delaying the metastasis of, reducing/ameliorating symptoms associated with the tumor or cancer, or a combination thereof. For example, treating may include increasing expected survival time or decreasing tumor or metastasis size. The effect (e.g., suppressing, inhibiting, preventing, reducing the severity of, delaying the onset of, slowing the progression of, stabilizing the progression of, inducing remission of, preventing or delaying the metastasis of, reducing/ameliorating symptoms of, and so forth, can be relative to a control subject not receiving a treatment or receiving a placebo treatment. The term “treat” or “treating” can also refer to increasing percent chance of survival or increasing expected time of survival for a subject with the tumor or cancer (e.g., relative to a control subject not receiving a treatment or receiving a placebo treatment). In one example, “treating” refers to delaying progression, expediting remission, inducing remission, augmenting remission, speeding recovery, increasing efficacy of alternative therapeutics, decreasing resistance to alternative therapeutics, or a combination thereof (e.g., relative to a control subject not receiving a treatment or receiving a placebo treatment). The terms “preventing” or “impeding” can refer, for example to delaying the onset of symptoms, preventing relapse of a tumor or cancer, decreasing the number or frequency of relapse episodes, increasing latency between symptomatic episodes, preventing metastasis of a tumor or cancer, or a combination thereof. The terms “suppressing” or “inhibiting” can refer, for example, to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The term “subject” refers to a mammal (e.g., a human) in need of therapy for, or susceptible to developing, a tumor or a cancer. The term subject also refers to a mammal (e.g., a human) that receives either prophylactic or therapeutic treatment. The subject may include dogs, cats, pigs, cows, sheep, goats, horses, rats, mice, non-human mammals, and humans. The term “subject” does not necessarily exclude an individual that is healthy in all respects and does not have or show signs of cancer or a tumor.

An individual is at increased risk of developing a tumor or a cancer if the subject has at least one known risk-factor (e.g., genetic, biochemical, family history, and situational exposure) placing individuals with that risk factor at a statistically significant greater risk of developing the tumor or cancer than individuals without the risk factor.

A “symptom” or “sign” refers to objective evidence of a disease as observed by a physician or subjective evidence of a disease, such as altered gait, as perceived by the subject. A symptom or sign may be any manifestation of a disease. Symptoms can be primary or secondary. The term “primary” refers to a symptom that is a direct result of a particular disease or disorder (e.g., a tumor or cancer), while the term “secondary” refers to a symptom that is derived from or consequent to a primary cause. The recombinant fusion polypeptides or chimeric polypeptides, nucleic acids encoding the recombinant fusion polypeptides or chimeric polypeptides (e.g., minigene constructs), the immunogenic compositions, the pharmaceutical compositions, and the vaccines disclosed herein can treat primary or secondary symptoms or secondary complications.

The recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines are administered in an effective regime, meaning a dosage, route of administration, and frequency of administration that delays the onset, reduces the severity, inhibits further deterioration, and/or ameliorates at least one sign or symptom of the tumor or cancer. Alternatively, the recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines are administered in an effective regime, meaning a dosage, route of administration, and frequency of administration that induces an immune response to a heterologous antigen in the recombinant fusion polypeptide or chimeric polypeptide (or encoded by the nucleic acid), the recombinant bacteria or Listeria strain, the immunogenic composition, the pharmaceutical composition, or the vaccine, or in the case of recombinant bacteria or Listeria strains, that induces an immune response to the bacteria or Listeria strain itself. If a subject is already suffering from the tumor or cancer, the regime can be referred to as a therapeutically effective regime. If the subject is at elevated risk of developing the tumor or cancer relative to the general population but is not yet experiencing symptoms, the regime can be referred to as a prophylactically effective regime. In some instances, therapeutic or prophylactic efficacy can be observed in an individual patient relative to historical controls or past experience in the same patient. In other instances, therapeutic or prophylactic efficacy can be demonstrated in a preclinical or clinical trial in a population of treated patients relative to a control population of untreated patients. For example, a regime can be considered therapeutically or prophylactically effective if an individual treated patient achieves an outcome more favorable than the mean outcome in a control population of comparable patients not treated by methods described herein, or if a more favorable outcome is demonstrated in treated patients versus control patients in a controlled clinical trial (e.g., a phase II, phase II/III or phase III trial) at the p<0.05 or 0.01 or even 0.001 level.

Exemplary dosages for a recombinant Listeria strain are, for example, 1×10⁶-1×10⁷ CFU, 1×10⁷-1×10⁸ CFU, 1×10⁸-3.31×10¹⁰ CFU, 1×10⁹-3.31×10¹⁰ CFU, 5-500×10⁸ CFU, 7-500×10⁸ CFU, 10-500×10⁸ CFU, 20-500×10⁸ CFU, 30-500×10⁸ CFU, 50-500×10⁸ CFU, 70-500×10⁸ CFU, 100-500×10⁸ CFU, 150-500×10⁸ CFU, 5-300×10⁸ CFU, 5-200×10⁸ CFU, 5-15×10⁸ CFU, 5-100×10⁸ CFU, 5-70×10⁸ CFU, 5-50×10⁸ CFU, 5-30×10⁸ CFU, 5-20×10⁸ CFU, 1-30×10⁹ CFU, 1-20×10⁹ CFU, 2-30×10⁹ CFU, 1-10×10⁹ CFU, 2-10×10⁹ CFU, 3-10×10⁹ CFU, 2-7×10⁹ CFU, 2-5×10⁹ CFU, and 3-5×10⁹ CFU. Other exemplary dosages for a recombinant Listeria strain are, for example, 1×10⁷ organisms, 1.5×10⁷ organisms, 2×10⁸ organisms, 3×10⁷ organisms, 4×10⁷ organisms, 5×10⁷ organisms, 6×10⁷ organisms, 7×10⁷ organisms, 8×10⁷ organisms, 10×10⁷ organisms, 1.5×10⁸ organisms, 2×10⁸ organisms, 2.5×10⁸ organisms, 3×10⁸ organisms, 3.3×10⁸ organisms, 4×10⁸ organisms, 5×10⁸ organisms, 1×10⁹ organisms, 1.5×10⁹ organisms, 2×10⁹ organisms, 3×10⁹ organisms, 4×10⁹ organisms, 5×10⁹ organisms, 6×10⁹ organisms, 7×10⁹ organisms, 8×10⁹ organisms, 10×10⁹ organisms, 1.5×10¹⁰ organisms, 2×10¹⁰ organisms, 2.5×10¹⁰ organisms, 3×10¹⁰ organisms, 3.3×10¹⁰ organisms, 4×10¹⁰ organisms, and 5×10¹⁰ organisms. The dosage can depend on the condition of the patient and response to prior treatment, if any, whether the treatment is prophylactic or therapeutic, and other factors.

Administration can be by any suitable means. For example, administration can be parenteral, intravenous, oral, subcutaneous, intra-arterial, intracranial, intrathecal, intracerebroventricular, intraperitoneal, topical, intranasal, intramuscular, intra-ocular, intrarectal, conjunctival, transdermal, intradermal, vaginal, rectal, intratumoral, parcanceral, transmucosal, intravascular, intraventricular, inhalation (aerosol), nasal aspiration (spray), sublingual, aerosol, suppository, or a combination thereof. For intranasal administration or application by inhalation, solutions or suspensions of the recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines mixed and aerosolized or nebulized in the presence of the appropriate carrier are suitable. Such an aerosol may comprise any recombinant fusion polypeptide or chimeric polypeptide, nucleic acids encoding a recombinant fusion polypeptide or chimeric polypeptide (e.g., minigene construct), recombinant bacteria or Listeria strain, immunogenic composition, pharmaceutical composition, or vaccine described herein. Administration may also be in the form of a suppository (e.g., rectal suppository or urethral suppository), in the form of a pellet for subcutaneous implantation (e.g., providing for controlled release over a period of time), or in the form of a capsule. Administration may also be via injection into a tumor site or into a tumor. Regimens of administration can be readily determined based on factors such as exact nature and type of the tumor or cancer being treated, the severity of the tumor or cancer, the age and general physical condition of the subject, body weight of the subject, response of the individual subject, and the like.

The frequency of administration can depend on the half-life of the recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines in the subject, the condition of the subject, and the route of administration, among other factors. The frequency can be, for example, daily, weekly, monthly, quarterly, or at irregular intervals in response to changes in the subject's condition or progression of the tumor or cancer being treated. The course of treatment can depend on the condition of the subject and other factors. For example, the course of treatment can be several weeks, several months, or several years (e.g., up to 2 years). For example, repeat administrations (doses) may be undertaken immediately following the first course of treatment or after an interval of days, weeks or months to achieve tumor regression or suppression of tumor growth. Assessment may be determined by any known technique, including diagnostic methods such as imaging techniques, analysis of serum tumor markers, biopsy, or the presence, absence, or amelioration of tumor-associated symptoms. As a specific example, the recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines can be administered every 3 weeks for up to 2 years. In one example, a recombinant fusion polypeptide or chimeric polypeptide, a nucleic acid encoding a recombinant fusion polypeptide or chimeric polypeptide (e.g., a minigene construct), a recombinant bacteria or Listeria strain, an immunogenic composition, a pharmaceutical composition, or a vaccine disclosed herein is administered in increasing doses in order to increase the T-effector cell to regulatory T cell ratio and generate a more potent anti-tumor immune response. Anti-tumor immune responses can be further strengthened by providing the subject with cytokines including, for example, IFN-γ, TNF-α, and other cytokines known to enhance cellular immune response. See, e.g., U.S. Pat. No. 6,991,785, herein incorporated by reference in its entirety for all purposes.

Some methods may further comprise “boosting” the subject with additional recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines or administering the recombinant fusion polypeptides or chimeric polypeptides, nucleic acids encoding recombinant fusion polypeptides or chimeric polypeptides (e.g., minigene constructs), recombinant bacteria or Listeria strains, immunogenic compositions, pharmaceutical compositions, or vaccines multiple times. “Boosting” refers to administering an additional dose to a subject. For example, in some methods, 2 boosts (or a total of 3 inoculations) are administered, 3 boosts are administered, 4 boosts are administered, 5 boosts are administered, or 6 or more boosts are administered. The number of dosages administered can depend on, for example, the response of the tumor or cancer to the treatment.

Optionally, the recombinant fusion polypeptide or chimeric polypeptide, nucleic acids encoding a recombinant fusion polypeptide or chimeric polypeptide (e.g., minigene constructs), recombinant bacteria or Listeria strain, immunogenic composition, pharmaceutical composition, or vaccine used in the booster inoculation is the same as the recombinant fusion polypeptide or chimeric polypeptide, recombinant bacteria or Listeria strain, immunogenic composition, pharmaceutical composition, or vaccine used in the initial “priming” inoculation. Alternatively, the booster recombinant fusion polypeptide or chimeric polypeptide, recombinant bacteria or Listeria strain, immunogenic composition, pharmaceutical composition, or vaccine is different from the priming recombinant fusion polypeptide or chimeric polypeptide, recombinant bacteria or Listeria strain, immunogenic composition, pharmaceutical composition, or vaccine. Optionally, the same dosages are used in the priming and boosting inoculations. Alternatively, a larger dosage is used in the booster, or a smaller dosage is used in the booster. The period between priming and boosting inoculations can be experimentally determined. For example, the period between priming and boosting inoculations can be 1 week, 2 weeks, 3 weeks, 4 weeks, 5 weeks, 6-8 weeks, or 8-10 weeks.

Heterologous prime boost strategies have been effective for enhancing immune responses and protection against numerous pathogens. See, e.g., Schneider et al. (1999) Immunol. Rev. 170:29-38; Robinson (2002) Nat. Rev. Immunol. 2:239-250; Gonzalo et al. (2002) Vaccine 20:1226-1231; and Tanghe (2001) Infect. Immun. 69:3041-3047, each of which is herein incorporated by reference in its entirety for all purposes. Providing antigen in different forms in the prime and the boost injections can maximize the immune response to the antigen. DNA vaccine priming followed by boosting with protein in adjuvant or by viral vector delivery of DNA encoding antigen is one effective way of improving antigen-specific antibody and CD4+ T-cell responses or CD8+ T-cell responses. See, e.g., Shiver et al. (2002) Nature 415: 331-335; Gilbert et al. (2002) Vaccine 20:1039-1045; Billaut-Mulot et al. (2000) Vaccine 19:95-102; and Sin et al. (1999) DNA Cell Biol. 18:771-779, each of which is herein incorporated by reference in its entirety for all purposes. As one example, adding CRL1005 poloxamer (12 kDa, 5% POE) to DNA encoding an antigen can enhance T-cell responses when subjects are vaccinated with a DNA prime followed by a boost with an adenoviral vector expressing the antigen. See, e.g., Shiver et al. (2002) Nature 415:331-335, herein incorporated by reference in its entirety for all purposes. As another example, a vector construct encoding an immunogenic portion of an antigen and a protein comprising the immunogenic portion of the antigen can be administered. See, e.g., US 2002/0165172, herein incorporated by reference in its entirety for all purposes. Similarly, an immune response of nucleic acid vaccination can be enhanced by simultaneous administration of (e.g., during the same immune response, preferably within 0-10 or 3-7 days of each other) a polynucleotide and polypeptide of interest. See, e.g., U.S. Pat. No. 6,500,432, herein incorporated by reference in its entirety for all purposes.

The therapeutic methods disclosed herein can also comprise administering one or more additional compounds effective in preventing or treating cancer. For example, an additional compound may comprise a compound useful in chemotherapy, such as amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil (5-FU), gemcitabine, gliadelimplants, hydroxycarbamide, idarubicin, ifosfamide, irinotecan, leucovorin, liposomaldoxorubicin, liposomaldaunorubicin, lomustine, melphalan, mercaptopurine, mesna, methotrexate, mitomycin, mitoxantrone, oxaliplatin, paclitaxel (Taxol), pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Alternatively, an additional compound can also comprise other biologics, including Herceptin® (trastuzumab) against the HER2 antigen, Avastin® (bevacizumab) against VEGF, or antibodies to the EGF receptor, such as Erbitux® (cetuximab), and Vectibix® (panitumumab). Alternatively, an additional compound can comprise other immunotherapies. Alternatively, the additional compound can be an indoleamine 2,3-dioxygenase (IDO) pathway inhibitor, such as 1-methyltryptophan (1MT), 1-methyltryptophan (1MT), Necrostatin-1, Pyridoxal Isonicotinoyl Hydrazone, Ebselen, 5-Methylindole-3-carboxaldehyde, CAY10581, an anti-IDO antibody, or a small molecule IDO inhibitor. IDO inhibition can enhance the efficacy of chemotherapeutic agents. The therapeutic methods disclosed herein can also be combined with radiation, stem cell treatment, surgery, or any other treatment.

Such additional compounds or treatments can precede the administration of a recombinant fusion polypeptide or chimeric polypeptide, a nucleic acid encoding a recombinant fusion polypeptide or chimeric polypeptide (e.g., minigene construct), a recombinant bacteria or Listeria strain, an immunogenic composition, a pharmaceutical composition, or a vaccine disclosed herein, follow the administration of a recombinant fusion polypeptide or chimeric polypeptide, a nucleic acid encoding a recombinant fusion polypeptide or chimeric polypeptide (e.g., minigene construct), a recombinant bacteria or Listeria strain, an immunogenic composition, a pharmaceutical composition, or a vaccine disclosed herein, or be simultaneous to the administration of a recombinant fusion polypeptide or chimeric polypeptide, a nucleic acid encoding a recombinant fusion polypeptide or chimeric polypeptide (e.g., minigene construct), a recombinant bacteria or Listeria strain, an immunogenic composition, a pharmaceutical composition, or a vaccine disclosed herein.

Targeted immunomodulatory therapy is focused primarily on the activation of costimulatory receptors, for example by using agonist antibodies that target members of the tumor necrosis factor receptor superfamily, including 4-1BB, OX40, and GITR (glucocorticoid-induced TNF receptor-related). The modulation of GITR has demonstrated potential in both antitumor and vaccine settings. Another target for agonist antibodies are co-stimulatory signal molecules for T cell activation. Targeting costimulatory signal molecules may lead to enhanced activation of T cells and facilitation of a more potent immune response. Co-stimulation may also help prevent inhibitory influences from checkpoint inhibition and increase antigen-specific T cell proliferation.

Listeria-based immunotherapy acts by inducing the de novo generation of tumor antigen-specific T cells that infiltrate and destroy the tumor and by reducing the numbers and activities of immunosuppressive regulatory T cells (Tregs) and myeloid-derived suppressor cells (MDSCs) in the tumor microenvironment. Antibodies (or functional fragments thereof) for T cell co-inhibitory or co-stimulatory receptors (e.g., checkpoint inhibitors CTLA-4, PD-1, TIM-3, LAG3 and co-stimulators CD137, OX40, GITR, and CD40) can have synergy with Listeria-based immunotherapy.

Thus, some methods can comprise further administering a composition comprising an immune checkpoint inhibitor antagonist, such as a PD-1 signaling pathway inhibitor, a CD-80/86 and CTLA-4 signaling pathway inhibitor, a T cell membrane protein 3 (TIM3) signaling pathway inhibitor, an adenosine A2a receptor (A2aR) signaling pathway inhibitor, a lymphocyte activation gene 3 (LAG3) signaling pathway inhibitor, a killer immunoglobulin receptor (KIR) signaling pathway inhibitor, a CD40 signaling pathway inhibitor, or any other antigen-presenting cell/T cell signaling pathway inhibitor. Examples of immune checkpoint inhibitor antagonists include an anti-PD-L1/PD-L2 antibody or fragment thereof, an anti-PD-1 antibody or fragment thereof, an anti-CTLA-4 antibody or fragment thereof, or an anti-B7-H4 antibody or fragment thereof. For example, an anti PD-1 antibody can be administered to a subject at 5-10 mg/kg every 2 weeks, 5-10 mg/kg every 3 weeks, 1-2 mg/kg every 3 weeks, 1-10 mg/kg every week, 1-10 mg/kg every 2 weeks, 1-10 mg/kg every 3 weeks, or 1-10 mg/kg every 4 weeks.

Likewise, some methods can further comprise administering a T cell stimulator, such as an antibody or functional fragment thereof binding to a T-cell receptor co-stimulatory molecule, an antigen presenting cell receptor binding co-stimulatory molecule, or a member of the TNF receptor superfamily. The T-cell receptor co-stimulatory molecule can comprise, for example, CD28 or ICOS. The antigen presenting cell receptor binding co-stimulatory molecule can comprise, for example, a CD80 receptor, a CD86 receptor, or a CD46 receptor. The TNF receptor superfamily member can comprise, for example, glucocorticoid-induced TNF receptor (GITR), OX40 (CD134 receptor), 4-1BB (CD137 receptor), or TNFR25.

For example, some methods can further comprise administering an effective amount of a composition comprising an antibody or functional fragment thereof binding to a T-cell receptor co-stimulatory molecule or an antibody or functional fragment thereof binding to an antigen presenting cell receptor binding a co-stimulatory molecule. The antibody can be, for example, an anti-TNF receptor antibody or antigen-binding fragment thereof (e.g., TNF receptor superfamily member glucocorticoid-induced TNF receptor (GITR), OX40 (CD134 receptor), 4-1BB (CD137 receptor), or TNFR25), an anti-OX40 antibody or antigen-binding fragment thereof, or an anti-GITR antibody or antigen binding fragment thereof. Alternatively, other agonistic molecules can be administered (e.g., GITRL, an active fragment of GITRL, a fusion protein containing GITRL, a fusion protein containing an active fragment of GITRL, an antigen presenting cell (APC)/T cell agonist, CD134 or a ligand or fragment thereof, CD137 or a ligand or fragment thereof, or an inducible T cell costimulatory (ICOS) or a ligand or fragment thereof, or an agonistic small molecule).

In a specific example, some methods can further comprise administering an anti-CTLA-4 antibody or a functional fragment thereof and/or an anti-CD137 antibody or functional fragment thereof. For example, the anti-CTLA-4 antibody or a functional fragment thereof or the anti-CD137 antibody or functional fragment thereof can be administered about 72 hours after the first dose of recombinant fusion polypeptide or chimeric polypeptide, nucleic acids encoding a recombinant fusion polypeptide or chimeric polypeptide (e.g., minigene constructs), recombinant bacteria or Listeria strain, immunogenic composition, pharmaceutical composition, or vaccine, or about 48 hours after the first dose of recombinant fusion polypeptide or chimeric polypeptide, nucleic acids encoding a recombinant fusion polypeptide or chimeric polypeptide (e.g., minigene constructs), recombinant bacteria or Listeria strain, immunogenic composition, pharmaceutical composition, or vaccine. The anti-CTLA-4 antibody or a functional fragment thereof or anti-CD137 antibody or functional fragment thereof can be administered at a dose, for example, of about 0.05 mg/kg and about 5 mg/kg. A recombinant Listeria strain or immunogenic composition comprising a recombinant Listeria strain can be administered at a dose, for example, of about 1×10⁹ CFU. Some such methods can further comprise administering an effective amount of an anti-PD-1 antibody or functional fragment thereof.

Methods for assessing efficacy of cancer immunotherapies are well known and are described, for example, in Dzojic et al. (2006) Prostate 66(8):831-838; Naruishi et al. (2006) Cancer Gene Ther. 13(7):658-663, Sehgal et al. (2006) Cancer Cell Int. 6:21), and Heinrich et al. (2007) Cancer Immunol Immunother 56(5):725-730, each of which is herein incorporated by reference in its entirety for all purposes. As one example, for prostate cancer, a prostate cancer model can be to test methods and compositions disclosed herein, such as a TRAMP-C2 mouse model, a 178-2 BMA cell model, a PAIII adenocarcinoma cells model, a PC-3M model, or any other prostate cancer model.

Alternatively or additionally, the immunotherapy can be tested in human subjects, and efficacy can be monitored using known. Such methods can include, for example, directly measuring CD4+ and CD8+ T cell responses, or measuring disease progression (e.g., by determining the number or size of tumor metastases, or monitoring disease symptoms such as cough, chest pain, weight loss, and so forth). Methods for assessing the efficacy of a cancer immunotherapy in human subjects are well known and are described, for example, in Uenaka et al. (2007) Cancer Immun. 7:9 and Thomas-Kaskel et al. (2006) Int J Cancer 119(10):2428-2434, each of which is herein incorporated by reference in its entirety for all purposes.

### VII. Kits

Also provided are kits comprising a reagent utilized in performing a method disclosed herein or kits comprising a composition, tool, or instrument disclosed herein.

For example, such kits can comprise a recombinant fusion polypeptide or chimeric polypeptide disclosed herein, a nucleic acid encoding a recombinant fusion polypeptide or chimeric polypeptide disclosed herein (e.g., a minigene nucleic acid construct disclosed herein), a recombinant bacteria or Listeria strain disclosed herein, an immunogenic composition disclosed herein, a pharmaceutical composition disclosed herein, or a vaccine disclosed herein. Such kits can additionally comprise an instructional material which describes use of the recombinant fusion polypeptide or chimeric polypeptide, the nucleic acid encoding the recombinant fusion polypeptide or chimeric polypeptide (e.g., a minigene nucleic acid construct), the recombinant Listeria strain, the immunogenic composition, the pharmaceutical composition, or the vaccine to perform the methods disclosed herein. Such kits can optionally further comprise an applicator. Although model kits are described below, the contents of other useful kits will be apparent in light of the present disclosure.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Listing of Embodiments

The subject matter disclosed herein includes, but is not limited to, the following embodiments.

1. An immunogenic composition comprising a recombinant Listeria strain comprising a recombinant attenuated Listeria strain comprising a nucleic acid encoding a recombinant polypeptide, wherein the recombinant polypeptide comprises a truncated listeriolysin O (LLO) protein, a truncated ActA protein, or a PEST amino acid sequence fused to a Wilms tumor protein or an immunogenic fragment thereof.

2. The composition of embodiment 1, wherein the recombinant polypeptide comprises a truncated listeriolysin O (LLO) protein, a truncated ActA protein, or a PEST amino acid sequence fused to a full-length Wilms tumor protein.

3. The composition of embodiment 1, wherein the recombinant polypeptide comprises a truncated listeriolysin O (LLO) protein, a truncated ActA protein, or a PEST amino acid sequence fused to the Wilms tumor protein immunogenic fragment set forth in SEQ ID NO: 95.

4. The composition of any preceding embodiment, wherein the Wilms tumor protein or the immunogenic fragment thereof is fused to a truncated listeriolysin O (tLLO) protein.

5. The composition of embodiment 4, wherein the tLLO comprises, consists essentially of, or consists of the sequence set forth in SEQ ID NO: 57.

6. The composition of any preceding embodiment, wherein the Listeria strain is a Listeria monocytogenes strain.

7. The composition of embodiment 6, wherein the Listeria monocytogenes strain is a Listeria monocytogenes dal(−)dat(−)ΔactA (LmddA) strain.

8. A method of treating a cancer in a subject, comprising administering the composition of any one of embodiments 1-7 to the subject.

9. A method of eliciting an enhanced anti-cancer T cell response in a subject, comprising administering the composition of any one of embodiments 1-7 to the subject.

10. A method of eliciting an enhanced immune response against a cancer in a subject, comprising administering the composition of any one of embodiments 1-7 to the subject.

11. The method of any one of embodiments 8-10, wherein the cancer is a breast cancer.

12. The method of embodiment 11, wherein the breast cancer is triple negative breast cancer.

13. The method of any one of embodiments 8-10, wherein the cancer is a gastrointestinal cancer.

The subject matter disclosed herein also includes, but is not limited to, the following embodiments.

1. A minigene construct comprising a nucleic acid comprising an open reading frame encoding a chimeric polypeptide, wherein the chimeric polypeptide comprises, consists essentially of, or consists of: (a) a bacterial secretion signal sequence; (b) a ubiquitin protein; and (c) an antigenic Wilms tumor protein (WT1) peptide, wherein the bacterial secretion signal sequence, the ubiquitin, and the antigenic WT1 peptide are arranged in tandem from the amino-terminal end to the carboxy-terminal end of the chimeric polypeptide.

2. The minigene construct of embodiment 1, wherein the antigenic WT1 peptide comprises a heteroclitic mutant WT1 peptide.

3. The minigene construct of embodiment 2, wherein the heteroclitic mutant WT1 peptide comprises, consists essentially of, or consists of the sequence set forth in any one of SEQ ID NOS: 114-137, 159, 160, 162, and 163.

4. The minigene construct of embodiment 3, wherein the heteroclitic mutant WT1 peptide comprises, consists essentially of, or consists of the sequence set forth in SEQ ID NO: 114, 115, 162, or 163.

5. The minigene construct of embodiment 4, wherein the chimeric polypeptide comprise, consists essentially of, or consists of s the sequence set forth in SEQ ID NO: 156, 157, 165, or 166.

6. The minigene construct of any embodiment 1, wherein the antigenic WT1 peptide comprises a native WT1 peptide.

7. The minigene construct of embodiment 6, wherein the native WT1 peptide comprises, consists essentially of, or consists of the sequence set forth in SEQ ID NO: 95 or 158.

8. The minigene construct of embodiment 7, wherein the chimeric polypeptide comprises, consists essentially of, or consists of the sequence set forth in SEQ ID NO: 164.

9. The minigene construct of any one of embodiments 1-4, 6, and 7, wherein the chimeric polypeptide further comprises one or more additional antigenic WT1 peptides between the bacterial signal sequence and the ubiquitin protein.

10. The minigene construct of embodiment 9, wherein the one or more additional antigenic WT1 peptides comprise two or more additional antigenic WT1 peptides.

11. The minigene construct of embodiment 10, wherein the two or more additional antigenic WT1 peptides are fused directly to each other without intervening sequence.

12. The minigene construct of embodiment 10, wherein the two or more additional antigenic WT1 peptides are linked to each other via peptide linkers.

13. The minigene construct of any one of embodiments 9-12, wherein the one or more additional antigenic peptides comprise, consist essentially of, or consist of a first additional antigenic WT1 peptide comprising, consisting essentially of, or consisting of the sequence set forth in SEQ ID NO: 154, a second additional antigenic WT1 peptide comprising, consisting essentially of, or consisting of the sequence set forth in SEQ ID NO: 155, and a third additional antigenic WT1 peptide comprising, consisting essentially of, or consisting of the sequence set forth in SEQ ID NO: 136.

14. The minigene construct of any one of embodiments 9-12, wherein the one or more additional antigenic peptides comprise, consist essentially of, or consist of a first additional antigenic WT1 peptide comprising, consisting essentially of, or consisting of the sequence set forth in SEQ ID NO: 154, a second additional antigenic WT1 peptide comprising, consisting essentially of, or consisting of the sequence set forth in SEQ ID NO: 155, and a third additional antigenic WT1 peptide comprising, consisting essentially of, or consisting of the sequence set forth in SEQ ID NO: 137.

15. The minigene construct of embodiment 14, wherein the chimeric polypeptide comprises, consists essentially of, or consists of the sequence set forth in SEQ ID NO: 153.

16. The minigene construct of any preceding embodiment, wherein the bacterial secretion signal sequence is a listeriolysin O (LLO) secretion signal sequence.

17. The minigene construct of embodiment 16, wherein the LLO secretion signal sequence comprises, consists essentially of, or consists of the sequence set forth in SEQ ID NO: 59 or SEQ ID NO: 150.

18. The minigene construct of any preceding embodiment, wherein the ubiquitin protein comprises, consists essentially of, or consists of the sequence set forth in SEQ ID NO: 100.

19. The minigene construct of any preceding embodiment, wherein the antigenic WT1 peptide comprises, consists essentially of, or consists of the sequence set forth in any one of SEQ ID NOS: 95, 114-137, 158, 162, and 163, the bacterial secretion signal sequences comprises, consists essentially of, or consists of the sequence set forth in SEQ ID NO: 59, and the ubiquitin protein comprises, consists essentially of, or consists of the sequence set forth in SEQ ID NO: 100.

20. The minigene construct of any preceding embodiment, wherein the minigene construct comprises two or more open reading frames, each encoding a chimeric polypeptide comprising, consisting essentially of, or consisting of: (a) a bacterial secretion signal sequence; (b) a ubiquitin protein; and (c) an antigenic WT1 peptide, wherein the bacterial secretion signal sequence, the ubiquitin, and the antigenic WT1 peptide are arranged in tandem from the amino-terminal end to the carboxy-terminal end of each chimeric polypeptide, and wherein the antigenic WT1 peptide is different in each chimeric polypeptide, and wherein the minigene construct comprises a Shine-Dalgarno ribosome binding site nucleic acid sequence between each pair of open reading frames.

21. A chimeric polypeptide encoded by the minigene construct of any preceding embodiment.

22. A recombinant Listeria strain comprising the minigene construct of any one of embodiments 1-20.

23. A recombinant Listeria strain comprising a nucleic acid comprising a first open reading frame encoding a fusion polypeptide, wherein the fusion polypeptide comprises a PEST-containing peptide fused to an antigenic peptide selected from a native Wilms tumor protein (WT1), an immunogenic fragment of the native WT1, and a WT1 peptide comprising, consisting essentially of, or consisting of the sequence set forth in SEQ ID NO: 114 or SEQ ID NO: 136.

24. The recombinant Listeria strain of embodiment 23, wherein the fusion polypeptide comprises the native WT1.

25. The recombinant Listeria strain of embodiment 23, wherein the fusion polypeptide comprises the immunogenic fragment of the native WT1.

26. The recombinant Listeria strain of embodiment 25, wherein the immunogenic fragment of the native WT1 comprises, consists essentially of, or consists of the sequence set forth in SEQ ID NO: 95 or SEQ ID NO: 139.

27. The recombinant Listeria strain of embodiment 23, wherein the fusion polypeptide comprises the WT1 peptide comprising, consisting essentially of, or consisting of the sequence set forth in SEQ ID NO: 114 or SEQ ID NO: 136.

28. The recombinant Listeria strain of any one of embodiments 23-27, wherein the fusion polypeptide comprises the PEST-containing peptide fused to two or more antigenic WT1 peptides, wherein at least one of the two or more antigenic WT1 peptides is selected from the native Wilms tumor protein (WT1), the immunogenic fragment of the native WT1, and the WT1 peptide comprising, consisting essentially of, or consisting of the sequence set forth in SEQ ID NO: 114 or SEQ ID NO: 136, and wherein the two or more antigenic WT1 peptides are the same or different.

29. The recombinant Listeria strain of any one of embodiments 23-28, wherein the two or more antigenic WT1 peptides are fused directly to each other without intervening sequence.

30. The recombinant Listeria strain of any one of embodiments 23-28, wherein the two or more antigenic WT1 peptides are linked to each other via peptide linkers.

31. The recombinant Listeria strain of any one of embodiments 23-30, wherein the fusion polypeptide further comprises one or more peptide tags N-terminal and/or C-terminal to the one or more of the native Wilms tumor protein (WT1), the immunogenic fragment of the native WT1, and the WT1 peptide comprising, consisting essentially of, or consisting of the sequence set forth in SEQ ID NO: 114 or SEQ ID NO: 136.

32. The recombinant Listeria strain of embodiment 31, wherein the one or more peptide tags comprise one or more of the following: 3×FLAG tag; 6×His tag; SIINFEKL tag, and the FLAG tag set forth in SEQ ID NO: 99.

33. The recombinant Listeria strain of any one of embodiments 23-32, wherein the PEST-containing peptide is on the N-terminal end of the fusion polypeptide.

34. The recombinant Listeria strain of any one of embodiments 23-33, wherein the PEST-containing peptide is an N-terminal fragment of LLO.

35. The recombinant Listeria strain of embodiment 34, wherein the N-terminal fragment of LLO comprises, consists essentially of, or consists of the sequence set forth in SEQ ID NO: 59.

36. The recombinant Listeria strain of any one of embodiments 22-35, wherein the nucleic acid is operably integrated into the Listeria genome.

37. The recombinant Listeria strain of any one of embodiments 22-35, wherein the nucleic acid is in an episomal plasmid.

38. The recombinant Listeria strain of any one of embodiments 22-37, wherein the nucleic acid does not confer antibiotic resistance upon the recombinant Listeria strain.

39. The recombinant Listeria strain of any one of embodiments 22-38, wherein the recombinant Listeria strain is an attenuated, auxotrophic Listeria strain.

40. The recombinant Listeria strain of embodiment 39, wherein the attenuated Listeria strain comprises a mutation in one or more endogenous genes that inactivates the one or more endogenous genes.

41. The recombinant Listeria strain of embodiment 40, wherein the one or more endogenous genes comprise prfA.

42. The recombinant Listeria strain of embodiment 40, wherein the one or more endogenous genes comprise actA.

43. The recombinant Listeria strain of embodiment 40, wherein the one or more endogenous genes comprise actA and inlB.

44. The recombinant Listeria strain of embodiment 40, wherein the one or more endogenous genes comprise actA, dal, and dat.

45. The recombinant Listeria strain of any one of embodiments 22-44, wherein the nucleic acid comprises a second open reading frame encoding a metabolic enzyme.

46. The recombinant Listeria strain of embodiment 45, wherein the metabolic enzyme is an alanine racemase enzyme or a D-amino acid aminotransferase enzyme.

47. The recombinant Listeria strain of any one of embodiments 22-46, wherein the fusion polypeptide is expressed from an hly promoter.

48. The recombinant Listeria strain of any one of embodiments 22-47, wherein the recombinant Listeria strain is a recombinant Listeria monocytogenes strain.

49. The recombinant Listeria strain of any one of embodiments 22-33, wherein the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein.

50. The recombinant Listeria strain of any one of embodiments 22-33, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of LLO.

51. The recombinant Listeria strain of any one of embodiments 22-33, wherein recombinant Listeria strain is an attenuated Listeria monocytogenes strain comprising a deletion of or inactivating mutation in actA and inlB, wherein the nucleic acid is genomically integrated, and wherein the PEST-containing peptide is an ActA protein or a fragment thereof.

52. An immunogenic composition comprising the recombinant Listeria strain of any one of embodiments 22-51 and an adjuvant.

53. The immunogenic composition of embodiment 52, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

54. A method of inducing an immune response against a WT1-expressing tumor or cancer in a subject, comprising administering to the subject the recombinant Listeria strain of any one of embodiments 22-51 or the immunogenic composition of any one of embodiments 52-53.

55. A method of preventing or treating a WT1-expressing tumor or cancer in a subject, comprising administering to the subject the recombinant Listeria strain of any one of embodiments 22-51 or the immunogenic composition of any one of embodiments 52-53.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

| SEQ | | |
|---|---|---|
| ID NO | Type | Description |
| 1 | DNA | SIINFEKL Tag v1 |
| 2 | DNA | SIINFEKL Tag v2 |
| 3 | DNA | SIINFEKL Tag v3 |
| 4 | DNA | SIINFEKL Tag v4 |
| 5 | DNA | SIINFEKL Tag v5 |
| 6 | DNA | SIINFEKL Tag v6 |
| 7 | DNA | SIINFEKL Tag v7 |
| 8 | DNA | SIINFEKL Tag v8 |
| 9 | DNA | SIINFEKL Tag v9 |
| 10 | DNA | SIINFEKL Tag v10 |
| 11 | DNA | SIINFEKL Tag v11 |
| 12 | DNA | SIINFEKL Tag v12 |
| 13 | DNA | SIINFEKL Tag v13 |
| 14 | DNA | SIINFEKL Tag v14 |
| 15 | DNA | SIINFEKL Tag v15 |
| 16 | Protein | SIINFEKL Tag |
| 17 | DNA | 3xFLAG Tag v1 |
| 18 | DNA | 3xFLAG Tag v2 |
| 19 | DNA | 3xFLAG Tag v3 |
| 20 | DNA | 3xFLAG Tag v4 |
| 21 | DNA | 3xFLAG Tag v5 |
| 22 | DNA | 3xFLAG Tag v6 |
| 23 | DNA | 3xFLAG Tag v7 |
| 24 | DNA | 3xFLAG Tag v8 |
| 25 | DNA | 3xFLAG Tag v9 |
| 26 | DNA | 3xFLAG Tag v10 |
| 27 | DNA | 3xFLAG Tag v11 |
| 28 | DNA | 3xFLAG Tag v12 |
| 29 | DNA | 3xFLAG Tag v13 |
| 30 | DNA | 3xFLAG Tag v14 |
| 31 | DNA | 3xFLAG Tag v15 |
| 32 | Protein | 3xFLAG Tag |
| 33 | Protein | Peptide Linker v1 |
| 34 | Protein | Peptide Linker v2 |
| 35 | Protein | Peptide Linker v3 |
| 36 | Protein | Peptide Linker v4 |
| 37 | Protein | Peptide Linker v5 |
| 38 | Protein | Peptide Linker v6 |
| 39 | Protein | Peptide Linker v7 |
| 40 | Protein | Peptide Linker v8 |
| 41 | Protein | Peptide Linker v9 |
| 42 | Protein | Peptide Linker v10 |
| 43 | Protein | PEST-Like Sequence v1 |
| 44 | Protein | PEST-Like Sequence v2 |
| 45 | Protein | PEST-Like Sequence v3 |
| 46 | Protein | PEST-Like Sequence v4 |
| 47 | Protein | PEST-Like Sequence v5 |
| 48 | Protein | PEST-Like Sequence v6 |
| 49 | Protein | PEST-Like Sequence v7 |
| 50 | Protein | PEST-Like Sequence v8 |
| 51 | Protein | PEST-Like Sequence v9 |
| 52 | Protein | PEST-Like Sequence v10 |
| 53 | Protein | PEST-Like Sequence v11 |
| 54 | Protein | PEST-Like Sequence v12 |
| 55 | Protein | LLO Protein v1 |
| 56 | Protein | LLO Protein v2 |
| 57 | Protein | N-Terminal Truncated LLO v1 |
| 58 | Protein | N-Terminal Truncated LLO v2 |
| 59 | Protein | N-Terminal Truncated LLO v3 |
| 60 | DNA | Nucleic Acid Encoding N-Terminal |
| | | Truncated LLO v3 |
| 61 | Protein | ActA Protein v1 |
| 62 | Protein | ActA Protein v2 |
| 63 | Protein | ActA Fragment v1 |
| 64 | Protein | ActA Fragment v2 |
| 65 | Protein | ActA Fragment v3 |
| 66 | Protein | ActA Fragment v4 |
| 67 | Protein | ActA Fragment v5 |
| 68 | DNA | Nucleic Acid Encoding ActA |
| | | Fragment v5 |
| 69 | Protein | ActA Fragment v6 |
| 70 | Protein | ActA Fragment v7 |
| 71 | DNA | Nucleic Acid Encoding ActA |
| | | Fragment v7 |
| 72 | Protein | ActA Fragment Fused to Hly Signal |
| | | Peptide |
| 73 | Protein | ActA Substitution |
| 74 | Protein | Cholesterol-Binding Domain of LLO |
| 75 | Protein | HLA-A2 restricted Epitope from |
| | | NY-ESO-1 |
| 76 | Protein | Lm Alanine Racemase |
| 77 | Protein | Lm D-Amino Acid Aminotransferase |
| 78 | DNA | Nucleic Acid Encoding Lm Alanine |
| | | Racemase |
| 79 | DNA | Nucleic Acid Encoding Lm D-Amino |
| | | Acid Aminotransferase |
| 80 | Protein | Wild Type PrfA |
| 81 | DNA | Nucleic Acid Encoding Wild Type |
| | | PrfA |
| 82 | Protein | D133V PrfA |
| 83 | DNA | Nucleic Acid Encoding D133V PrfA |
| 84 | DNA | 4X Glycine Linker G1 |
| 85 | DNA | 4X Glycine Linker G2 |
| 86 | DNA | 4X Glycine Linker G3 |
| 87 | DNA | 4X Glycine Linker G4 |
| 88 | DNA | 4X Glycine Linker G5 |
| 89 | DNA | 4X Glycine Linker G6 |
| 90 | DNA | 4X Glycine Linker G7 |
| 91 | DNA | 4X Glycine Linker G8 |
| 92 | DNA | 4X Glycine Linker G9 |
| 93 | DNA | 4X Glycine Linker G10 |
| 94 | DNA | 4X Glycine Linker G11 |
| 95 | Protein | Wilms Tumor Protein Fragment |
| 96 | Protein | C-terminal SIINFEKL-S-6xHIS tag |
| 97 | Protein | WT1 Single Construct |
| 98 | Protein | SIINFEKL |
| 99 | Protein | FLAG Tag |
| 100 | Protein | Ubiquitin |
| 101 | Protein | Wild-Type WT1 Peptide v1 (A1) |
| 102 | Protein | Wild-Type WT1 Peptide v2 |
| 103 | Protein | Wild-Type WT1 Peptide v3 |
| 104 | Protein | Wild-Type WT1 Peptide v4 |
| 105 | Protein | Wild-Type WT1 Peptide v5 |
| 106 | Protein | Wild-Type WT1 Peptide v6 |
| 107 | Protein | Wild-Type WT1 Peptide v7 |
| 108 | Protein | Wild-Type WT1 Peptide v8 |
| 109 | Protein | Wild-Type WT1 Peptide v9 |
| 110 | Protein | Wild-Type WT1 Peptide v10 |
| 111 | Protein | Wild-Type WT1 Peptide v11 |
| 112 | Protein | Wild-Type WT1 Peptide v12 |
| 113 | Protein | Wild-Type WT1 Peptide v13 |
| 114 | Protein | Heteroclitic WT1 Peptide v1A |
| | | (WT1-F) |
| 115 | Protein | Heteroclitic WT1 Peptide v1B |
| | | (WT1-A1) |
| 116 | Protein | Heteroclitic WT1 Peptide v2 |
| 117 | Protein | Heteroclitic WT1 Peptide v3 |
| 118 | Protein | Heteroclitic WT1 Peptide v4 |
| 119 | Protein | Heteroclitic WT1 Peptide v5 |
| 120 | Protein | Heteroclitic WT1 Peptide v6 |
| 121 | Protein | Heteroclitic WT1 Peptide v7 |
| 122 | Protein | Heteroclitic WT1 Peptide v8 |
| 123 | Protein | Heteroclitic WT1 Peptide v9 |
| 124 | Protein | Heteroclitic WT1 Peptide v10A |
| 125 | Protein | Heteroclitic WT1 Peptide v10B |
| 126 | Protein | Heteroclitic WT1 Peptide v10C |
| 127 | Protein | Heteroclitic WT1 Peptide v11A |
| 128 | Protein | Heteroclitic WT1 Peptide v11B |
| 129 | Protein | Heteroclitic WT1 Peptide v11C |
| 130 | Protein | Heteroclitic WT1 Peptide v12A |
| 131 | Protein | Heteroclitic WT1 Peptide v12B |
| 132 | Protein | Heteroclitic WT1 Peptide v12C |
| 133 | Protein | Heteroclitic WT1 Peptide v13A |
| 134 | Protein | Heteroclitic WT1 Peptide v13B |
| 135 | Protein | Heteroclitic WT1 Peptide v13C |
| 136 | Protein | Heteroclitic WT1 Peptide v1C |
| | | (WT1-122F long) |
| 137 | Protein | Heteroclitic WT1 Peptide v1D |
| | | (WT1-122A1 long) |
| 138 | Protein | WT1-UniProt P19544 |
| 139 | Protein | WT1-UniProt P19544 Del |
| 140 | Protein | WT1-NP_001185481.1 |
| 141 | Protein | WT1-NP_001185480.1 |
| 142 | Protein | WT1-NP_077744.3 |
| 143 | Protein | WT1-NP_077742.2 |
| 144 | Protein | WT1-NP_000369.3 |
| 145 | Protein | WT1 v1 |
| 146 | Protein | WT1 v2 |
| 147 | Protein | WT1 v3 |
| 148 | Protein | WT1 v4 |
| 149 | Protein | Detoxified Listeriolysin O |
| | | (dtLLO) |
| 150 | Protein | LLO Signal Sequence |
| 151 | Protein | ActA Signal Sequence |
| 152 | Protein | Wild-Type WT1 Peptide v1B |
| 153 | Protein | WT1-tLLO-P1-P2-P3-FLAG-Ub- |
| | | Heteroclitic Tyrosine Minigene |
| | | Construct |
| 154 | Protein | Wild-Type WT1 Peptide v14 |
| | | (WT1-427 long) |
| 155 | Protein | Wild-Type WT1 Peptide v15 |
| | | (WT1-331 long) |
| 156 | Protein | WT1-tLLO-FLAG-Ub-Heteroclitic |
| | | Phenylalanine Minigene Construct |
| 157 | Protein | WT1-tLLO-FLAG-Ub-Heteroclitic |
| | | Tyrosine Minigene Construct |
| 158 | Protein | Wild-Type A24 |
| 159 | Protein | Heteroclitic A24 v1 (A24-het-1) |
| 160 | Protein | Heteroclitic A24 v2 (A24-het-2) |
| 161 | Protein | Wild-Type A24 Long |
| 162 | Protein | Heteroclitic A24 v1 Long |
| | | (A24-het-1-long) |
| 163 | Protein | Heteroclitic A24 v2 Long |
| | | (A24-het-2-long) |
| 164 | Protein | WT1-FLAG-Ub-A24 Native Minigene |
| | | Construct |
| 165 | Protein | WT1-FLAG-Ub-Heteroclitic A24 |
| | | Minigene Construct v1 |
| 166 | Protein | WT1-FLAG-Ub-Heteroclitic A24 |
| | | Minigene Construct v2 |
| 167 | DNA | Adv16 f Primer |
| 168 | DNA | Adv295 r Primer |

### EXAMPLES

### Example 1. Preparation of LmddA323 (WT1) and LmddA324 (Ova)

Sequences of WT1 and OVA were amplified from the given construct (ppl2-actA-ubiqitin-sinc pept plasmid) with new primers, purified and ligated in pCR2.1 vector. Ligation mix was transformed into Top10 E. coli cells. Colony PCR confirmed the presence of the plasmids. The plasmid sequence was verified by DNA sequencing. They were named pAdv318, pAdv319, pAdv320, pAdv321, and pAdv322.

Sequences of WT1 and OVA were ligated into pAdv134 and transformed into MB2159. Minipreps were prepared and sent for sequencing to confirm the correct ones. They were named as pAdv323, pAdv324. Plasmids pAdv323, pAdv324 were transformed in LmddA. Two passages were performed and confirmed by colony PCR for LmddA 323, LmddA 324. Sequence analysis confirmed the correct sequence for LmddA323 and LmddA324. The amino acid sequence of the WT1 peptide expressed from the minigene construct is: RMFPNAPY (SEQ ID NO: 95).

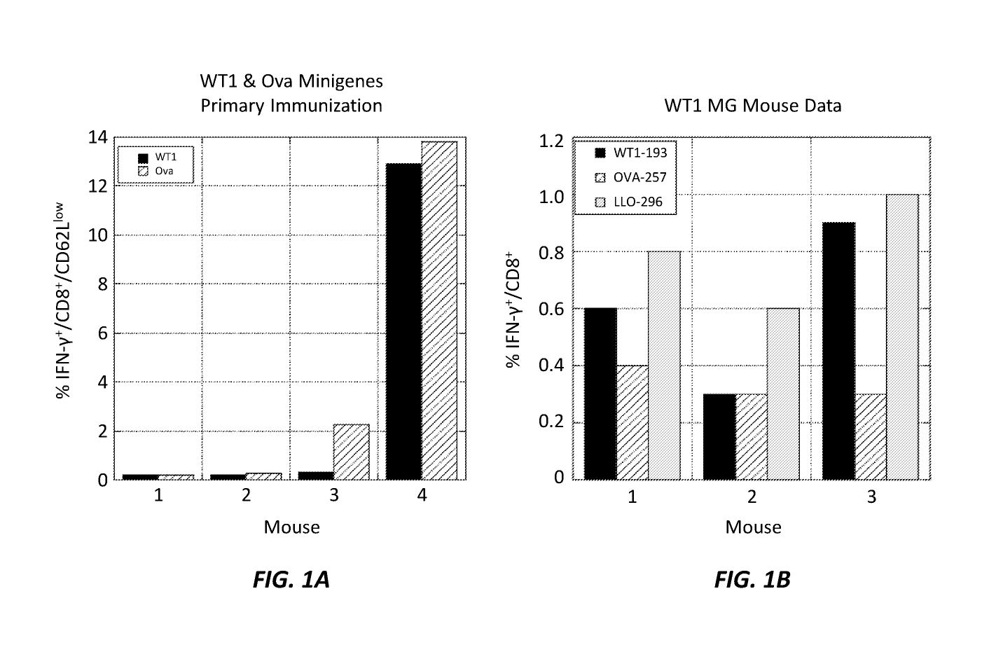

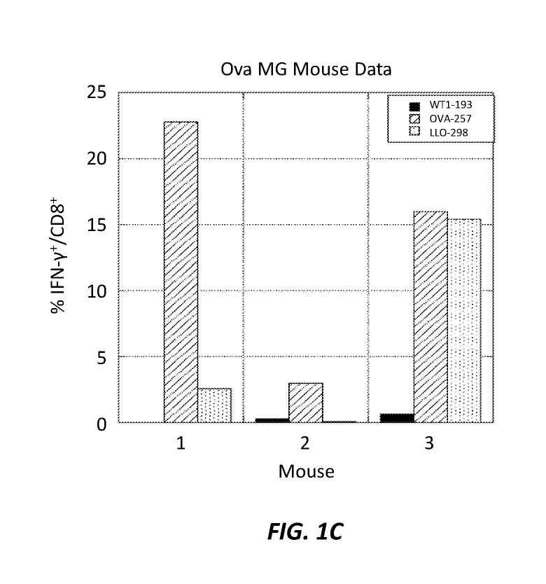

IFN-γ response to WT1 and Ova peptides in C57BL/6 mice immunized with LmddA323 (WT1) and LmddA324 (Ova) are shown in FIGS. 1A-1C. The high response to control peptide in the last mouse in FIGS. 1A and 1C is due to peptide cross contamination during in vitro stimulation. IFN-γ responses to WT1 and Ova peptides were evaluated by ICS following primary (FIG. 1A) and secondary (FIGS. 1B and 1C) immunizations. No response was detected to the WT1 peptide following primary immunization; however, 2 out of 3 mice showed a measurable response to WT1 peptide following boost immunization. Ova-specific responses were detected following both primary and boost immunization in all mice tested.

### Example 2. Generation of T Cell Lines/Clones Specific for WT1-H-2D^{b} Epitope After Immunization with Specific Listeria-Based Immunotherapies/Minigene Construct

Male and female C57BL/6 mice from Jackson Labs aged 6-8 weeks were immunized using ADXS11-001 (DP, batch—2013), ADXS31-142 (Batch,), LmddA323 (minigene—WT1), ADXS31-164 according to Table 1 below.

TABLE 1
Dates of Immunizations of mice/spleen harvest.

| | Mice/ | Dose | Dose 1 | Dose 2 | Spleens | Dose 3 | Spleens |
|---|---|---|---|---|---|---|---|
| Group | Group | Administration | 1 × 10⁸ CFU | 1 × 10⁸ CFU | Harvested | 1 × 10⁸ CFU | Harvested |
| ADXS11-001 | 4 F | IV | Apr. 8, 2015 | Apr. 22, 2015 | Apr. 28, 2015 | May 7, 2015 | May 13, 2015 |
| | | | | | (2F) | | (2F) |
| LmddA323 | 4F | IV | Apr. 8, 2015 | Apr. 22, 2015 | Apr. 28, 2015 | May 7, 2015 | May 13, 2015 |
| (minigene - | | | | | (2F) | | (2F) |
| WT1) | | | | | | | |
| ADXS31-164 | 8F | IV | Apr. 8, 2015 | Apr. 22, 2015 | Apr. 28, 2015 | May 7, 2015 | May 13, 2015 |
| | | | | | (4F) | | (4F) |
| ADXS31-142 | 4 M | IV | Apr. 8, 2015 | Apr. 22, 2015 | Apr. 28, 2015 | May 7, 2015 | May 13, 2015 |
| | | | | | (2M) | | (2M) |

The spleens from each mouse were harvested according to Table 1 above. The spleen from each mouse were collected in an individual tube containing 5 mL of c-RPMI medium. c-RPMI medium (Complete Medium) is 450 mL RPMI 1640, 50 mL FCS, 5 mL HEPES, 5 mL NEAA, 5 mL Glutamax, 5 mL Na-Pyruvate, 5 mL Pen/step, and 129 μL 2-ME (14.6M). The detailed steps for preparing isolated splenocytes are as follows.

The spleens were harvested from experimental and controls using sterile forceps and scissors and transported in 15 mL tubes containing 10 mL PBS to the lab. The spleens were then poured into a sterile Petri dish. The spleens were mashed in wash medium (RPMI only) using two glass slides or the back of plunger from a 3 mL syringe. Cells in the medium were transferred to a 15 mL tube, for 1 or 2 spleens or 50 mL tubes for more than two spleens. Cells were pelleted at 1,000 RPM for 5 min at RT. Supernatant was discarded and cells were resuspended in the remaining wash buffer gently and 2 mL RBC lysis buffer per spleen was added to the cell pellet. Cells were mixed gently with lysis buffer by tapping the tube. After waiting for 1 min, 10 mL of c-RPMI medium was immediately added to the cell suspension to deactivate lysis buffer. Cells were spun at 1,000 for 5 min at RT. The cells were passed through a cell strainer and washed one more time with 10 mL c-RPMI. Cells were counted using hemocytometer and viability was checked by Trypan blue staining. Each spleen yields approximately 1-2×10⁸ cells.

T cells specific for different antigens such as E7, PSA and WT1 are expanded in vitro after stimulation with mitomycin C treated EL4 cells pulsed with peptides, for 3-4 weeks. The expanded T cells were confirmed to be antigen-specific by pentamer staining and/or IFN-γ ICS and clones were selected by limiting dilution. The T cell clones specific for each antigen will be further used as tool for the development of an in vitro potency assay.

### Example 3. Generation of WT1 Antigen Constructs and Expression and Secretion in Antigen-Presenting Cells

Constructs were generated comprising WT1 antigens with C-terminal SIINFEKL-S-6×HIS tags (ARSIINFEKLSHHHHHH; SEQ ID NO: 96). Specifically, a construct was generated comprising a WT1 protein with a C-terminal tag. The WT1 single construct is set forth in SEQ ID NO: 97.

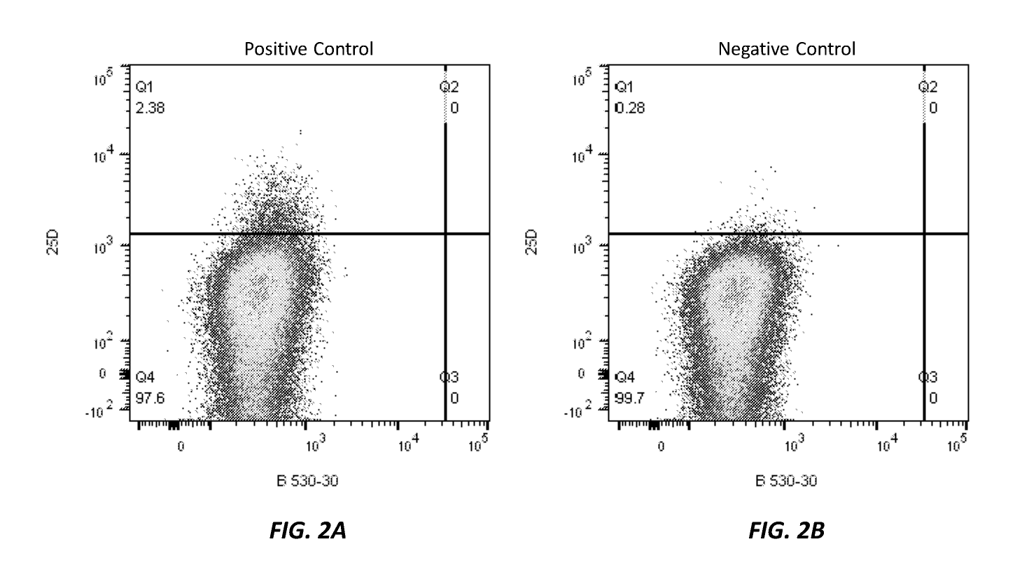

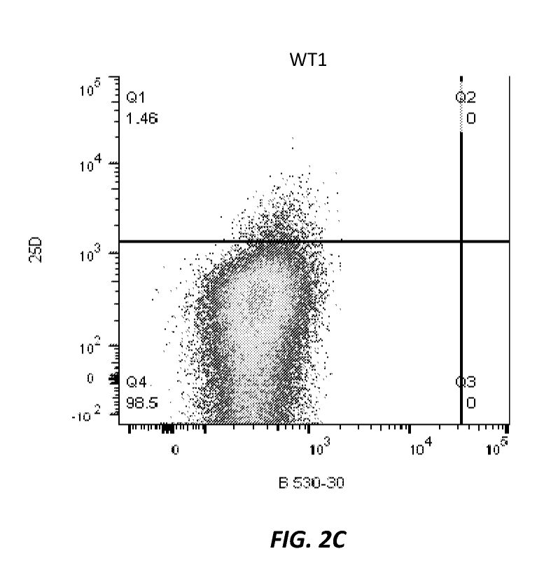

Murine dendritic DC2.4 cells were infected with Lmdda-WT1-tag expressing vector. Samples were then stained with 25D-APC conjugated antibody and run on a flow cytometer for detection of 25D-APC. Detection of the C-terminal SIINFEKL tag (SEQ ID NO: 98) with the 25D-APC conjugated antibody is shown in FIGS. 2A-2C. As shown in FIG. 2C, the WT1 sample showed high levels of positive staining comparable to the positive control in FIG. 2A and significantly higher than the negative control in FIG. 2B, confirming that the antigens express and secrete in antigen-presenting cells upon infection.

### Example 4. Evaluation of Ability of Listeria to Express Heteroclitic WT1 Minigene Fusion Proteins

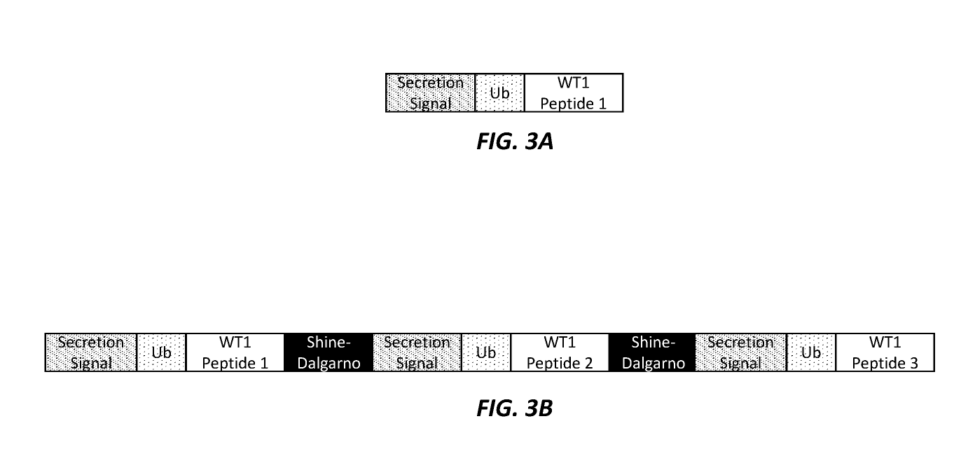

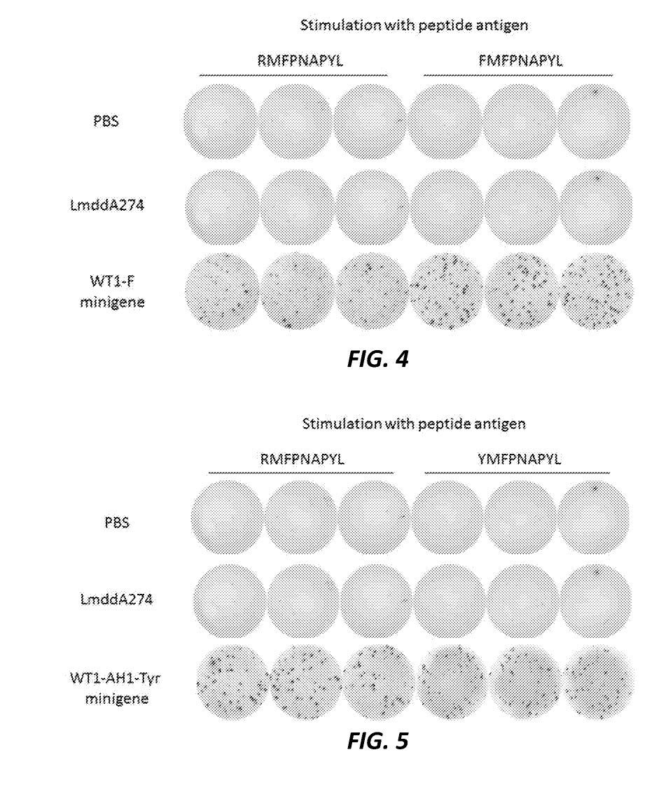

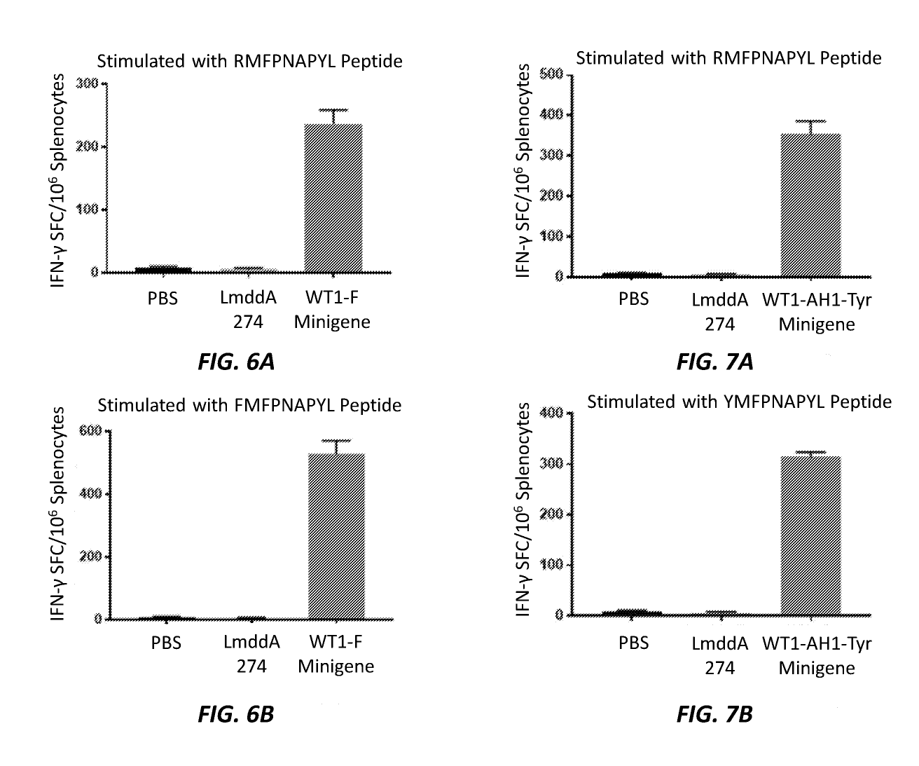

The peptide minigene expression system was used to assess nine unique heteroclitic minigenes targeting the Wilms tumor protein. This expression system is designed to facilitate cloning of panels of recombinant proteins containing distinct peptide moieties at the carboxy-terminus. This is accomplished by a simple PCR reaction utilizing a sequence encoding one of the Signal Sequence (SS)-Ubiquitin (Ub)-Antigenic Peptide constructs as a template. By using a primer that extends into the carboxy-terminal region of the Ub sequence and introducing codons for the desired peptide sequence at the 3′ end of the primer, a new SS-Ub-Peptide sequence can be generated in a single PCR reaction. The 5′ primer encoding the bacterial promoter and first few nucleotides of the signal sequence (e.g., LLO or ActA₁₋₁₀₀ secretion signal) can be the same for all constructs. The constructs generated using this strategy are represented schematically in FIGS. 3A and 3B.

One of the advantages of the minigene system is that it will be possible to load cells with multiple peptides using a single Listeria vector construct. Multiple peptides can be introduce into recombinant attenuated Listeria (e.g., Lmdda) using a modification of the single peptide expression system described above. A chimeric protein encoding multiple distinct peptides from sequential SS-Ub-Peptide sequences can be encoded in one insert. See, e.g., FIG. 3B. Shine-Dalgarno ribosome binding sites can be introduced before each SS-Ub-Peptide coding sequence to enable separate translation of each of the peptide constructs. FIG. 3B demonstrates a schematic representation of a construct designed to express three separate peptide antigens from one strain of recombinant Listeria.

To assess the expression of tLLO-WT1-heteroclitic fusion proteins by ADXS Lmdda Listeria constructs, 10 unique heteroclitic minigenes targeting the Wilms Tumor 1 protein were generated in the pAdv134 plasmid and transformed into Lmdda. The pAdv134 tLLO plasmid encodes the N-terminal LLO fragment set forth in SEQ ID NO: 59. The tLLO-WT1 heteroclitic fusion proteins comprise from N-terminal end to C-terminal end: the N-terminal LLO fragment set forth in SEQ ID NO: 59, followed by the FLAG tag set forth in SEQ ID NO: 99, followed by the ubiquitin sequence set forth in SEQ ID NO: 100, followed by a heteroclitic WT1 9-mer listed in Table 2, below.

TABLE 2
Heteroclitic WT1 Peptides.

| | WT1 9-Mer | |
|---|---|---|
| | (Heteroclitic AA | SEQ |
| Construct | Bolded and | ID |
| # | Underlined) | NO |
| 1 | FMFPNAPYL | 114 |
| 2 | YLGEQQYSV | 116 |
| 3 | YLLPAVPSL | 117 |
| 4 | YLNALLPAV | 119 |
| 5 | ALLLRTPYV | 122 |
| 6 | YLGATLKGV | 118 |
| 7 | KLYFKLSHL | 121 |
| 8 | YMTWNQMNL | 123 |
| 9 | GLRRGIQDV | 120 |
| 10 | YMFPNAPYL | 115 |

The combined WT1-tLLO-FLAG-Ub-heteroclitic phenylalanine construct (construct #1) is set forth in SEQ ID NO: 156. One additional construct (Lmdda-WT1-tLLO-P1-P2-P3-FLAG-UB-heteroclitic tyrosine minigene construct) was generated that targets 3 WT1 peptides (P1-P2-P3; SEQ ID NOS: 154 (RSDELVRHHNMHQRNMTKL), 155 (PGCNKRYFKLSHLQMHSRKHTG), and 137 (SGQAYMFPNAPYLPSCLES), respectively). Each ‘P’ peptide is comprised of 19-22 amino acids, sufficient in length to provide additional CD4 T helper epitopes. The three peptides are separated by linkers. The P3 peptide contains a heteroclitic mutation converting SGQARMFPNAPYLPSCLES (SEQ ID NO: 152) to SGQAYMFPNAPYLPSCLES (SEQ ID NO: 137). In addition to the heteroclitic P3 peptide, the Lmdda-WT1-tLLO-P1-P2-P3-FLAG-UB-heteroclitic tyrosine minigene construct contains a ubiquitin-YMFPNAPYL (SEQ ID NO: 115) moiety at the C-terminus. The combined WT1-tLLO-P1-P2-P3-FLAG-UB-heteroclitic tyrosine minigene construct is set forth in SEQ ID NO: 153. Each individual Lmdda construct was assayed by Western blot for tLLO-fusion protein expression of the unique heteroclitic WT1 minigene product.

Construct #1 (Lmdda-WT1-tLLO-FLAG-Ub-heteroclitic phenylalanine minigene construct) and the Lmdda-WT1-tLLO-P1-P2-P3-FLAG-UB-heteroclitic tyrosine minigene construct were assayed by Western blot for tLLO-fusion protein expression of the unique heteroclitic WT1 minigene product. Single colonies from plates containing Lm WT1 minigene constructs were used to inoculate an overnight culture in 6 mL of Brain Heart Infusion (BHI) broth in a dry shaking incubator at 37° C. The following day, 1:10 dilution of the original overnight culture were resuspended in 9 mL of fresh BHI and grown in the dry shaking incubator at 37° C. until reaching an OD₆₀₀=0.6. Cells were pelleted by 2-minute centrifugation at 13000 RPM. Sample supernatant were collected and run on SDS-PAGE. Samples were prepared by diluting 75 μL of sample with 25 μL of 4×LDS Sample Buffer (Cat#161-0747), boiled at 98° C. for 10 minutes, placed on ice, and then centrifuged at max speed for 10 minutes at 4° C. 13 μL of the sample was run on 4-15% precast protein gel (BioRad Cat#4561086). Protein gels were transferred using the Trans-Blot Turbo transfer apparatus (Cat#170-4155) and PVDF Midi transfer packs (Bio-Rad #170-4157). Blots were incubated with anti-FLAG monoclonal Antibody (Sigma F1804) or anti-LLO (Abcam ab200538) as primary and goat anti-mouse IgG-HRP conjugated (sc2005) as a secondary antibody. The blots were then incubated on iBind Flex (Invitrogen cat#1772866), washed, and then developed by Super Signal West Dura Extended Duration Substrate (ThermoFisher #34076); the images were developed on the Amersham Imager 600 (GE).

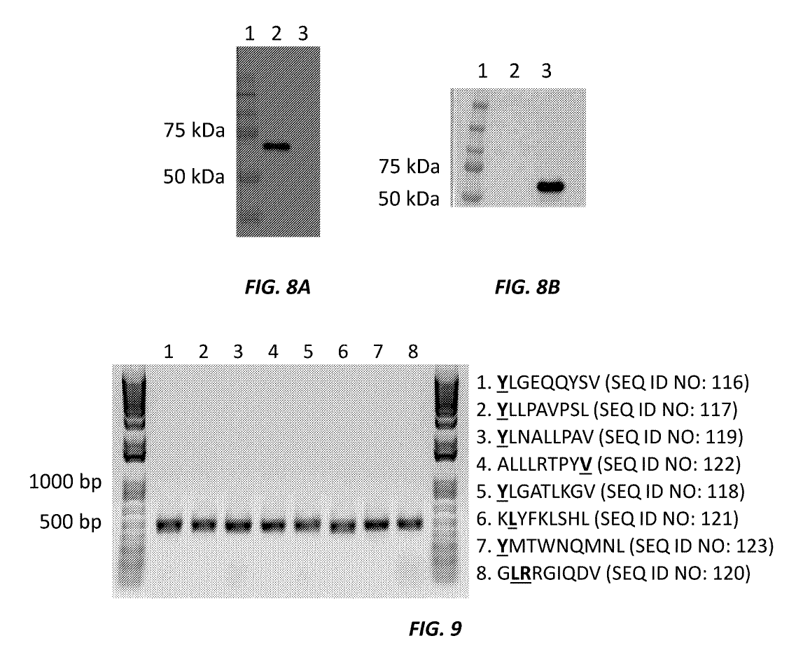

Expression and secretion of the unique tLLO-WT1-heteroclitic minigene fusion proteins was confirmed. Anti-Flag tag antibody Western blots of culture supernatant from construct #1 and the Lmdda-WT1-P1-P2-P3-YMFPNAPYL Heteroclitic tyrosine+minigene construct are shown in FIGS. 8A and 8B, respectively. We were able to detect a protein band corresponding to the correct size and identity for each individual tLLO-WT1-heteroclitic minigene fusion protein. These data demonstrate the ability for heteroclitic peptides targeting multiple peptide fragments within the WT1 protein to be generated using the pAdv134 plasmid and Lmdda Listeria strain.

For constructs #2-9 in Table 2, each individual Lmdda construct was assayed by colony PCR in order to detect plasmid DNA from each unique tLLO-fusion protein containing heteroclitic WT1 minigenes.

### Materials

| | | Catalog #/ |
|---|---|---|
| Material | Vendor | Sequence |
| DreamTaq DNA | ThermoFisher | EP0702 |
| Polymerase | | |
| Forward Primer | ThermoFisher | 5′-catcgatcactct |
| (Adv16 f)* | | gga-3′ |
| | | (SEQ ID NO: 167) |
| Reverse Primer | ThermoFisher | 5′-ctaactccaatgt |
| (Adv295 r)* | | tacttg-3′ |
| | | (SEQ ID NO: 168) |
| 10 mM dNTPs | NEB | N04475 |
| TrackIt 1 kB | ThermoFisher | 10488085 |
| Plus DNA Ladder | | |

### Procedure

The general colony PCR procedure that was used is as follows. Obtain plate with large colonies (generally, plates grown at 37° C. for 24 hours work well for this procedure). Create master mix for PCR as follows.

| Reagent | Volume (μL) |
|---|---|
| PCR water | 16 |
| DreamTaq 10× Buffer | 2 |
| Forward primer | 0.5 |
| Reverse primer | 0.5 |
| 10 mM dNTPs | 0.5 |
| Dream Taq Polymerase | 0.5 |
| | =20.0 |

Aliquot 20 μL of master mix into each PCR tube. Using a pipette tip (10-20 μL volume works best), scoop up a generous volume from one colony. Tap the pipette tip into the PCR tube several times and swirl around to dislodge the bacteria. Run the PCR reaction(s) in a thermocycler using the following PCR program.

| Step | Temp (° C.) | Time |
|---|---|---|
| 1 | 94 | 2 minutes |
| 2 | 94 | 30 seconds |
| 3 | 55* | 30 seconds |
| 4 | 72 | 1 minute |

repeat steps 2-4 an additional 29x

| | | |
|---|---|---|
| 5 | 72 | 5 minutes |
| 6 | 4 | ∞ |

Remove PCR tubes from the thermocycler, add 4 μL of 6× loading dye. Run 10 μL of each PCR reaction on a 1% agarose gel, alongside 10 μL of the 1 kb+ DNA ladder. The primers add an additional 163 base pairs to the product. The forward primer binds 70 base pairs upstream of the 3′ end of tLLO (includes the XhoI site). The reverse primer binds 93 base pairs downstream of the stop sites (includes the XmaI site).

Representative colony PCR results showing Lmdda strains containing pAdv134 WT1-heteroclitic plasmids #2-9 from Table 2 are shown in FIG. 9. We were able to detect a DNA band corresponding to the correct size and identity for each individual tLLO-WT1-heteroclitic minigene plasmid. These data demonstrate the ability for heteroclitic peptides targeting multiple peptide fragments within the WT1 protein to be generated using the pAdv134 plasmid and Lmdda Listeria strain, which indicates that such constructs can be used as therapeutic compositions to target WT1 to create or enhance immune responses against WT1 and WT1-expressing cancers and tumors.

### Example 5. Evaluation of Immunogenicity of WT1 Constructs

To assess the generation of WT1-specific T cell responses in AAD mice using two different WT1 constructs, ELISpots was performed to determine the desired vaccine-induced Ag-specific responses. The AAD mice (B6.Cg-Tg(HLA-A/H2-D)2Enge/J; The Jackson Laboratory—Stock No.: 004191) are transgenic mice that express an interspecies hybrid class I MHC gene, AAD, which contains the alpha-1 and alpha-2 domains of the human HLA-A2.1 gene and the alpha-3 transmembrane and cytoplasmic domains of the mouse H-2D^{d} gene, under the direction of the human HLA-A2.1 promoter. This transgenic strain enables the modeling of human T cell immune responses to HLA-A2 presented antigens, and may be useful in testing of vaccines for infectious diseases or cancer therapy. The immunization schedule is provided in Table 3. The mice that were used were female C57BL/6 mice aged 8-10 weeks.

TABLE 3
Immunization Schedule.

| | | | Dose 1 | Dose 2 | |
|---|---|---|---|---|---|
| Vaccine/ | Titer- | Mice/ | (IP/200 μL/ | (IP/200 μL/ | |
| Group | CFU/mL | Group | mouse) | mouse) | Harvest |
| 1 - PBS | N/A | 5 | Day 0 | Day 12 | Day 18 |
| 2 - LmddA 274 | ~1 × 10⁹ | 5 | Day 0 | Day 12 | Day 18 |
| 3 - WT1Fm-FLAG-Ub-9 | ~1 × 10⁹ | 5 | Day 0 | Day 12 | Day 18 |
| (WT1-F minigene) | | | | | |
| 4 - LmddA + pAdv134- | ~1 × 10⁹ | 5 | Day 0 | Day 12 | Day 18 |
| WT1m:Ub-9 | | | | | |
| (WT1-AH1-Tyr minigene) | | | | | |

Vaccine Preparations.

Briefly, each glycerol stock was streaked over required nutrient plate and grown overnight. A single colony was used for growth in an overnight culture of Brain Heart Infusion (BHI) broth under antibiotic selection. Overnight cultures were used at a 1:10 (vol/vol) dilution to inoculate fresh BHI broth. Bacteria were incubated in an orbital shaker for 1-3 hours at 37° C. to mid-log phase, an OD of ˜0.6-0.7. Mice were infected with 1×10⁹ CFU Lm by i.p. inoculation in PBS.

ELISPOT.

On day 18, mice were sacrificed by CO₂ asphyxiation in accordance with IACUC protocols, spleens were harvested, and splenocyte single-cell suspensions were plated on 96-well plates and stimulated with either the wild-type or heteroclitic peptide (Table 4). Similar experiments are done with other wild-type and heteroclitic peptide pairs (Table 5). An ELISPOT assay was used to enumerate antigen specific CD8 T Cells responding to either the wild-type or heteroclitic peptides. The full ELISPOT protocol was as per CTL immunospot (www.immunospot.com/resources/protocols/ELISPOT-protocol.htm).

TABLE 4
Wild-Type and Heteroclitic WT1 Peptides.

| Wild-Type | Negative | Heteroclitic |
|---|---|---|
| Peptide | Control | Peptides |
| RMFPNAPYL | RPMI | FMFPNAPYL |
| (SEQ ID NO: 101) | Empty | (SEQ ID NO: 114) |
| | Media | YMFPNAPYL |
| | | (SEQ ID NO: 115) |

TABLE 5
Wild-Type and Heteroclitic WT1 Peptides.

| Wild-Type | Heteroclitic |
|---|---|
| SLGEQQYSV | YLGEQQYSV |
| (SEQ ID NO: 102) | (SEQ ID NO: 116) |
| ALLPAVPSL | YLLPAVPSL |
| (SEQ ID NO: 103) | (SEQ ID NO: 117) |
| DLNALLPAV | YLNALLPAV |
| (SEQ ID NO: 105) | (SEQ ID NO: 119) |
| ALLLRTPYS | ALLLRTPYV |
| (SEQ ID NO: 108) | (SEQ ID NO: 122) |
| NLGATLKGV | YLGATLKGV |
| (SEQ ID NO: 104) | (SEQ ID NO: 118) |
| KRYFKLSHL | KLYFKLSHL |
| (SEQ ID NO: 107) | (SEQ ID NO: 121) |
| CMTWNQMNL | YMTWNQMNL |
| (SEQ ID NO: 109) | (SEQ ID NO: 123) |
| GVFRGIQDV | GLRRGIQDV |
| (SEQ ID NO: 106) | (SEQ ID NO: 120) |

A generic ELISPOT protocol is provided below.

DAY 0 (Sterile Conditions).

Prepare Capture Solution by diluting the Capture Antibody according to your specific protocol. Many cytokines benefit from pre-wetting the PVDF membrane with 70% ethanol for 30 sec and washing with 150 μL of PBS three times before adding 80 μL of the Capture Solution into each well. Incubate plate overnight at 4° C. in a humidified chamber.

DAY 1 (Sterile Conditions).

Prepare CTL-Test™ Medium by adding 1% fresh L-glutamine. Prepare antigen/mitogen solutions at 2× final concentration in CTL-Test™ Medium. Decant plate with coating antibody from Day 0 and wash one time with 150 μL PBS. Plate antigen/mitogen solutions, 100 μL/well. After thawing PBMC or isolating white blood cells with density gradient, adjust PBMC to desired concentration in CTL-Test™ Medium, e.g., 3 million/mL corresponding to 300,000 cells/well (however, cell numbers can be adjusted according to expected spot counts since 100,000-800,000 cells/well will provide linear results). While processing PBMC and until plating, keep cells at 37° C. in humidified incubator, 5-9% CO₂. Plate PBMC, 100 μL/well using large orifice tips. Once completed, gently tap the sides of the plate and immediately place into a 37° C. humidified incubator, 5-9% CO₂. Incubate for 24-72 hours depending on your cytokine. Do not stack plates. Avoid shaking plates by carefully opening and shutting incubator door. Do not touch plates during incubation.

DAY 2.

Prepare Wash Solutions for the day: PBS, distilled water and Tween-PBS. Prepare Detection Solution by diluting Detection Antibody according to your specific protocol. Wash plate two times with PBS and then two times with 0.05% Tween-PBS, 200 μL/well each time. Add 80 μL/well Detection Solution. Incubate at RT, 2 h. Prepare Tertiary Solution by diluting the Tertiary Antibody according to your specific protocol. Wash plate three times with 0.05% Tween-PBS, 200 μL/well. Add 80 μL/well of Strep-AP Solution. Incubate at RT, 30 min. Prepare Developer Solution according to your specific protocol. Wash plate two times with 0.05% Tween-PBS, and then two times with distilled water, 200 μL/well each time. Add Developer Solution, 80 μL/well. Incubate at RT, 10-20 min. Stop reaction by gently rinsing membrane with tap water, decant, and repeat three times. Remove protective underdrain of the plate and rinse back of plate with tap water. Air dry plate for 2 hours face-down in running hood or on paper towels for 24 hours on bench top. Scan and count plate.

HLA-A2 transgenic B6 mice were vaccinated as described, and splenocytes were stimulated ex vivo with specific WT1 peptides (RMFPNAPYL (SEQ ID NO: 101), FMFPNAPYL (SEQ ID NO 114)) and analyzed by IFNg ELISpot assay. Heteroclitic vaccination (WT1-F minigene: FMFPNAPYL; SEQ ID NO: 114) induces Ag-specific T cell responses in immunized HLA2 transgenic mice. In addition, heteroclitic vaccination elicits T cell responses that cross-react with the native WT1 tumor antigen (RMFPNAPYL; SEQ ID NO: 101). The data demonstrate that vaccination with the WT1-F heteroclitic minigene vaccine can elicit T cells that are cross-reactive with the WT1-native tumor antigen (RMFPNAPYL; SEQ ID NO: 101). Overall, the data demonstrate that the heteroclitic minigene vaccine can elicit T cells that cross-react with the native tumor antigen.

HLA-A2 transgenic B6 mice were vaccinated as described and splenocytes were harvested. The ability of T cells to produce IFNg in response to vaccine-specific YMFPNAPYL peptide (SEQ ID NO: 115) or native WT1 peptide (RMFPNAPYL; SEQ ID NO: 101) was determined by IFNg ELISpot assay. Heteroclitic vaccination (WT1-AH1-Tyr minigene: YMFPNAPYL; SEQ ID NO: 115) induces Ag-specific T cell responses in immunized HLA2 transgenic mice. In addition, heteroclitic vaccination elicits T cell responses that cross-react with the native WT1 tumor antigen (RMFPAPYL; SEQ ID NO: 101).

We claim:

1. A minigene construct comprising a nucleic acid comprising an open reading frame encoding a chimeric polypeptide, wherein the chimeric polypeptide comprises:
(a) a bacterial secretion signal sequence;
(b) a ubiquitin protein; and
(c) an antigenic Wilms tumor protein (WT1) peptide,
wherein the bacterial secretion signal sequence, the ubiquitin, and the antigenic WT1 peptide are arranged in tandem from the amino-terminal end to the carboxy-terminal end of the chimeric polypeptide.

2. The minigene construct of claim 1, wherein the antigenic WT1 peptide comprises a heteroclitic mutant WT1 peptide.

3. The minigene construct of claim 2, wherein the heteroclitic mutant WT1 peptide comprises the sequence set forth in any one of SEQ ID NOS: 114-137, 159, 160, 162, and 163.

4. The minigene construct of claim 3, wherein the heteroclitic mutant WT1 peptide comprises the sequence set forth in SEQ ID NO: 114, 115, 162, or 163.

5. The minigene construct of claim 4, wherein the chimeric polypeptide comprises the sequence set forth in SEQ ID NO: 156, 157, 165, or 166.

6. The minigene construct of any claim 1, wherein the antigenic WT1 peptide comprises a native WT1 peptide.

7. The minigene construct of claim 6, wherein the native WT1 peptide comprises the sequence set forth in SEQ ID NO: 95 or 158.

8. The minigene construct of claim 7, wherein the chimeric polypeptide comprises the sequence set forth in SEQ ID NO: 164.

9. The minigene construct of any one of claims 1-4, 6, and 7, wherein the chimeric polypeptide further comprises one or more additional antigenic WT1 peptides between the bacterial signal sequence and the ubiquitin protein.

10. The minigene construct of claim 9, wherein the one or more additional antigenic WT1 peptides comprise two or more additional antigenic WT1 peptides.

11. The minigene construct of claim 10, wherein the two or more additional antigenic WT1 peptides are fused directly to each other without intervening sequence.

12. The minigene construct of claim 10, wherein the two or more additional antigenic WT1 peptides are linked to each other via peptide linkers.

13. The minigene construct of any one of claims 9-12, wherein the one or more additional antigenic peptides comprise a first additional antigenic WT1 peptide comprising the sequence set forth in SEQ ID NO: 154, a second additional antigenic WT1 peptide comprising the sequence set forth in SEQ ID NO: 155, and a third additional antigenic WT1 peptide comprising the sequence set forth in SEQ ID NO: 136.

14. The minigene construct of any one of claims 9-12, wherein the one or more additional antigenic peptides comprise a first additional antigenic WT1 peptide comprising the sequence set forth in SEQ ID NO: 154, a second additional antigenic WT1 peptide comprising the sequence set forth in SEQ ID NO: 155, and a third additional antigenic WT1 peptide comprising the sequence set forth in SEQ ID NO: 137.

15. The minigene construct of claim 14, wherein the chimeric polypeptide comprises the sequence set forth in SEQ ID NO: 153.

16. The minigene construct of any preceding claim, wherein the bacterial secretion signal sequence is a listeriolysin O (LLO) secretion signal sequence.

17. The minigene construct of claim 16, wherein the LLO secretion signal sequence comprises the sequence set forth in SEQ ID NO: 59 or SEQ ID NO: 150.

18. The minigene construct of any preceding claim, wherein the ubiquitin protein comprises the sequence set forth in SEQ ID NO: 100.

19. The minigene construct of any preceding claim, wherein the antigenic WT1 peptide comprises the sequence set forth in any one of SEQ ID NOS: 95, 114-137, 158, 162, and 163, the bacterial secretion signal sequences comprises the sequence set forth in SEQ ID NO: 59, and the ubiquitin protein comprises the sequence set forth in SEQ ID NO: 100.

20. The minigene construct of any preceding claim, wherein the minigene construct comprises two or more open reading frames, each encoding a chimeric polypeptide comprising:
(a) a bacterial secretion signal sequence;
(b) a ubiquitin protein; and
(c) an antigenic WT1 peptide,
wherein the bacterial secretion signal sequence, the ubiquitin, and the antigenic WT1 peptide are arranged in tandem from the amino-terminal end to the carboxy-terminal end of each chimeric polypeptide, and
wherein the antigenic WT1 peptide is different in each chimeric polypeptide, and
wherein the minigene construct comprises a Shine-Dalgarno ribosome binding site nucleic acid sequence between each pair of open reading frames.

21. A chimeric polypeptide encoded by the minigene construct of any preceding claim.

22. A recombinant Listeria strain comprising the minigene construct of any one of claims 1-20.

23. A recombinant Listeria strain comprising a nucleic acid comprising a first open reading frame encoding a fusion polypeptide, wherein the fusion polypeptide comprises a PEST-containing peptide fused to an antigenic peptide selected from a native Wilms tumor protein (WT1), an immunogenic fragment of the native WT1, and a WT1 peptide comprising the sequence set forth in SEQ ID NO: 114 or SEQ ID NO: 136.

24. The recombinant Listeria strain of claim 23, wherein the fusion polypeptide comprises the native WT1.

25. The recombinant Listeria strain of claim 23, wherein the fusion polypeptide comprises the immunogenic fragment of the native WT1.

26. The recombinant Listeria strain of claim 25, wherein the immunogenic fragment of the native WT1 comprises the sequence set forth in SEQ ID NO: 95 or SEQ ID NO: 139.

27. The recombinant Listeria strain of claim 23, wherein the fusion polypeptide comprises the WT1 peptide comprising the sequence set forth in SEQ ID NO: 114 or SEQ ID NO: 136.

28. The recombinant Listeria strain of any one of claims 23-27, wherein the fusion polypeptide comprises the PEST-containing peptide fused to two or more antigenic WT1 peptides, wherein at least one of the two or more antigenic WT1 peptides is selected from the native Wilms tumor protein (WT1), the immunogenic fragment of the native WT1, and the WT1 peptide comprising the sequence set forth in SEQ ID NO: 114 or SEQ ID NO: 136, and wherein the two or more antigenic WT1 peptides are the same or different.

29. The recombinant Listeria strain of any one of claims 23-28, wherein the two or more antigenic WT1 peptides are fused directly to each other without intervening sequence.

30. The recombinant Listeria strain of any one of claims 23-28, wherein the two or more antigenic WT1 peptides are linked to each other via peptide linkers.

31. The recombinant Listeria strain of any one of claims 23-30, wherein the fusion polypeptide further comprises one or more peptide tags N-terminal and/or C-terminal to the one or more of the native Wilms tumor protein (WT1), the immunogenic fragment of the native WT1, and the WT1 peptide comprising the sequence set forth in SEQ ID NO: 114 or SEQ ID NO: 136.

32. The recombinant Listeria strain of claim 31, wherein the one or more peptide tags comprise one or more of the following: 3×FLAG tag; 6×His tag; SIINFEKL tag, and the FLAG tag set forth in SEQ ID NO: 99.

33. The recombinant Listeria strain of any one of claims 23-32, wherein the PEST-containing peptide is on the N-terminal end of the fusion polypeptide.

34. The recombinant Listeria strain of any one of claims 23-33, wherein the PEST-containing peptide is an N-terminal fragment of LLO.

35. The recombinant Listeria strain of claim 34, wherein the N-terminal fragment of LLO comprises the sequence set forth in SEQ ID NO: 59.

36. The recombinant Listeria strain of any one of claims 22-35, wherein the nucleic acid is operably integrated into the Listeria genome.

37. The recombinant Listeria strain of any one of claims 22-35, wherein the nucleic acid is in an episomal plasmid.

38. The recombinant Listeria strain of any one of claims 22-37, wherein the nucleic acid does not confer antibiotic resistance upon the recombinant Listeria strain.

39. The recombinant Listeria strain of any one of claims 22-38, wherein the recombinant Listeria strain is an attenuated, auxotrophic Listeria strain.

40. The recombinant Listeria strain of claim 39, wherein the attenuated Listeria strain comprises a mutation in one or more endogenous genes that inactivates the one or more endogenous genes.

41. The recombinant Listeria strain of claim 40, wherein the one or more endogenous genes comprise prfA.

42. The recombinant Listeria strain of claim 40, wherein the one or more endogenous genes comprise actA.

43. The recombinant Listeria strain of claim 40, wherein the one or more endogenous genes comprise actA and inlB.

44. The recombinant Listeria strain of claim 40, wherein the one or more endogenous genes comprise actA, dal, and dat.

45. The recombinant Listeria strain of any one of claims 22-44, wherein the nucleic acid comprises a second open reading frame encoding a metabolic enzyme.

46. The recombinant Listeria strain of claim 45, wherein the metabolic enzyme is an alanine racemase enzyme or a D-amino acid aminotransferase enzyme.

47. The recombinant Listeria strain of any one of claims 22-46, wherein the fusion polypeptide is expressed from an hly promoter.

48. The recombinant Listeria strain of any one of claims 22-47, wherein the recombinant Listeria strain is a recombinant Listeria monocytogenes strain.

49. The recombinant Listeria strain of any one of claims 22-33, wherein the recombinant Listeria strain is an attenuated Listeria monocytogenes strain comprising a deletion of or inactivating mutation in prfA, wherein the nucleic acid is in an episomal plasmid and comprises a second open reading frame encoding a D133V PrfA mutant protein.

50. The recombinant Listeria strain of any one of claims 22-33, wherein the recombinant Listeria strain is an attenuated Listeria monocytogenes strain comprising a deletion of or inactivating mutation in actA, dal, and dat, wherein the nucleic acid is in an episomal plasmid and comprises a second open reading frame encoding an alanine racemase enzyme or a D-amino acid aminotransferase enzyme, and wherein the PEST-containing peptide is an N-terminal fragment of LLO.

51. The recombinant Listeria strain of any one of claims 22-33, wherein recombinant Listeria strain is an attenuated Listeria monocytogenes strain comprising a deletion of or inactivating mutation in actA and inlB, wherein the nucleic acid is genomically integrated, and wherein the PEST-containing peptide is an ActA protein or a fragment thereof.

52. An immunogenic composition comprising the recombinant Listeria strain of any one of claims 22-51 and an adjuvant.

53. The immunogenic composition of claim 52, wherein the adjuvant comprises a detoxified listeriolysin O (dtLLO), a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

54. A method of inducing an immune response against a WT1-expressing tumor or cancer in a subject, comprising administering to the subject the recombinant Listeria strain of any one of claims 22-51 or the immunogenic composition of any one of claims 52-53.

55. A method of preventing or treating a WT1-expressing tumor or cancer in a subject, comprising administering to the subject the recombinant Listeria strain of any one of claims 22-51 or the immunogenic composition of any one of claims 52-53.
